SEQUENCE LISTING (49 sequences)

SEQ ID NO: 1 - protein, 7 aa, Human immunodeficiency virus 1
KRRQRRR

SEQ ID NO: 2 - protein, 16 aa, Unknown
  Description of Unknown Penetratin PTD peptide
RQIKIWFQNRRMKWKK

SEQ ID NO: 3 - protein, 34 aa, Human herpesvirus 1
DAATATRGRSAASRPTERPRAPARSASRPRRPVD

SEQ ID NO: 4 - protein, 16 aa, Unknown
  Description of Unknown Kaposi FGF signal peptide
AAVALLPAVLLALLAP

SEQ ID NO: 5 - protein, 16 aa, Unknown
  Description of Unknown Kaposi FGF signal peptide
AAVLLPVLLPVLLAAP

SEQ ID NO: 6 - protein, 15 aa, Homo sapiens
VTVLALGALAGVGVG

SEQ ID NO: 7 - protein, 17 aa, Human immunodeficiency virus 1
GALFLGWLGAAGSTMGA

SEQ ID NO: 8 - protein, 17 aa, Caiman crocodylus
MGLGLHLLVLAAALQGA

SEQ ID NO: 9 - protein, 24 aa, Homo sapiens
LGTYTQDFNKFHTFPQTAIGVGAP

SEQ ID NO: 10 - protein, 26 aa, Artificial Sequence
  Description of Artificial Sequence Synthetic peptide
GWTLNSAGYLLKINLKALAALAKKIL

SEQ ID NO: 11 - protein, 16 aa, Artificial Sequence
  Description of Artificial Sequence Synthetic peptide
TPPKKKRKVEDPKKKK

SEQ ID NO: 12 - protein, 7 aa, Artificial Sequence
  Description of Artificial Sequence Synthetic peptide
RRRRRRR

SEQ ID NO: 13 - protein, 18 aa, Artificial Sequence
  Description of Artificial Sequence Synthetic peptide
KLALKLALKALKAALKLA

SEQ ID NO: 14 - protein, 31 aa, Artificial Sequence
  Description of Artificial Sequence Synthetic polypeptide
WWETWKPFQCRICMRNFSTRQARRNHRRRHR

SEQ ID NO: 15 - protein, 16 aa, Artificial Sequence
  Description of Artificial Sequence Synthetic peptide
GKINLKALAALAKKIL

SEQ ID NO: 16 - protein, 16 aa, Artificial Sequence
  Description of Artificial Sequence Synthetic peptide
RVIRVWFQNKRCKDKK

SEQ ID NO: 17 - protein, 39 aa, Artificial Sequence
  Description of Artificial Sequence Synthetic polypeptide
GRKKRRQRRRPPQGRKKRRQRRRPPQGRKKRRQRRRPPQ

SEQ ID NO: 18 - protein, 22 aa, Artificial Sequence
  Description of Artificial Sequence Synthetic peptide
GEQIAQLIAGYIDIILKKKKSK

SEQ ID NO: 19 - DNA, 20 bp, Artificial Sequence
  Description of Artificial Sequence Synthetic primer
tgcgagaggtcagcacccgc

SEQ ID NO: 20 - DNA, 23 bp, Artificial Sequence
  Description of Artificial Sequence Synthetic primer
caggtcaaacttccggaagatgg

SEQ ID NO: 21 - DNA, 20 bp, Artificial Sequence
  Description of Artificial Sequence Synthetic primer
atctgccaaggagccctagg

SEQ ID NO: 22 - DNA, 20 bp, Artificial Sequence
  Description of Artificial Sequence Synthetic primer
tagtgttccagcttgggcag

SEQ ID NO: 23 - DNA, 28 bp, Artificial Sequence
  Description of Artificial Sequence Synthetic primer
aaagatggaaaaccactattgccagagc

SEQ ID NO: 24 - DNA, 27 bp, Artificial Sequence
  Description of Artificial Sequence Synthetic primer
gacctcttctaatctataatcaggagg

SEQ ID NO: 25 - DNA, 21 bp, Artificial Sequence
  Description of Artificial Sequence Synthetic primer
tcctccctggagaagagctac

SEQ ID NO: 26 - DNA, 21 bp, Artificial Sequence
  Description of Artificial Sequence Synthetic primer
taaagccatgccaatctcatc

SEQ ID NO: 27 - RNA, 21 nt, Artificial Sequence
  Description of Artificial Sequence Synthetic oligonucleotide
ggaacaacguggacccauauu

SEQ ID NO: 28 - RNA, 21 nt, Artificial Sequence
  Description of Artificial Sequence Synthetic oligonucleotide
uauggguccacguuguuccuu

SEQ ID NO: 29 - RNA, 21 nt, Artificial Sequence
  Description of Artificial Sequence Synthetic oligonucleotide
gugaaggaguugcggacaauu

SEQ ID NO: 30 - RNA, 21 nt, Artificial Sequence
  Description of Artificial Sequence Synthetic oligonucleotide
uuguccgcaacuccuucacuu

SEQ ID NO: 31 - RNA, 21 nt, Artificial Sequence
  Description of Artificial Sequence Synthetic oligonucleotide
uagagaccauguuccgauuuu

SEQ ID NO: 32 - RNA, 21 nt, Artificial Sequence
  Description of Artificial Sequence Synthetic oligonucleotide
aaucggaacauggucucuauu

SEQ ID NO: 33 - RNA, 21 nt, Artificial Sequence
  Description of Artificial Sequence Synthetic oligonucleotide
gaagggccaugccuacucuuu

SEQ ID NO: 34 - RNA, 21 nt, Artificial Sequence
  Description of Artificial Sequence Synthetic oligonucleotide
agaguaggcauggcccuucuu

SEQ ID NO: 35 - RNA, 25 nt, Artificial Sequence
  Description of Artificial Sequence Synthetic oligonucleotide
cuaccaagcuguuccggaaugauuu

SEQ ID NO: 36 - RNA, 21 nt, Artificial Sequence
  Description of Artificial Sequence Synthetic oligonucleotide
aucaucgacagcuugguaguu

SEQ ID NO: 37 - RNA, 21 nt, Artificial Sequence
  Description of Artificial Sequence Synthetic oligonucleotide
ccgaggagguugaaaguaauu

SEQ ID NO: 38 - RNA, 21 nt, Artificial Sequence
  Description of Artificial Sequence Synthetic oligonucleotide
uuacuuucaaccuccucgguu

SEQ ID NO: 39 - RNA, 28 nt, Artificial Sequence
  Description of Artificial Sequence Synthetic oligonucleotide
ggaacuacccgaaaccaaccaauuuuuu

SEQ ID NO: 40 - RNA, 21 nt, Artificial Sequence
  Description of Artificial Sequence Synthetic oligonucleotide
aauguguucggguaguuccuu

SEQ ID NO: 41 - RNA, 28 nt, Artificial Sequence
  Description of Artificial Sequence Synthetic oligonucleotide
uggaaacgcuaauuuccaaaggaauuuu

SEQ ID NO: 42 - RNA, 25 nt, Artificial Sequence
  Description of Artificial Sequence Synthetic oligonucleotide
aucuugaauagcguuuuuccccauu

SEQ ID NO: 43 - protein, 27 aa, Artificial Sequence
  Description of Artificial Sequence Synthetic peptide
DPMSSTYIEELGKREVTIPPKYRELLA

SEQ ID NO: 44 - protein, 66 aa, Unknown
  Description of Unknown Calpain inhibitor polypeptide
MSSRPLESPPPYRPDEFKPNHYAPSNDIYGGEMHVRPMLSQPAYSFYPEDEILHFYKWTSPPGVIR

SEQ ID NO: 45 - protein, 149 aa, Homo sapiens
RAVVKEPLLPPEDDTRDNVYYYDEEGGGEEDQDFDLSQLHRGLDARPEVTRNDVAPTLMSVPRYLPRPANPDEIGNFIDENLKAADTDPTAPPYDSLLVFDYEGSGSEAASLSSLNSSESDKDQDYDYLNEWGNRFKKLADMYGGGEDD

SEQ ID NO: 46 - protein, 7 aa, Homo sapiens
  MOD_RES (5)..(5): Any amino acid
TIPPXYR

SEQ ID NO: 47 - protein, 6 aa, Homo sapiens
EYPPIT

SEQ ID NO: 48 - protein, 6 aa, Pseudomonas aeruginosa
CSKKKK

SEQ ID NO: 49 - protein, 4 aa, Artificial Sequence
  Description of Artificial Sequence Synthetic caspase inhibitor peptide
DEVD

(19) United States
(12) Patent Application Publication
(10) Pub. No.: US 2011/0117106 A1
(43) Pub. Date: May 19, 2011

(54) USES OF CALPAIN INHIBITORS TO INHIBIT INFLAMMATION

(76) Inventor: Alice Prince, Larchmont, NY (US)

(21) Appl. No.: 12/921,366

(22) PCT Filed: Mar. 6, 2009
(86) PCT No.: PCT/US09/36381

§ 371 Date: Nov. 24, 2010

Related U.S. Application Data
(60) Provisional application No. 61/034,265, filed on Mar. 6, 2008.

(51) Int. Cl. A61K 39/395; A61K 31/56; A61K 31/427; A61K 31/4725; A61K 31/496; A61K 31/472; A61K 31/341; A61K 31/713; A61K 38/05; A61K 31/336; A61K 31/192; A61K 31/404; A61K 38/06; A61K 38/16; A61K 38/02; A61K 31/7105; A61P 11/06; A61P 31/00; A61P 11/00; A61P 11/08; A61P 29/00

(52) U.S. Cl. 424/158.1; 514/171; 514/365; 514/307; 514/253.01; 514/471; 514/44 A; 514/20.2; 514/475; 514/570; 514/419; 514/1.7; 514/2.3

(57) ABSTRACT

Calpains target junctional components that normally seal the epithelium, forming tight junctions. This selective targeting by calpains facilitates the transmigration of leukocytes across the epithelium and into tissue spaces where they can cause inflammation. The present disclosure provides methods of using calpain inhibitors to prevent epithelial junction reorganization such that leukocyte transmigration is inhibited and accordingly, inflammation reduced or prevented. These methods can at least be used to reduce respiratory inflammation by preventing leukocyte accumulation in pulmonary airways.

This application claims the benefit of and priority to U.S. provisional patent application Ser. No. 61/034,265 filed Mar. 6, 2008, the disclosure of all of which is hereby incorporated by reference in its entirety for all purposes.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described herein.

This invention was made with government support under grant number HL73989 awarded by the National Institutes of Health. The government has certain rights in the invention.

### BACKGROUND OF THE INVENTION

Inflammation is a reaction to cellular injury that includes leukocyte infiltration, redness, pain and swelling, called the inflammatory response. The inflammatory response serves the purpose of eliminating harmful agents from the body. There are a wide range of insults that can initiate an inflammatory response, such as infection, allergens, autoimmune stimuli, immune response to transplanted tissue, toxins, ischemia/reperfusion, hypoxia, and mechanical or thermal trauma. The body's response becomes an agent of disease when inflammation results in inappropriate injury to host tissues in the process of eliminating the targeted agent, or responding to a traumatic insult.

Airway epithelial cells provide both signaling and barrier functions to protect the lungs from inhaled pathogens. Epithelial cells of the airway lumen are tightly apposed via adherens and tight junction proteins and infiltrating phagocytes must migrate between these cells reach invading pathogens. An increased presence of inflammatory cells or leukocytes can cause excessive inflammatory responses in the airway and block air exchange and respiratory failure. Respiratory tract infection is the sixth major cause of death in the US. The infection can be the result of direct damage caused by a pathogen and indirect damage caused by the inflammatory response to infection or trauma. Excessive inflammatory responses are characteristic to a number of respiratory diseases including bacterial infections, viral infections, chronic obstructive respiratory disease (COPD), cystic fibrosis and asthma.

It has been recognized in the medical art that compounds which affect inflammation have potential both as treatments for a number of disease states and as research tools. Often it is revealed that those compounds which have promising effects for controlling inflammation have unacceptable toxicity profiles, are prohibitively difficult or expensive to make, or both.

Despite the current level of knowledge and available treatments, there remains a need in the art for further medicaments or compounds that can be utilized for affecting inflammation or preventing or treating an inflammation-related condition. Thus there is a need to identify agents which inhibit leukocyte transmigration through epithelial cell layers and have the potential to treat conditions characterized by conditions of inflammation. This invention addresses these needs.

### SUMMARY OF THE INVENTION

Various leukocytes are involved in the initiation and maintenance of inflammation. These cells must be able to get to the site of injury from their usual location in the blood, therefore mechanisms exist to recruit and direct leukocytes to the appropriate place. For example, leukocytes transmigrate across the endothelium via the process of diapedesis. Chemokine gradients stimulate the adhered leukocytes to move between endothelial cells and pass the basement membrane into tissues or tissue spaces.

This disclosure demonstrates that inflammation can be reduced or prevented by blocking the migration of leukocytes, for example polymorphic nuclear cells (PMNs), through the paracellular space between epithelial cells in an endothelium. In various aspects, the invention provides methods that involve the use of calpain inhibitors, in order to prevent or inhibit the re-organization of epithelial junctions in the paracellular space such that leukocyte migration is blocked. By blocking the transmigration of leukocytes through the epithelium, calpain inhibitors can thereby be used to treat or prevent inflammatory conditions.

In one aspect, the methods described herein relate to a method for reducing inflammation in a subject, the method comprising administering at least one calpain inhibitor to the respiratory epithelial cells in the airway of the subject, wherein the at least one calpain inhibitor is administered in an amount sufficient to inhibit leukocyte transmigration across epithelial tight junctions between the respiratory epithelial cells, and wherein the at least one calpain inhibitor(s) is capable of inhibiting the protease activity of calpain 1 and/or calpain 2.

In various aspects, the invention provides a method for reducing inflammation in a subject, the method comprising administering at least one calpain inhibitor to a subject, so as to inhibit leukocyte transmigration across an epithelium. The epithelium can be, for example, an airway epithelium, a pulmonary epithelium, or a gastrointestinal epithelium. In one aspect, the epithelium comprises a tight junction. In another aspect the epithelium comprises an adherens junction.

In one aspect, administering the at least one calpain inhibitor can comprise directly contacting airway epithelial cells with the at least one calpain inhibitor. In another aspect, the at least one calpain inhibitor reduces that activity of the at least one calpain 1. In another aspect, the at least one calpain inhibitor reduces that activity of calpain 2. In still another aspect, the at least one calpain inhibitor reduces that activity of both calpain 1 and calpain 2. In another aspect, the at least one calpain inhibitors are capable of inhibiting calpain 1 and/or calpain 2 such that calpain-mediated cleavage of both occludin and E-cadherin is inhibited. In another aspect, the at least one calpain inhibitors are capable of inhibiting calpain 1 and/or calpain 2 such that calpain-mediated cleavage of occludin, E-cadherin and Ezrin is inhibited

In another aspect, administering the at least one calpain inhibitor can comprise transport or diffusion of the at least one calpain inhibitor across one of more biological membranes in a cell. In one aspect, the administering comprises delivering the at least one calpain inhibitor into the airway of the subject by inhalation. The inhalation can comprise the use of a nebulizer.

In one aspect, the inflammation of the subject occurs at a pulmonary site. In one aspect, the inflammation of the subject is in danger of occurring at a pulmonary site. In some aspects, the subject suffers from: asthma or an asthma exacerbation; chronic obstructive pulmonary disease; an opportunistic pathogenic infection of cystic fibrosis; a respiratory infection; pneumonia; a ventilator-associated pneumonia; an obstructive airway disease or condition; an eosinophil related disorder; bronchial condition; or pulmonary inflammation.

In one aspect, the at least one calpain inhibitor inhibits E-cadherin cleavage, occludin cleavage, or ezrin cleavage. In one aspect, the at least one calpain inhibitor is administered in an amount sufficient to reduce epithelial junction or tight junction reorganization. In one aspect, the at least one calpain inhibitor is administered in an amount sufficient to reduce leukocyte accumulation in the airway lumen in the subject. In one aspect, the at least one calpain inhibitor is administered in an amount sufficient to reduce leukocyte accumulation in the gut lumen in the subject.

In one aspect, the subject suffers from an inflammation-induced loss of more than about 50 percent of predicted lung function; or of about 51 to about 65 percent of predicted lung function; or of more than about 65 percent of predicted lung function.

In one aspect, the at least one calpain inhibitor comprises a small molecule, a protein, a peptide, a peptidomimetic, small interfering RNA, a short hairpin RNA, a microRNA, and an anti-calpain antibody, and derivative thereof. In one aspect, the at least one calpain inhibitor acts or binds to an active site of calpain. In one aspect, the nucleic acid sequence of the small interfering RNA is selected from the sequences shown in any of SEQ ID NOs: 27-42. In still another aspect, the nucleic acid sequence of the small interfering RNA has at least 95%, 96%, 97%, 98%, or 99% nucleic acid sequence identity the sequences shown in any of SEQ ID NOs: 27-42.

The active site directed inhibitor can comprise, for example, Calpain Inhibitor Peptide, Calpain Inhibitor I, N-acetly-L-L-norleucinal, ALLN, Calpain Inhibitor II, N-acetly-L-L-methional, ALLM, Calpain Inhibitor III, Calpain Inhibitor IV, Calpain Inhibitor V, Calpeptin. benzyloxycarbonyldipeptidyl aldehyde, trans-Epoxy succinyl-L-leucylamido-(4-guanidino)butane, and Z-Leu-Leu-CHO, E64D, SJA6017, N-(4-fluorophenylsulfonyl)-L-valyl-L-leucinal, AK295, benzyloxycarbony-Leu-aminobutyric acid-CONH(CH2)3-morpholine, AK275, and benzyloxycarbony-Leu-Abu-CONH—CH2CH3, and derivatives thereof.

In one aspect, the calpain inhibitor comprises calpastatin or a calpastatin peptide mimetic. In one aspect, the calpain inhibitor binds to the calcium binding domain of calpain.

In one aspect, the calpain inhibitor that binds to a calpain calcium binding domain comprises: PD 150606, [3-(4-Iodophenyl)-2-mercapto-(benzyloxycarbonyl)-2-propenoic acid], and PD 1151746, 3-(5-fluoro-3-indolyl)-2-mercapto-(benzyloxycarbonyl)-2-propenoic acid, or any derivatives thereof.

In another aspect, the calpain inhibitor is a polypeptide comprising the amino acid sequence shown in SEQ ID NO: 44 or 45. In yet another aspect, the calpain inhibitor is a polypeptide comprising an amino acid sequence having at least 70% sequence identity to the amino acid sequence shown in SEQ ID NO: 44 or 45.

In one aspect, the calpain inhibitor is administered with one or more additional therapeutic agent(s), wherein the additional therapeutic agent(s) can be administered at the same or at a different time than the calpain inhibitor. The additional therapeutic agent can comprise, for example, an anti-bacterial substance, an anti-viral substance, an anti-inflammatory substance, a bronchodilatory substance, an antihistamine substance or an anti-tussive substance. The anti-viral substance can comprise, for example, ritonavir, saquinavir, indinavir, nelfinavir, amprenavir, or any derivative thereof.

In one aspect, the additional therapeutic agent comprises a steroid, a beta-2 agonist, a PDE4 inhibitor, a LTD4 antagonist, an anticholinergic agent, a corticosteroid, a steroid anti-inflammatory agent or a bronchodilator. In one aspect, the additional therapeutic agent comprises a chemokine receptor antagonist.

In one aspect, the leukocyte is a white blood cell, a neutrophil, a lymphocyte, a monocyte, a basophile, a macrophage, a dendritic cell, a mast cell, a phagocyte or an eosinophil, or any combination thereof.

In one aspect, the calpain inhibitor is administered orally, parenterally, by inhalation, intranasally, topically, subcutaneously, intramuscularly, rectally or by intrapulmonary injection.

In one aspect, the calpain inhibitor is linked to a targeting moiety that specifically binds to the surface of a cell in the pulmonary epithelium, a cell in the lower respiratory airway or a cell in the upper respiratory airway of the subject.

In still a further aspect, the method further comprises administering a calcium chelator to the subject. In yet another aspect, the method further comprises administering a calcium blocker to the subject.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. Epithelial cell junctions are affected by bacterial stimulation. Polarized 16HBE cells were incubated for 4 h with (FIG. 1A) media, (FIG. 1B) heat killed PAO1, (FIG. 1C) P3C or (FIG. 1D) thapsigargin. Cells were incubated with biotin (red) and stained for ZO-1 (green). Apical to basolateral x-y scans are shown with corresponding z-sections below. Data is representative of at least three separate experiments. For a color reproduction of this figure see Chun and Prince, TLR2 signaling stimulates calpain activity that targets epithelial junctions to accommodate PMN transmigration. Cell: Host Microbe 5:47, 2009, which is incorporated by reference in its entirety.

FIG. 2. Occludin and E-cadherin localization and barrier function in polarized 16HBE airway cells incubated with bacteria. (FIG. 2A and FIG. 2B) Localization of occludin (green, FIG. 2A) and E-cadherin (red, FIG. 2B) following 6 h incubation with media or heat killed PAO1. Both occludin and E-cadherin staining is reduced at cell junctions following h incunbation with PAO1. Cells were stained with DAPI (blue). FIGS. 2C-E: Epithelial barrier function is unchanged in response to heat killed PAO1. FIG. 2C: Transepithelial resistance was assessed at the indicated times following bacterial exposure. FIG. 2D: Paracellular permeability was measured after 5 h stimulation with heat killed PAO1 or P3C by adding Alexa Fluor 488 dextran (10,000 MW) apically for 1 h and monitoring basolateral fluorescence at ex 485, em 535. Data is represented as the percentage of fluorescence compared with cells treated with EGTA which disrupts the junctions completely. FIG. 2E: Bacterial transmigration across the monolayer was quantified by adding 2×10⁷ CFU live PAO1 apically for 1 h after pretreatment for 4 h with heat killed PAO1, P3C and thapsigargin or 1 h treatment with EGTA and monitoring PAO1 that reach the basal compartment of the Transwell. Data is represented as a percentage of inoculum and represents the mean±s.d. of quadruplicate samples of one representative of at least three separate experiments.

FIG. 3. Epithelial calpain and calpastatin expression. mRNA levels of (FIG. 3A) calpain 1, (FIG. 3B) calpain 2, (FIG. 3C) and calpastatin were determined in 1HAEo-cells stimulated with PAO1 or P3C by real-time PCR. Values were normalized to actin and are shown as the fold change in expression relative to the endogenous level in media alone treated cells. Data are mean±s.d. of duplicate samples of one representative from three independent experiments. (*P<0.05 compared with media alone controls; Student's t-test).

FIG. 4. TLR2 mediated activation of calpain. (FIG. 4A) 1HAEo-cells or (FIG. 4B) Human small airway epithelial cells in primary culture (SAEC) were loaded with a fluorescent calpain substrate, t-BOC L-leucine L-methionine, and incubated with heat killed PAO1 or P3C in the presence or absence of ALLN at the indicated times. FIG. 4C: 1HAEo-cells expressing scrambled or TLR2 siRNA were stimulated with heat killed PAO1 at the indicated times. The change in fluorescence was quantified with ex 360 nm, em 465 nm. Data represents the mean±s.d. of quadruplicate samples of one representative from three independent experiments. (*P<0.05 compared with media alone controls; Student's t-test). FIG. 4D: Human SAECs were loaded with the fluorogenic calpain substrate, t-BOC-Leu-Met, and incubated with heat-killed PAO1 or P3C in the presence of 20 mM calpeptin or DMSO vehicle control at the indicated times.

FIG. 5. Occludin associates with calpain at the membrane. FIG. 5A: Mobilization of calpain into membrane fractions isolated from 1HAEo-cells stimulated with heat killed PAO1 using a pan-calpain antibody. FIG. 5B: Increased co-localization of calpain (green) and occludin (red) in polarized 16HBE cells following 1 h stimulation with heat killed PAO1 or P3C. FIG. 5C: Immunoprecipitation of occludin from 1HAEo-cells following heat killed PAO1 stimulation and detection of occludin and calpain by immunoblot.

FIG. 6. Occludin is cleaved by calpain. FIG. 6A: Identification of occludin cleavage product by occludin immunoprecipitations (IP) and western blot (WB) from 1HAEo-cells and (FIG. 6B) human small airway epithelial cells in primary culture (SAEC) following incubation with media (M), with heat killed PAO1 (PAO) or P3C. FIG. 6C: Detection of occludin cleavage in 1HAEo-cells overexpressing TLR2 WT or TLR2 Y616A/Y761A (TLR2YY). FIG. 6D: Detection of occludin cleavage in cells treated with 6 μM BAPTA/AM, 20 μM ALLN, 20 μM GM6001, or 25 μM Z-DEVD-FMK. FIG. 6E: Detection of occludin cleavage in P3C stimulated scrambled control or calpain 1 and 2 siRNA (CAPN 1 & 2) expressing cells. Silencing of calpain 1 and 2 expressions in scrambled control and siRNA expressing cells is shown in the adjacent panel. Data are representative of at least three separate experiments. FIG. 6F and FIG. 6G: Detection of occludin cleavage in P3C stimulated scrambled control or calpain 1 and 2 siRNA (CAPN 1 & 2) expressing cells. Silencing of calpain 1 and 2 expressions in scrambled control and siRNA expressing cells is shown. Data are representative of at least three separate experiments.

FIG. 7. FIG. 7A: Colocalization of calpain (red) and E-cadherin (green) in polarized 16HBE cells following 1 hr stimulation with heat-killed PAO1, P3C, and thapsigargin (Thaps) is shown in xy and xz sections. Co-localization is increased following 1 hr stimulation with heat-killed PAO1, P3C, and thapsigargin (Thaps) relative to media alone control. The adjacent panel shows an enlarged version of the xy merged image. FIG. 7B: Immunoprecipitation (IP) of E-cadherin from 1HAEo-cells incubated with media alone (M), thapsigargin, or heat-killed PAO1 and detection of E-cadherin and calpain by immunoblot. FIG. 7C: Identification of the E-cadherin cleavage product in 1HAEo-cells following 4 hr stimulation with thapsigargin as compared with media (M) control. FIG. 7D: Detection of E-cadherin cleavage products in 1HAEo-cells following 4 hr stimulation with heat-killed PAO1 or P3C in the presence or absence of 20 mM calpeptin. Data are representative of at least three separate experiments. For a color reproduction of this figure see Chun and Prince, TLR2 signaling stimulates calpain activity that targets epithelial junctions to accommodate PMN transmigration. Cell: Host Microbe 5:47, 2009, which is incorporated by reference in its entirety.

FIG. 8. TLR2-mediated calpain activation in the airways contributes to PMN recruitment. The percentage of PMNs in a single cell suspension of lung was quantified in WT or tlr2−/− pups treated with i.p. calpeptin or vehicle (Un) and intranasally inoculated with 10⁸ CFU PAO1 or PBS. FIG. 8A: Myeloperoxidase activity was quantified in the apical compartment of 16HBE monolayers stimulated with heat killed PAO1 (HKPAO1) or P3C in the presence or absence of calpeptin. Results are presented as fold increase over media control. Data represents mean±s.d. of sextuplicate samples of one representative from three independent experiments. (P<0.01; Student's t-test). FIG. 8B: The percentage of PMNs in a single cell suspension of lung was quantified in WT or tlr2−/− pups treated with i.p. calpeptin or vehicle (Un) and intranasally inoculated with 10⁸ CFU PAO1 or PBS. FIG. 8C: Lung suspensions from representative mice in (A) were immunoblotted for occludin and E-cadherin cleavage products. FIG. 8D: Adult WT mice treated with i.p. calpeptin or vehicle (Un) were intranasally inoculated with 10⁹ CFU PAO1 or PBS for 2 hr, and BAL and lung cell suspensions were obtained and absolute numbers of PMNs enumerated by flow cytometry. For FIG. 8B and FIG. 8D, individual mouse values are shown, and the short horizontal lines indicate the median values of each group. (*p<0.05, **p<0.01 compared with WT PAO1 infected mice; nonparametric Mann-Whitney test.) FIG. 8E: Bacterial counts, in CFU/ml, were determined from whole-lung suspension. FIG. 8F: KC mRNA expression was quantified from lung suspensions and KC protein levels determined from BAL.

FIG. 9. Bacterial Induction of (FIG. 9A) Ca2+ fluxes and (FIG. 9B) subsequent NF-kB signaling in airway epithelial cells is TLR-2 dependent.

FIG. 10. TLR2 signaling activates calpains which cleave occludin, E-cadherin and Ezrin, facilitating PMN transmigration. Type II toxins target occludin and ezrin through GTPases and ADP ribosylating activities, facilitating bacterial invasion.

FIG. 11. Occludin and E-Cadherin Are Targets for Calpain Cleavage. FIG. 11A: Distribution of occludin (red) and E-cadherin (green) is altered following 6 hr incubation with media, heat-killed PAO1, or P3C. Cells were stained with DAPI (blue). Data are representative of at least three separate experiments. FIG. 11B: In the presence of 20 mM CaCl₂, 1 mg of exogenous human calpain 1 cleaved occludin; and E-cadherin, but not JAM-1 or claudin-1, immunoprecipitated from 1HAEo-cell lysates. The exogenous calpain 1 in these reactions is autolysed upon activation in the presence of Ca2+. * indicates shorter exposure time on the three right bands compared to the three left bands. Data are representative of at least three separate experiments. For a color reproduction of this figure see Chun and Prince, TLR2 signaling stimulates calpain activity that targets epithelial junctions to accommodate PMN transmigration. Cell: Host Microbe 5:47, 2009, which is incorporated by reference in its entirety.

FIG. 12. Increased colocalization of calpain (green) and occludin (red) in polarized 16HBE cells following 1 hr stimulation with heat-killed PAO1 or P3C (relative to media control) is shown in xy and xz sections. The adjacent panel shows an enlargement of the white boxed area of the xy image. For a color reproduction of this figure see Chun and Prince, TLR2 signaling stimulates calpain activity that targets epithelial junctions to accommodate PMN transmigration. Cell: Host Microbe 5:47, 2009, which is incorporated by reference in its entirety.

FIG. 13. FIG. 13A: Immunoprecipitation of occludin from 1HAEo-cells following stimulation with heat-killed PAO1 or P3C and detection of occludin, pan-calpain, calpain 1, and calpain 2 by immunoblot. Data are representative of at least three separate experiments. FIG. 13B: Immunoprecipitation of occludin from 1HAEo-cells following heat-killed PAO1 stimulation and detection of the 80 kDa hyperphosphorylated form of occludin, 60 kDa full-length form of occludin, and 45 kDa cleavage fragment of occludin. FIG. 13C: Detection of occludin cleavage in P3C-stimulated scrambled control or calpain 1 and 2 siRNA (CAPN 1 and 2)-expressing cells. Silencing of calpain 1 and 2 expressions in scrambled control and siRNA-expressing cells is shown in the adjacent panel. FIG. 13D: Neutravidin immunoprecipitation (IP) from biotinylated 1HAEo-cells that were transfected (Txf) with C-terminal myc6-tagged occludin (Occ myc6) or N-terminal RFP-tagged occludin (RFP Occ) and incubated with media alone (M) or heat-killed PAO1 (PAO1) were immunoblotted (WB) with anti-myc or anti-RFP antibodies. Cartoon illustrates the forms of occludin that correspond with the bands on the immunoblot. (Data are representative of at least three separate experiments).

FIG. 14. PMN Transmigration across Airway Epithelial Monolayer Is Facilitated by Calpain. FIG. 14A: The number of PMNs that have migrated into the apical compartment of 16HBE monolayers stimulated with heat-killed PAO1 or P3C in the presence or absence of calpeptin. FIG. 14B: The migration of 10⁶ calcein-AM labeled PMNs across a Transwell in response to 10⁸ CFU/ml live PAO1, 10⁸ CFU/ml HKPAO1, or 10 nM fMLP was not affected by the presence of 20 mM calpeptin. Data are presented as number of PMNs multiplied by 10⁴ and represent mean±SD of sextuplicate samples of one representative from three independent experiments. (p<0.05, p<0.001; Student's t test.)

FIG. 15. Calpeptin does not inhibit PMN myeloperoxidase activity. FIG. 15A: Myeloperoxidase activity of 0.0125 μg/ml recombinant human myeloperoxidase was not inhibited by 20 μM calpeptin. FIG. 15B: Similarly, myloperoxidase activity of 10⁴ PMNs was not inhibited when incubated with 20 μM calpeptin. Results are presented as fold increase over baseline. Data represents mean±s.d. of sextuplicate samples.

FIG. 16. PMN migration in response to live PAO1. Migration of PMN across polarized 16HBE monolayers treated with various concentrations of live PAO1. Result is represented as the number of PMNs multiplied by 10⁴. Data represents mean±s.d. of sextuplicate samples.

### DETAILED DESCRIPTION OF THE INVENTION

The issued patents, applications, and other publications that are cited herein are hereby incorporated by reference to the same extent as if each was specifically and individually indicated to be incorporated by reference.

The disclosure provides embodiments relating to compositions and methods for preventing, treating, reducing or ameliorating conditions or diseases involving an inflammation-related disease or disorder. In one embodiment, an inflammation-related disease or disorder comprises inflammation caused (at least in part) by leukocytes that migrate through the endothelium in order to arrive at a tissue or pulmonary site and cause inflammation. In one embodiment, the site of inflammation is in the pulmonary system. In one embodiment, the site of inflammation is in the gastrointestinal system (i.e., Crohn's disease, ulcers, etc.). In one embodiment, the invention described herein relates to compositions and methods that reduce calpain activity in a subject having or at risk of having an inflammation-related diseases or disorder. In another embodiment, the invention described herein relates to compositions and methods that reduce calpain activity in a subject having, or at risk of having an airway inflammation-related disease or disorder.

The methods described herein provide a method for reducing inflammatory responses in a subject, the method comprising administering a calpain inhibitor to a subject at an effective amount to reduce transmigration of a leukocyte, a white blood cell, a neutrophil, a lymphocyte, a monocyte, a basophile, a macrophage, a dendritic cell, a mast cell, a polymorphic nuclear cell (PMN), or an eosinophil through an epithelial layer. In various embodiments, the epithelial layer is a pulmonary epithelial layer. In some embodiments, the epithelial layer is an epithelial layer in the gastrointestinal tract.

The methods described herein provide a method for blocking transmigration of a leukocyte through a cell layer. In one embodiment, the cell layer is an epithelial layer. In another embodiment, the cell layer is a pulmonary epithelial layer. In another embodiment, administration of one or more calpain inhibitors blocks leukocyte transmigration through an epithelial layer by inhibiting cleavage of E-cadherin. In another embodiment, administration of one or more calpain inhibitors blocks leukocyte transmigration through an epithelial layer by inhibiting cleavage of occludin. In still a further embodiment, administration of one or more calpain inhibitors blocks leukocyte transmigration through an epithelial layer by inhibiting dissolution of cell junctions between cells in the epithelial layer.

The invention also provides methods for preventing and/or treating diseases or conditions associated with inflammation. The method comprises administering to a subject, who is in need of anti-inflammation prevention or which suffers from inflammation, an effective amount of a calpain inhibitor, wherein said amount is effective to inhibit inflammation.

Definitions

The singular forms “a,” “an,” and “the” include plural reference unless the context clearly dictates otherwise.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

The term “calpain” includes, but is not limited to, human calpain1 (mu-calpain) or human calpain2 (m-calpain).

The term “calpain inhibitor” refers to a biochemical or chemical compound which inhibits or reduces the activity of calpain or the expression of a calpain gene. A “calpain inhibitor” can inhibit or reduce calpain activity and inhibits or reduce expression of a calpain gene. Calpains that can inhibited by the methods and compositions described herein, include, but are not limited to a full-length calpain, a calpain homolog, a calpain variant, a calpain analog, a mutant calpain, a calpain fusion protein, or a calpain peptide mimetic. Exemplary calpain inhibitors include peptides, peptidomimetics, small molecules, compounds, agents, dominant negative mutants of calpain activity, ligand mimetics, antibodies (e.g., monoclonal, polyclonal or single chain Fv; intact or binding fragments thereof), or nucleic acids (e.g., RNA, DNA, antisense oligonucleotides, double stranded RNA oligonucleotides (RNAi) or DNA oligonucleotides (vectors) containing nucleotide sequences encoding for the transcription of shRNA molecules) or derivatives and analogs thereof. Further exemplary calpain inhibitors are disclosed herein.

Administration of the calpain inhibitors described herein can be performed according to any method known to one skilled in the art. For example, when administered to a subject, the administration step can comprise directly contacting airway epithelial cells with a calpain inhibitor. The administration step can also comprise transport or diffusion of the calpain inhibitor across one of more biological membranes in a cell. Many suitable methods for the delivery of a calpain inhibitor to a site in a subject are known in the art. These methods include, but are not limited to, administration of a calpain inhibitor to a subject, wherein the administration step comprises a step of delivering the calpain inhibitor into the airway of the subject by inhalation with the use of a nebulizer. One skilled in the art will also recognize that exist many different methods suitable for use with the methods described herein for delivery of a therapeutic compound, for example a calpain inhibitor, to a selected epithelial layer in a subject. For example, creams, gels, ointments, emulsions, solutions, elixirs, lotions, suspensions, tinctures, pastes, foams, aerosols, irrigations, sprays, nebulized formulation, suppositories, bandages, dermal patches or any other formulations suitable for topical administration or any other method of delivery, including, but not limited to those described herein, can be used can be used to deliver a calpain inhibitor in conjunction with the methods described herein.

As used herein, the term “leukocyte” includes, but is not limited to a white blood cell, a neutrophil, a lymphocyte, a monocyte, a basophile, a macrophage, a dendritic cell, a mast cell, a phagocyte or an eosinophil, or any combination thereof.

“Subject” refers to a mammal, e.g., a human, or to an experimental or animal or disease model. The subject can also be a non-human animal, e.g., a horse, cow, goat, or other domestic animal.

The methods described herein also provide methods for reducing inflammation in a mammal (e.g., human, horse, pig, goat, cow, dog, cat, rat, or mouse). As described herein, compositions containing one or more calpain inhibitors can be used to reduce inflammation in a mammal. In addition, methods describing the use of one or more calpain inhibitors can be used to inhibit leukocyte transmigration across an epithelium within a mammal. In some embodiments, the methods provided herein can be used to treat a mammal having an inflammation-related disease or disorder or a mammal at risk of having an inflammation-related disease or disorder.

As used herein, “sequence identity” means the percentage of identical nucleotide or amino acid residues at corresponding positions in two or more sequences when the sequences are aligned to maximize sequence matching, i.e., taking into account gaps and insertions. Identity can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to give the largest match between the sequences tested. Computer program methods to determine identity between two sequences include, but are not limited to, the GCG program package (Devereux, J., et al., Nucleic Acids Research 12(1): 387 (1984)), BLASTP, BLASTN, and FASTA (Altschul, S. F. et al., J. Molec. Biol. 215: 403-410 (1990) and Altschul et al. Nuc. Acids Res. 25: 3389-3402 (1997)). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990).

The term “variant” refers to polynucleotides or polypeptides of the invention modified at one or more base pairs, codons, introns, exons, or amino acid residues (respectively) yet still retain the ability to inhibit a calpain. Variants can be produced by any number of methods, including but not limited to, error-prone PCR, shuffling, oligonucleotide-directed mutagenesis, assembly PCR, PCR mutagenesis, in vivo mutagenesis, cassette mutagenesis, recursive ensemble mutagenesis, exponential ensemble mutagenesis, site-specific mutagenesis, gene reassembly, and any combination thereof.

In one aspect, the methods of the invention are useful for reducing, treating, managing, or preventing an inflammation-related disease or disorder in a subject. The methods of the invention are useful for reducing, treating, managing, or preventing an inflammation-related disease or disorder a subject that has undergone, is currently undergoing, or can in the future undergo other treatments. For example, the calpain inhibitors can be administered to individuals that have been administered other therapeutic agents, or that have undergone other therapies such as administration of antibiotics or surgery.

The methods described herein can be useful for the treatment of a subject suffering from, or at risk of suffering from an inflammatory disease or disorder. In one aspect, the methods described herein can be used to reduce or inhibit inflammation in a subject by blocking the transmigration of leukocytes between epithelial cells in an endothelium. In one embodiment, this migration can occur through the paracellular space between epithelial cells.

In one aspect, the methods described herein involve the use of calpain inhibitors to inhibit or reduce the dissolution of cell junctions between cells in an epithelial layer thereby inhibiting the ability of a leukocyte to transmigrate between cells of the epithelial monolayer. The epithelium can be any epithelium, including, but not limited to an airway epithelium, a pulmonary epithelium, or a gastrointestinal epithelium. In some embodiments of the invention, the epithelial monolayer can comprise epithelial cells in the airway (for example the lungs or the trachea) of a subject. In other embodiments, the epithelial cells can comprise epithelial cells in the gut (for example, the intestine or the colon) of a subject.

As described herein, calpain inhibitors can block the transmigration of leukocytes through an epithelium layer and be used to treat or prevent inflammatory conditions. In specific embodiments, the use of one or more calpain inhibitors, as described herein, can be useful for preventing or inhibiting the re-organization of cell junctions at epithelial cell contacts. For example, in one embodiment, any calpain inhibitor described herein can be administered to a subject having, or at risk of having an inflammatory condition in the airway. In one embodiment, administration of the one or more calpain inhibitors to a subject can block dissolution of tight junctions in the subject and inhibit transmigration of leukocytes across the airway epithelium into the airway lumen. In another embodiment, administration of one or more calpain inhibitors to a subject can block dissolution of adherens junctions in the subject and inhibit transmigration of leukocytes across the airway epithelium into the airway lumen. In still a further embodiment, administration of one or more calpain inhibitors to a subject will block dissolution of any cell junction acting as a barrier to leukocyte transmigration across the barrier and thus inhibit transmigration of leukocytes across the epithelial layer.

One skilled in the art will be aware that numerous types of cell junctions exist in epithelial monolayers (e.g. tight junctions or adherens junctions). One skilled in the art will also appreciate that such junctions can exist in a dynamic state and that monolayer comprising cells having partially formed junctions can be capable of inhibiting transmigration of a leukocyte across the epithelial monolayer. Cells in an epithelial layer can have fully formed cell junctions surrounding the cell, wherein the junction forms contacts with neighboring cells. An epithelial monolayer can also comprise cells that have partially formed cell junctions. For example, a cell in an epithelial monolayer may have one or more regions where a cell junction is in the process of forming or dissolving (e.g. a tight junction or an adherens junction). However, cell junctions in an entire epithelial need not undergo dissolution in order that a leukocyte be capable of transmigrating across the layer. Thus, one skilled in the art will recognize that a localized loss of tight junction integrity, or adherens junction integrity can be sufficient to permit leukocyte transmigration between cells.

One skilled in the art will also recognize that cell junctions need not exist as discreet entities. Different types of junctions may share one or more components. Different types of junctions can also overlap in their special arrangement. The epithelial layers described herein can comprise one or more cells having fully or partially formed adherens junctions without having detectable tight junctions with neighboring cells. The epithelial layers described herein can also comprise one or more cells having fully or partially formed tight junctions without having detectable adherens junctions with neighboring cells. It is also possible for the epithelial layers described herein to comprise one or more cells having partially formed adherens and partially formed tight junctions. Accordingly, there is no requirement that the calpain inhibitors described herein actually stabilize either a tight junction or an adherens junction. In one embodiment, administering a calpain inhibitor to a subject in need thereof can inhibit leukocyte transmigration across an epithelial layer by increasing the integrity of a tight junction, by stabilizing a tight junction, by promoting the formation of a tight junction or by any combination thereof, without affecting the integrity, stability or formation of an adherens junction. In another embodiment, administering a calpain inhibitor to a subject in need thereof can inhibit leukocyte transmigration across an epithelial layer by increasing the integrity of an adherens junction, by stabilizing an adherens junction, by promoting the formation of an adherens junction or by any combination thereof, without affecting the integrity, stability or formation of a tight junction.

The methods described herein relate in part to the use of a calpain inhibitor to cause a reduction or an inhibition of E-cadherin cleavage, occludin cleavage, or ezrin cleavage in a cell of a subject. In one aspect, the methods described herein can be used to inhibit leukocyte transmigration across an epithelial layer by inhibiting or reducing the ability of one or more calpain proteins of a cell in the epithelial layer to cleave one or more E-cadherin molecules in a cell of the epithelial monolayer. In one embodiment, inhibition of calpain activity can result in reduced E-Cadherin cleavage and enhanced adherens junction stability, integrity, formation or any combination thereof. In another embodiment, inhibition of calpain activity can result in reduced E-Cadherin cleavage and enhanced tight junction stability, integrity, formation, or any combination thereof. In still a further embodiment, inhibition of calpain activity can result in reduced E-Cadherin cleavage and/or enhanced tight junction stability, integrity or formation, and/or enhanced adherens junction stability, integrity or formation, or any combination thereof.

In another aspect, the methods described herein can be used to inhibit leukocyte transmigration across an epithelial layer by inhibiting or reducing the ability of one or more calpain proteins of a cell in the epithelial layer to cleave one or more Occludin molecules in a cell of the epithelial monolayer. In one embodiment, inhibition of calpain activity can result in reduced Occludin cleavage and enhanced adherens junction stability, integrity, formation, or any combination thereof. In another embodiment, inhibition of calpain activity can result in reduced Occludin cleavage and enhanced tight junction stability, integrity, formation, or any combination thereof. In still a further embodiment, inhibition of calpain activity can result in reduced Occludin cleavage and/or enhanced tight junction stability, integrity or formation, and/or enhanced adherens junction stability, integrity or formation, or any combination thereof.

In another aspect, the methods described herein can be used to inhibit leukocyte transmigration across an epithelial layer by inhibiting or reducing the ability of one or more calpain proteins of a cell in the epithelial layer to cleave one or more Ezrin molecules in a cell of the epithelial monolayer. In one embodiment, inhibition of calpain activity can result in reduced Ezrin cleavage and enhanced adherens junction stability, integrity, formation, or any combination thereof. In another embodiment, inhibition of calpain activity can result in reduced Ezrin cleavage and enhanced tight junction stability, integrity, formation, or any combination thereof. In still a further embodiment, inhibition of calpain activity can result in reduced Ezrin cleavage and/or enhanced tight junction stability, integrity or formation, and/or enhanced adherens junction stability, integrity or formation, or any combination thereof. Calpain inhibitors suitable for use with the methods described herein can include, but are not limited to peptides, peptidomimetics, small molecules and nucleic acids (e.g. siRNA molecules). Several classes of calpain inhibitors suitable for use with the methods described herein are known in the art, including, but not limited to, those calpain inhibitors described herein. In some embodiments the calpain inhibitors suitable for use with the methods described herein can be cell permeable. In other embodiments, non-cell permeable calpain molecules can be used in conjunction with the methods described herein. For example, in the case of non-cell permeable calpain inhibitors, a cell permeabilizing agent can be used to facilitate entry of the calpain inhibitor into a cell (e.g. an epithelial cell in an epithelial layer). Accordingly, agents, such as, for example liposomes, can be used to encapsulate one or more calpain inhibitors and can be used to facilitate delivery of the calpain inhibitors by fusion with the cell membrane of a target cell. In one aspect, the calpain inhibitor is administered in an amount sufficient to reduce epithelial junction or tight junction reorganization. In one aspect, the calpain inhibitor is administered in an amount sufficient to reduce leukocyte accumulation in the airway lumen in the subject. In one aspect, the calpain inhibitor is administered in an amount sufficient to reduce leukocyte accumulation in the gut lumen in the subject.

The invention described herein is based in part on the use of calpain inhibitors to reduce inflammatory responses. The invention described herein also provides specific compositions and methods that are useful for reducing inflammation in a subject. In other embodiments, the invention described herein provides specific compositions and methods that are useful for reducing inflammation responses by reducing leukocyte transmigration across airway epithelial layers.

One basis for the invention disclosed herein is that inhibition of calpain activity can block leukocyte transmigration across an epithelial layer. In one aspect, the invention described herein provides specific compositions and methods that can be useful for treating or preventing the occurrence of an airway inflammation-related disease or disorder in a subject. In other aspects, the invention described herein provides specific compositions and methods to reduce inflammatory responses in subject for the treatment or prevention of airway inflammation-related conditions such as those due to viral infections, bacterial infections, chronic obstructive pulmonary disease (COPD), asthma, pneumonia, cystic fibrosis or any combination thereof. In still other aspects, the invention described herein provides specific compositions and methods to reduce inflammatory responses any disease or condition associated with increased or excessive inflammation in any tissue.

A major function of epithelial signaling in response to bacteria in the airway lumen is the rapid accumulation of PMNs. While important for bacterial clearance, PMNs can themselves initiate oxidative damage, provide further proinflammatory stimulation and impede the ability of airway to provide a conduit for air exchange. Transmigration of PMNs from the vasculature and across the epithelial barrier into the airway lumen is an important step in the pathogenesis of inflammatory airway diseases and disorders (e.g., acute and chronic pneumonia). Epithelial signaling initiates the process of PMN recruitment, however the intensity of signaling can be dependent upon the types of organisms that are perceived, the molecular compositions of their pathogen associated molecular patterns (PAMPs) and the ligands that activate eukaryotic receptors. Opportunists that infect the lungs, such as Pseudomonas aeruginosa, actively regulate expression of these PAMPs as well as secreted virulence factors in response to the environment that they sense. Ventilator-associated pneumonia, often caused by P. aeruginosa and similar opportunists, has the highest case fatality rate of hospital acquired infections. These organisms not only colonize the airways, they can cause invasive infection and sepsis and can be responsible for substantial mortality. While some strains of P. aeruginosa are highly motile and express numerous secreted virulence factors and toxins, others adapt to the milieu in the human airway by down regulating toxin production. Nonetheless, even less virulent opportunists continue to elicit epithelial IL-8 production and PMN recruitment.

Accordingly, the methods described herein are useful in the treatment of inflammation-related disease or disorders, resulting, for example, in reduction of tissue damage, bronchial hyperreactivity, remodeling or disease progression.

TLR2 Signaling in the Airway Epithelium

The role of the mucosal epithelium in sensing and initiating immune responses to microbes is well accepted. Several classes of microbial receptors are present in airway cells. Among these receptors, the toll-like receptors (TLRs) transduce the signals provided by pathogen associated molecular patterns (PAMPs), shared bacterial components, such as LPS, cell wall lipoproteins and flagella. Although the genes for the major TLRs and their adaptors are conserved in airway cells, expression is variable. The distribution of specific TLRs, whether they are apical, baso-lateral or intracellular also determines their importance in signaling. TLR2, which responds to bacterial lipoproteins and cell wall components, is displayed within the context of a lipid raft on the apical surface of airway. The PAMPs that activate TLR2 are shed from growing organisms providing immuno stimulation even if the organisms are predominantly enmeshed in airway mucin. PAMPs are highly conserved components of bacteria, and are not only associated with pathogens. TLR2 is recruited into caveolin-1 associated lipid rafts in response to bacterial ligands This recruitment occurs along with a glycolipid co-receptor asialoGM1, a well characterized receptor for P. aeruginosa pili and flagella. Although P. aeruginosa actively sheds LPS during its growth, the LPS associated receptor, TLR4, is not abundant on the apical surface of airway cells nor is LPS a potent stimulus for epithelial activation. In contrast, ligation of epithelial TLR2 or the associated asialoGM1 rapidly activates MAPKs and NF-κB to generate the PMN chemokine IL-8, as well as proinflammatory cytokines and corresponding receptors. The IL-8 content of broncho-alveolar lavage fluid in CF has been validated as a clinical marker for the severity of airway inflammation in CF and IL-8 production can be used as an index of epithelial inflammation.

PMN Recruitment into the Airway

A major consequence of TLR2 signaling is the activation of IL-8 expression and mobilization of PMNs into the airway. The central role of the PMN in the immune response to P. aeruginosa infection is recognized. Animal models of P. aeruginosa pathogenesis use PMN depletion to facilitate infection and models of infection using macrophage depletion demonstrate no defect in P. aeruginosa clearance. In murine models where P. aeruginosa infection is introduced intranasally, PMNs are the predominant CD45+ white blood cells identified by flow cytometric analysis of a single cell suspension of the infected lung. They are also readily observed by histopathology is this system. The process of PMN transmigration from the vascular space across the endothelial cells following chemokine gradients has been described. Much less is known about the movement of PMNs across epithelial tight junctions and into the airway. Epithelial tight junctions can be selectively modulated to enable PMNs to access the airway. This process can be linked to the initial TLR2 cascade which functions to recruit phagocytes to the site of infection. Ca⁺ fluxes can also be involved in initiating signaling to modify the physiological properties of the junction.

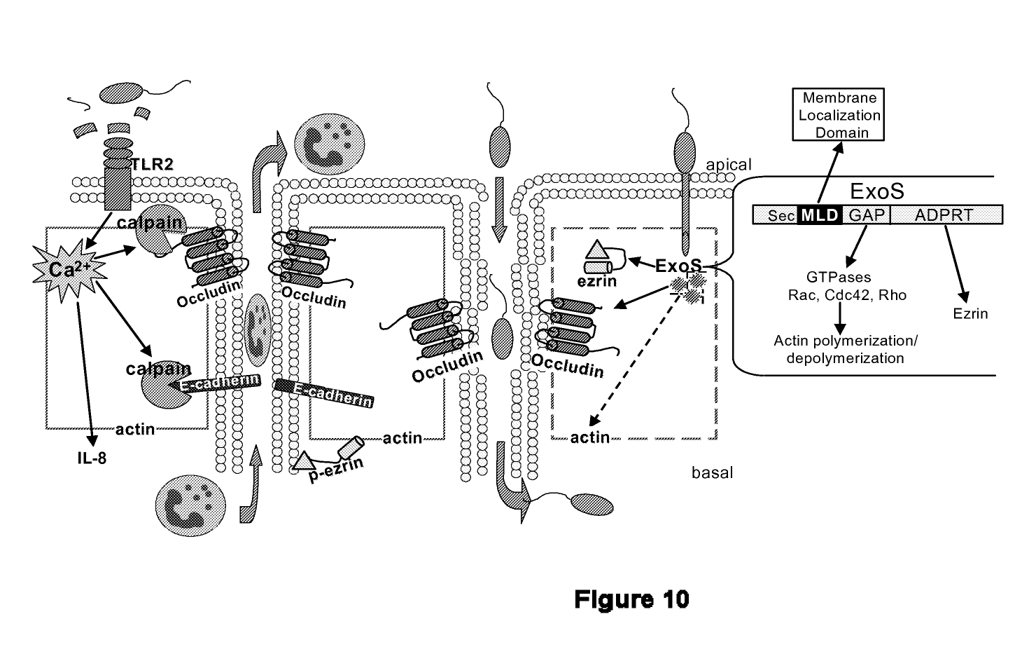

Epithelial junctions are composed of tight, adherens and gap junctions (FIG. 10). The tight junction is a semi-permeable barrier that causes the apical/baso-lateral polarization of epithelial cells and permits the selective diffusion of solutes and larger compounds across the epithelium. Some junctional components, such as occludin and E-cadherin span the paracellular space to establish homotypic contacts with adjacent cells. Others, for example ERM (ezrin, radixin, moesin) proteins, control the distensibility of the junctions by linking the plasma membrane and the actin cytoskeleton. Functional proteins that span the paracellular space, as well as the ERM linkers, can be affected by PMN migration. This effect can be determined by examining E-cadherin, occludin and ezrin in airway cells stimulated by P. aeruginosa or a TLR2 agonist.

E-cadherin is a single trans-membrane-spanning protein that forms homotypic interactions with the corresponding extracellular domains on adjacent cells in a Ca²⁻ dependent fashion. Intracellular domains anchor E-cadherin to the actin cytoskeleton via the catenins E-cadherin is important for the structural organization of the overlying epithelial tight junction and α-catenin links E-cadherin to ZO-1, a tight junction marker. E-cadherin trafficking plays a central role in cellular growth and development, maintenance of epithelial polarity, and Wnt signaling. Active E-cadherin endocytosis at the epithelial membrane can be regulated by the small GTPases Rac and Cdc42. E-cadherin is also a target of proteases, such as MMP-7, TNFα converting enzyme (TACE) (ADAM 17) and ADAM 10 which mediate the shedding of the extracellular domain of E-cadherin in damaged and apoptotic cells as well as fibroblasts.

Occludin, which has four transmembrane domains that form two extracellular loops of 44 and 45 amino acids, was one of the first tight junction proteins to be identified. It is not essential for epithelial barrier function, but is important in the regulation of paracellular permeability. Occludin null mice exhibit chronic inflammation in the gastric epithelium. In vitro expression knockdown experiments, as measured by changes in dextran permeability, transepithelial resistance (TER) and clearance of apoptotic cells, show a role for occludin in regulating paracellular permeability across the epithelium. A role for occludin in the regulation of PMN transmigration across MDCK cells exists but this activity does not map to domains within the extracellular loops.

IL-8 production and displacement of occludin to the cytoplasm occurs in endothelial cell as a consequence of viral infection. Occludin and the E-cadherin binding partner, α-catenin, are attached intracellularly to the apical tight junction protein ZO-1. Various functions of occludin can be regulated by phosphorylation or endocytosis in response to signaling from MAPKs, from oxidative stress, cell-cell adhesion and in response to changes in intracellular Ca⁺. Proteolytic cleavage of occludin can occur in response to exposure to the protease/allergen der p 1 and in response to hormones.

The ERM (ezrin, radixin and moesin) proteins link the plasma membrane to the actin cytoskeleton. These related proteins are associated with de-anchoring of actin and function to regulate cellular rigidity of T cells. Ezrin is highly expressed in the pulmonary epithelium and accumulates at the apical surface of these cells where it contributes to the formation of microvilli. In its deactivated state, the amino and C-termini of ezrin interact and prevent binding to the membrane or cytoskeleton. Ezrin is activated by PIP₂-stimulated conformational changes and phosphorylation of T567 and by any of several kinases. In the phosphorylated state, the termini of ezrin display binding sites for membrane components (e.g., CD44, ICAMs, EBP50), scaffold proteins and actin. The dephosphorylation of ezrin is involved in the coupling and uncoupling of the lipid rafts to mediate B cell activation and coordination of immune signaling. S100P Ca²⁺ binding protein also binds to ezrin to unmasking its F-actin binding site. This is consistent with the scheme that Ca²⁺ fluxes initiate changes in these cytoskeletal components.

Infectious Diseases and Infectious Agents

In some embodiments, the methods described herein can be used in the prevention, treatment, inhibition or reduction of inflammation in a subject suffering from a respiratory infection, or at risk of suffering from a respiratory infection. While the following infectious diseases and infectious agents are provided for purposes of example, the methods and compositions described herein can be used for treatment of infection.

Acute respiratory infections can affect both the upper or lower respiratory systems. An upper respiratory infection can involve the ears, nose, throat or sinuses. Examples of upper respiratory tract infections include the common cold (e.g., viral); the flu (e.g., influenza virus); otitis media, pharyngitis, acute sinusitis or chronic sinusitis, and tonsillitis, which involve inflammation of the middle ear, throat, sinuses, and tonsils, respectively. Lower respiratory infections can involve the trachea, bronchial tubes and the lungs themselves. Examples of lower respiratory tract infections include bronchitis and pneumonia. In a single infection, one or both of the upper and lower respiratory systems can be affected.

Respiratory tract infections can be of bacterial, viral, or fungal origin; although there are also rarer types, such as parasitic infections.

Pneumonia

In some embodiments, the methods described herein can be used in the prevention, treatment, inhibition or reduction of inflammation in a subject suffering from pneumonia, or at risk of suffering from pneumonia. While the following pneumonia-related infectious diseases and infectious agents are provided for purposes of example, the methods, compositions, and devices described herein can be used for treatment of pneumonia or a pneumonia-related condition.

Pneumonia is a condition is caused by a wide variety of bacteria, viruses, fungi, and other types of organisms that infect the respiratory tract. It can be defined as an inflammation of the lung tissue, whereby white cells in the lungs prevent the alveoli from functioning properly. Pneumonia infections afflict 150 million children a year worldwide and the incidence in Europe and the U.S. is 35/1000 under the age of 5 and 7/1000 in adolescence. The incidence increases significantly in the elderly. Total yearly sales of antibiotics are estimated to be over $25 billion globally, with sales in the US reaching almost $10 billion.

This condition can be life-threatening. Infectious agents can enter through the mouth and reach the lung during respiration. Smoking contributes to pneumonia since it damages the cilia lining the respiratory tract. Malnutrition or conditions like kidney failure or sickle cell disease also impair the lung's ability to get rid of microorganisms that cause pneumonia. Moreover, viral infections of the upper respiratory tract can predispose a person to pneumonia by also damaging the protective cilia.

Bacterial infection of the airways and the resulting inflammation is the first step in the pathogenesis of pneumonia; it is the fundamental process causing the chronic and ultimately fatal destruction of the lung in cystic fibrosis as well as the more acute but similarly devastating ventilator-associated pneumonias. The airway epithelium plays an active role in the defense against pulmonary infection as a fully functional component of the innate immune system. Activation of toll-like receptors, for example Toll-Like Receptor 2 (TLR2), presented on the surface of airway cells initiates the release of Ca²⁺ from cytoplasmic stores. These Ca²⁺ fluxes are both sufficient and necessary to activate MAPKs and NF-κB which initiate expression of chemokines, such as IL-8, that recruit PMNs into the airway. Activation of TLR2 signaling activates the Ca²⁺ dependent protease calpain, which targets epithelial junctional proteins to facilitate paracellular passage of PMNs into the airway (FIG. 10). These same junctional components are also the targets of bacterial virulence factors, the type III secreted toxins of P. aeruginosa that further modify the epithelial barrier to facilitate bacterial invasion.

Among children 12 and under, the most frequent cause of pneumonia is the pneumococcus bacterium. Among adolescents and young adults, the most frequent infective agent is a bacteria-like microbe called Mycoplasma pneumoniae.

Bacterial pneumonia can also ensue as a complication of influenza A; secondary infections are most often caused by Streptococcus pneumoniae, Haemophilus influenzae, or (most serious of all) Staphylococcus aureus.

Bacteria that have been associated with various pneumonias include, Streptococcus pneumoniae, Streptococcus pyogenes (Grp A), Streptococcus agalactiae (Grp B), Staphylococcus aureus, Bacillus anthracis, Other Bacillus sp., Nocardia sp., Enterobacteriaceae, Pseudomonas aeruginosa, Acinetobacter sp., Burkholderia pseudomallei, Burkholderia mallei, Yersinia pestis, Francisella tularensis, Hemophilus influenzae, Bordetella pertussis, Neisseria meningitides, Legionella pneumophila, Legionella-like bacteria, Bacteroides melaninogenicus, Fusobacterium nucleatum, Peptostreptococcus sp., Peptococcus sp., Actinomyces sp., Mycobacterium tuberculosis, Other Mycobacterium sp., Mycoplasma pneumoniae, Branhamella catarrhalis, Chlamydia trachomatis, Chlamydia psittaci, Chlamydia pneumoniae, and Coxiella burnetii (Q-fever).

Viruses that have been associated with various pneumonias include, Influenza Parainfluenza, Cytomegalovirus, Adenovirus, Epstein-Barr Virus, Herpes Simplex Virus, Varicella-Zoster, Coxsackievirus, Measles, Rhinovirus, and Respiratory Syncytial Virus. Fungi that have been associated with various pneumonias include Aspergillus sp., Mucorales sp., Candida sp., Histoplasma capsulatum, Blastomyces dermatitidis, Cryptococcus neoformans, Coccidioides immitis, Paracoccidioides brasiliensis, and Pneumocystis carinii. Protozoa that have been associated with various pneumonias include Plasmodium falciparum, Entamoeba histolytica, Toxoplasma gondii, and Leishmania donovani. Nematodes that have been associated with various pneumonias include Ascaris lumbricoides, Toxocara sp. and Ancyclostoma duodenale. Cestodes that have been associated with various pneumonias include Echinococcus granulosus.

Other Exemplary Types of Infectious Agents

In some embodiments, the methods described herein can be used in the prevention, treatment, inhibition or reduction of inflammation in a subject suffering from viral or bacterial infection, or at risk of suffering from viral or bacterial infection. While the following infectious diseases and infectious agents are provided for purposes of example, the methods, and compositions described herein can be used for treatment of inflammation due to any viral infection, bacterial infection or inflammation-related condition described herein.

Pulmonary tuberculosis (TB) is an example of a contagious bacterial infection caused by Mycobacterium tuberculosis. The lungs are primarily involved, but the infection can spread to other organs. TB is one of the most clinically significant infections worldwide, with an incidence of 3 million deaths and 10 million new cases each year. With improved sanitary conditions and the advent of antimicrobial drugs, the incidence of mortality had been steadily declining However, in most developed countries, there has been a resurgence of TB infection, in part due to immuno-compromised individuals (e.g., HIV-positive) and the emergence of multidrug-resistant (MDR) strains of M. tuberculosis.

Candida and Aspergillus are fungal respiratory tract infections, tending to appear in immuno-compromised subjects, such as transplant recipients. While Candida mainly infests the upper tracheobronchial tree with only an occasional chance of dissemination, Aspergillus has the potential to involve the deeper parenchyma. Other potential fungal pathogens include Cryptococcus, Pseudallerscheria and Coccidioides.

Mycobacterium tuberculosis is an intracellular pathogen that infects macrophages. Most inhaled bacilli are destroyed by activated alveolar macrophages. However, the surviving bacilli can multiply in macrophages and be released upon cell death, which signals the infiltration of lymphocytes, monocytes and macrophages to the site. Lysis of the bacilli-laden macrophages can be mediated by delayed-type hypersensitivity (DTH) and results in the development of a solid caseous tubercle surrounding the area of infected cells. Continued DTH causes the tubercle to liquefy, thereby releasing entrapped bacilli. The large dose of extracellular bacilli triggers further DTH, causing damage to the bronchi and dissemination by lymphatic, hematogenous and bronchial routes, and eventually allowing infectious bacilli to be spread by respiration. Anti-infective agents that are used to treat tuberculosis include, for example, isoniazid, rifampin, pyrazinamide, ethambutol, and streptomycin. Chemoprophylaxis can be effective and can consist of isoniazid at a dose of 300 mg/day for 6 to 9 months for adults. For children, the dosage can be 10 mg/kg/day, up to 300 mg, given as a single morning dose.

Pseudomonas aeruginosa causes chronic respiratory infections and is the leading cause of high morbidity and mortality in cystic fibrosis (CF). The initially colonizing P. aeruginosa strains are nonmucoid, but in the lung of a CF subject they begin to produce mucoid, which leads to the inability of subjects to clear the infection, even under aggressive antibiotic therapies. The emergence of the mucoid form of P. aeruginosa can be associated with further disease deterioration and poor prognosis. P. aeruginosa is also the second most common cause of infections in intensive care units, and a frequent cause of pneumonias. HIV-infected subjects are also at risk. Several penicillins, including ticarcillin, piperacillin, mezlocillin, and azlocillin, are active against Pseudomonas. Other anti-infective agents include, for example, ceftazidime, cefepime, aztreonam, imipenem, meropenem, and ciprofloxacin. Ticarcillin can be used at dosages of 16 to 20 g/day IV. Piperacillin, azlocillin, cefepime, ceftazidime, meropenem, and imipenem are active in vitro against some strains resistant to ticarcillin.

Bacillus anthracis, the causative agent of anthrax, is a large, Gram-positive, facultatively anaerobic, encapsulated rod. The spores resist destruction by disinfectants and heat and remain viable in soil and animal products for decades. Human infection can occur through the skin, in the gastro-intestinal tract, and inhalation of spores can result in fatal pulmonary anthrax. An anthrax vaccine, composed of a culture filtrate, is available for those at high risk (armed forces personnel, veterinarians, laboratory technicians, employees of textile mills processing imported goat hair). Repeated vaccination can be important to ensure protection and local reactions to the vaccine itself can occur.

Most strains of anthrax are susceptible to penicillin. However, the organism often manifests inducible beta-lactamases, so single-drug therapy with penicillin or cephalosporins is not recommended. Prophlaxis upon exposure requires oral ciprofloxacin 500 mg bid, or doxycycline 100 mg bid for 60 days; or amoxicillin 500 mg tid. Pulmonary anthrax is frequently fatal, but survival can occur with early treatment and intensive pulmonary and circulatory support. Corticosteroids can be useful but have not been adequately evaluated.

Picornaviruses, especially rhinoviruses and certain echoviruses and coxsackieviruses, cause the common cold, defined as an acute, viral infection of the respiratory tract, with inflammation in airways, including the nose, paranasal sinuses, throat, larynx, and sometimes the trachea and bronchi.

Immunity is specific for viruses by serotype or strain, and thus immunity against one strain is not protective against subsequent infection with another strain. Although effective experimental vaccines have been developed for some rhinoviruses, adenoviruses, and paramyxoviruses, no commercial vaccine is yet available. Prophylactic interferon offers promise in subjects at risk for morbidity from colds due to other complications, such as asthma or bronchitis. Interferon-alpha given intranasally limits acquisition of rhinovirus or coronavirus infection and reduces viral shedding; but can cause nasal inflammation with bleeding after prolonged exposure.

Influenza viruses (orthomyxoviruses) cause influenza, defined as an acute viral respiratory infection with influenza, a virus causing fever, coryza, cough, headache, malaise, and inflamed respiratory mucous membranes. Influenza produces widespread sporadic respiratory illness during fall and winter every year in temperate climates, often in focused single serotype epidemics, most often caused by influenza A (H3N2) viruses. Influenza B viruses can cause mild respiratory disease but can cause significant morbidity and mortality during an epidemic.

Exposure to influenza virus by natural infection or by immunization results temporarily in resistance to reinfection with the same virus type. Vaccines that include the prevalent strains of influenza viruses reduce the incidence of infection among vaccines when the HA and/or NA of the immunizing and infecting strains match. Anti-infective agents for influenza A types include amantadine and rimantadine, at 100 mg po bid. Amantadine and rimantadine can cause nervousness, insomnia, or other CNS side-effects, and drug resistance frequently occurs.

Severe acute respiratory syndrome (SARS) is a viral respiratory tract infection, first detected in China in late 2002. The viral agent has been identified as a previously unrecognized human coronavirus, called SARS-associated coronavirus (SARS-CoV). SARS is also an example of both upper and lower respiratory tract involvement caused by infection with a single organism. Early symptoms include runny nose and sore throat, which are then followed by dyspnea and dry cough, and can develop into adult respiratory distress syndrome requiring intervention with mechanical ventilation. Severe acute respiratory syndrome (SARS) has been recently shown to be associated with a coronavirus, SARS-CoV. Other pathogens can also have a role in some cases of SARS.

The Centers for Disease Control and Prevention currently recommends that subjects with SARS receive the same treatment that can be used for any subject with serious community-acquired atypical pneumonia. At present, the most efficacious treatment regimen, if any, is unknown. In several locations, therapy has included antivirals, such as oseltamivir or ribavirin. Steroids also have been given orally or intravenously to subjects in combination with ribavirin and other antimicrobials. Ribavirin does not inhibit virus growth or cell-to-cell spread of one isolate of the coronavirus tested.

The parainfluenza viruses are paramyxoviruses types 1, 2, 3, and 4 are closely related viruses causing many respiratory illnesses varying from the common cold to influenza-like pneumonia, with febrile croup as the most common severe manifestation.

Adenoviruses are a group of many viruses, some of which cause acute febrile disorders characterized by inflammation of the respiratory and ocular mucous membranes and hyperplasia of submucous and regional lymphoid tissue. Adenovirus infection related acute febrile respiratory disease is a manifestation of symptomatic adenoviral infection in children. Adenovirus infection related acute respiratory disease (ARD) has been observed in military recruits during periods of troop mobilization.

Vaccines containing live adenovirus types 4 and 7 have markedly reduced ARD in military populations; however, they are neither recommended nor available for civilian use. Vaccines for a few other serotypes have been developed but are not commercially available.

A special category of subjects, specifically lung transplant recipients are subject to many additional infectious agents. Cytomegalovirus is the most common viral infection, and a major cause of morbidity. Adenovirus infections have been reported, manifesting as an acute bronchitis/bronchiolitis to diffuse alveolar damage. Epstein Barr virus produces varied manifestations ranging from mononucleosis-like syndrome to posttransplant lymphoproliferative disorder. Pneumocystis carinii pneumonia often occurs due to depressed cellular immunity. Other miscellaneous infections include Pseudallerscheria boydii that mimics aspergillosis; nocardia, with manifestations including bronchopneumonia, abscess formation, cavitation, and empyema; Legionella pneumonia; and Toxoplasma gondii.

Asthma

In some embodiments, the methods described herein can be used in the prevention, treatment, inhibition or reduction of inflammation in a subject suffering, or at risk of suffering from asthma, an asthmatic condition or an exacerbation of an asthmatic condition. Asthma is a chronic inflammatory disease of the small airways in which the airways become blocked or narrowed. These effects can be temporary and reversible, but they cause shortness of breath, breathing trouble, and other symptoms. An asthma episode can be triggered by elements in the environment. These triggers vary from person to person, but common ones include cold air; exercise; allergens such as dust mites, mold, pollen, animal dander or cockroach debris; and some types of viral infections.

When the airways come into contact with an asthma trigger, the tissue inside the bronchi and bronchioles becomes inflamed. At the same time, the muscles on the outside of the airways constrict, causing them to narrow. A thick fluid (mucus) enters the airways, which become swollen. The breathing passages are narrowed still more, and breathing can be hampered.

Asthma symptoms can include one or more of the following: wheezing, coughing, shortness of breath, and tightness in the chest. These symptoms are especially indicative of asthma if they are recurrent. Episodes of these symptoms, especially coughing, that worsen at night are strongly indicative of asthma.

Asthma pathogenesis favors a role of Th2 cells and eosinophils. Characteristics of asthma include mononuclear, eosinophil and mast cell infiltration of the submucosa and submucosal remodeling, including fibrosis and neovascularization. Viral upper respiratory infections have been associated with 80% of asthma exacerbations in children and 50% of asthma episodes in adults. Human Rhinovirus has been implicated as the most common virus associated with asthma episodes. Although a controversial topic, viruses can play a role in the development of asthma. Disease exacerbations can arise from stimuli that are allergenic.

Chemokines, especially eotaxin and the monocyte chemoattractant proteins, are potent eosinophil chemoattractants and histamine releasing factors, making them important in generating an allergic inflammation. These chemokines can be the main histamine-releasing factors in the absence of antigen and IgE antibody. Th2 cells regulate the production of IgE, and the growth and differentiation of mast cells, basophils, and eosinophils, the primary players in the allergic response.

An asthma attack or exacerbation results from an acute narrowing of the bronchial airway in a subject. Three main factors can contribute to such a narrowing. The first factor can be bronchial inflammation. Inflammation can occur in response to an allergen or irritant that causes bronchial tubes to become red, irritated, and swollen. Inflamed tissues produce fluids that can accumulate and clog the smaller airways. Inflammation can cause tissue damage that leads to sloughing of damaged tissue into the airways, thus further narrowing the airway. The second factor can be bronchospasm or tightening of muscles around the bronchial tubes that causes constriction of the bronchial airway. The third factor can be hyper-reactivity or hypersensitivity to triggers such as allergens, irritants, and infections. Exposure to triggers can cause or aggravate the inflammation and constriction of the airway. During an asthma attack, a subject displays a reduced capacity to exhale, which can be assessed using, e.g., the peak expiratory flow rate (PEFR). PEFR measures a value called the peak flow number. During non-severe asthma attacks, peak flow numbers can be in the caution or danger range (e.g., 50% to 80% of personal best). In one embodiment, the invention provides methods for the use of calpain inhibitors to reducing inflammation in a subject suffering for, or at risk of suffering from an asthma exacerbation.

Asthma can be classified according to the severity of symptoms in a subject. Severe persistent asthma can be characterized by continuous symptoms of asthma, frequent exacerbations, FEV1 or PEFR less than about 60% of predicted value, and PEFR variability of greater than 30%. Moderate persistent asthma can be characterized by daily symptoms, exacerbations more than twice weekly, FEV1 or PEFR from 60% to 80% of predicted value, and PEFR variability greater than 30%. Mild persistent asthma can be characterized by the occurrence of symptoms more than twice weekly (yet less frequently than daily), FEV 1 or PEFR greater than 80% of predicted values, and a PEFR variability from about 20 to 30%. Mild intermittent asthma can be characterized by symptoms that occur, at most, twice weekly, FEV1 or PEFR greater than 80% of predicted values, and a PEFR variability less than 20%.

Current treatment includes bronchodilators, anti-inflammatory medications (including anti-leukotrienes) and, recently, an anti-IgE treatment. Bronchodilators provide relief from asthma by relaxing the muscles in the air tubes. Anti-inflammatory medications work to keep the air tubes open to prevent an asthma attack. The allergen bound to IgE activates mast cells and basophils that release the chemical mediators (histamines, leukotrienes and prostaglandins) that produce the allergic response. Use of an anti-IgE antibody to bind and thus sequester IgE helps reduce the allergic response by preventing the IgE from binding to mast cells and basophils.

Chronic Obstructive Pulmonary Disease

In some embodiments, the methods described herein can be used in the prevention, treatment, inhibition or reduction of inflammation in a subject suffering, or at risk of suffering from chronic obstructive pulmonary disease (COPD). COPD can be used to describe airflow obstruction that can be associated mainly with emphysema and chronic bronchitis. In both diseases, there can be chronic obstruction of the flow of air through the airways and out of the lungs, and the obstruction can be permanent and progressive over time. Emphysema causes irreversible lung damage by weakening and breaking the air sacs within the lungs. Elasticity of the lung tissue can be lost, causing airways to collapse and obstruction of airflow to occur. Chronic bronchitis is an inflammatory disease that begins in the smaller airways within the lungs and gradually advances to larger airways. It increases mucus in the airways and increases bacterial infections in the bronchial tubes, which, in turn, impedes airflow.

It is estimated that 10% of individuals above the age of 40 suffer from COPD and in 2020, COPD is predicted to the third leading cause of death in the developed world. Current treatment efforts involve the use of bronchodialators and corticosteroids.

Exacerbations of COPD are a major cause of morbidity and mortality. Etiological factors for exacerbations are bacterial infections, viral infections and pollutants. Airway obstruction in COPD subjects can make these individuals more susceptible to the infections. Approximately 50% of COPD subjects who have an exacerbation also have a bacterial infection. The most common bacterial infections are Haemophilus influenza and Streptococcus pneumonia. Viral infections are associated with 23-45% (more in the winter months) of subjects hospitalized with an exacerbation. Bacterial infections also exist in COPD subjects who are stable, but they are about twice as common in subjects who have an exacerbation. It has been demonstrated that subjects improve more quickly when treated with antibiotics, especially those with the most symptoms. In one embodiment, the invention provides methods for the use of calpain inhibitors to reducing inflammation in a subject suffering for, or at risk of suffering from COPD.

Long-term smoking is the most frequent cause of COPD. It accounts for 80 to 90 percent of cases. A smoker can be 10 times more likely than a non-smoker to die of COPD. The symptoms of COPD include: chronic cough, chest tightness, shortness of breath, an increased effort to breathe, increased mucus production, and frequent clearing of the throat.

The clinical development of COPD can be described in three stages, as defined by the American Thoracic Society: Stage 1: Lung function (as measured by FEV1 or forced expiratory volume in one second) is greater than or equal to about 50 percent of predicted normal lung function. There is minimal impact on health-related quality of life. Symptoms can progress during this stage, and subjects can begin to experience severe breathlessness, requiring evaluation by a pulmonologist. Stage 2: FEV1 lung function is about 35 to about 49 percent of predicted normal lung function, and there is a significant impact on health-related quality of life. Stage 3: FEV1 lung function is less than about 35 percent of predicted normal lung function, and there is a profound impact on health-related quality of life.

In one embodiment, the invention provides methods for the use of calpain inhibitors to reducing inflammation in a subject having about 50 percent of predicted normal lung function. In another embodiment, the invention provides methods for the use of calpain inhibitors to reducing inflammation in a subject having about 35 to about 49 percent of predicted normal lung function. In still another embodiment the invention provides methods for the use of calpain inhibitors to reducing inflammation in a subject having less than about 35 percent of predicted normal lung function.

In addition to smoking cessation, depending upon the severity of the disease, treatments can include bronchodilators that open up air passages in the lungs, anti-inflammatory medications, antibiotics, expectorants to help loosen up and expel mucus secretions, and exercise to strengthen muscles. People with COPD can eventually require supplemental oxygen and, in the end-stages of the disease, can have to rely on mechanical respiratory assistance.

In addition, other medications can be prescribed to manage conditions associated with COPD. These can include: diuretics, which are given as therapy to avoid excess water retention associated with right-heart failure, which can occur in some COPD subjects and digitalis (e.g., in the form of digoxin), which strengthens the force of the heartbeat. It is used with caution in COPD subjects, especially if their blood oxygen tensions are low, since they become vulnerable to arrhythmia when taking this drug; Painkillers, cough suppressants, and sleeping pills. Lung transplantation is being performed in increasing numbers and can be an option for people who suffer from severe emphysema. Additionally, lung volume reduction surgery has shown promise and is being performed with increasing frequency.

Other Inflammation-Related Disease or Disorders and Conditions

In some embodiments, the methods described herein can be used in the prevention, treatment, inhibition or reduction of inflammation in a subject suffering, or at risk of suffering from a variety of inflammatory or obstructive airway diseases and conditions. Other inflammatory or obstructive airway diseases and conditions to which the invention described herein can be applicable include cystic fibrosis, acute lung injury (ALI), acute/adult respiratory distress syndrome (ARDS), chronic obstructive pulmonary, airways or lung disease (COPD), including chronic bronchitis or dyspnea associated therewith, emphysema, as well as exacerbation of airways hyperreactivity consequent to other drug therapy and other inhaled drug therapy. The invention can be also applicable to the treatment of bronchitis of whatever type or genesis including, e.g., acute, arachidic, catarrhal, croupus, chronic or phthinoid bronchitis. Further inflammation-related disease or disorders to which the invention described herein can be applicable include pneumoconiosis (an inflammatory, occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis.

Other inflammatory conditions or diseases which can be treated with the methods disclosed herein are: (a) autoimmune stimulation (autoimmune diseases), such as lupus erythematosus, multiple sclerosis, infertility from endometriosis, type I diabetes mellitus including the destruction of pancreatic islets leading to diabetes and the inflammatory consequences of diabetes, including leg ulcers, Crohn's disease, ulcerative colitis, inflammatory bowel disease, osteoporosis and rheumatoid arthritis, psoriatic arthritis, ankylosing spondylitis; (b) diseases and conditions of the eye such as conjunctivitis, keratoconjunctivitis sicca, and vernal conjunctivitis, ocular inflammation associated with corneal ulcers, giant papillary conjunctivitis, blepharitis, chelazion, uveitis, dry eye, post-surgical inflammation, and contact lens associated inflammation; (c) allergic diseases such as hay fever, rhinitis, seasonal allergic conjunctivitis, vernal conjunctivitis and other eosinophil-mediated conditions; (d) skin diseases such as psoriasis, contact dermatitis, eczema, infectious skin ulcers, open wounds, and cellulitis, atopic dermatitis, alopecia areata, erythema multiforma, dermatitis herpetiformis, scleroderma, vitiligo, hypersensitivity angiitis, urticaria, bullous pemphigoid, lupus erythematosus, pemphisus, epidermolysis bullosa acquisita, and other inflammatory or allergic conditions of the skin; (e) infectious diseases including sepsis, septic shock, encephalitis, infectious arthritis, endotoxic shock, gram negative shock, Jarisch-Herxheimer reaction, shingles, toxic shock, cerebral malaria, bacterial meningitis, acute respiratory distress syndrome (ARDS), lyme disease, and HIV infection; (f) wasting diseases such as cachexia secondary to cancer and HIV; (g) inflammation due to organ, tissue or cell transplantation (e.g., bone marrow, cornea, kidney, lung, liver, heart, skin, pancreatic islets) including transplant rejection, and graft versus host disease; (h) adverse effects from drug therapy, including adverse effects from amphotericin B treatment, adverse effects from immunosuppressive therapy, e.g., interleukin-2 treatment, adverse effects from OKT3 treatment, adverse effects from GM-CSF treatment, adverse effects of cyclosporine treatment, and adverse effects of aminoglycoside treatment, stomatitis, and mucositis due to immunosuppression; (i) cardiovascular conditions including circulatory diseases induced or exasperated by an inflammatory response, such as ischemia, atherosclerosis, peripheral vascular disease, restenosis following angioplasty, inflammatory aortic aneurysm, vasculitis, stroke, spinal cord injury, congestive heart failure, hemorrhagic shock, ischemia/reperfusion injury, vasospasm following subarachnoid hemorrhage, vasospasm following cerebrovascular accident, pleuritis, pericarditis, and the cardiovascular complications of diabetes; (j) dialysis, including pericarditis, due to peritoneal dialysis; (k) gout; (l) chemical or thermal-induced inflammation due to burns, acid, alkali and the like; diseases of the bone and joints including rheumatoid and systemic sclerosis, and other diseases such as cystic fibrosis, pulmonary hypertension, myasthenia gravis, hyper IgE syndrome and acute and chronic allograft rejection, e.g., following transplantation of heart, kidney, liver, lung or bone marrow.

Having regard to their anti-inflammatory activity and in relation to inhibition of eosinophil activation, the methods described herein are also useful in the treatment of eosinophil related disorders, e.g., eosinophilia, in eosinophil related disorders of the airways (e.g., involving morbid eosinophilic infiltration of pulmonary tissues) including hyper-eosinophilia as it effects the airways and/or lungs as well as, for example, eosinophil-related disorders of the airways consequential or concomitant to Loffler's syndrome, eosinophilic pneumonia, parasitic (metazoan) infestation (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction.

Other Conditions or Disorders

Additionally, calpain inhibitors can be used to treat conditions that make a subject susceptible to airway infections. For example, in cystic fibrosis the respiratory tract is severely affected and populated by pathogenic bacteria leading to frequent infections. Cystic fibrosis is the most common autosomal recessive disease among Caucasian populations (1/2500 births). The respiratory tract can be severely affected and populated by pathogenic bacterial early in life an lead to frequent infections. Thus, in some embodiments, calpain inhibitors are administered to subjects afflicted with cystic fibrosis in order to treat or prevent inflammation caused by pathogen infection.

Calpain activity can also contribute to the pathophysiology of many different neurodegenerative disease, for example Amyotrophic lateral sclerosis (ALS), nerve injury, Parkinson's disease and Alzheimer's disease. In one embodiment, the methods and compositions described herein can be used for treating a subject having or at risk of having a condition associated with neurological damage. Conditions associated with neurological damage include, but are not limited to Amyotrophic lateral sclerosis (ALS), nerve injury, Parkinson's disease, Alzheimer's disease, cerebral ischemia, cerebral infarction, cerebral vasospasm, traumatic head injury, traumatic spinal cord injury, hemorrhage (such as subarachnoid hemorrhage, cerebral hemorrhage or aneurysmal hemorrhage), asphyxia (e.g., perinatal asphyxia), cardiac arrest, cardiac infarction, hypoxia or anoxia (e.g., from drowning, suffocation, anesthesia administered during surgical procedures, pulmonary surgery, cardiac bypass, or use of a heart-lung machine), hypoglycemia, reperfusion injury, a progressive pathological condition, Alzheimer's Disease, Parkinson's Disease, Huntington's Disease, Multiple Sclerosis, HIV-related neurodegeneration, cerebellar degeneration, seizure, glioblastoma, polyneuropathy, hydrocephalus, encephalitis, meningitis, epilepsy and schizophrenia.

The invention also provides methods for treating conditions that rely in some manner upon the cellular migration through an endothelium via reorganization of epithelial junctions in the paracellular space. For example, in one embodiment, the invention provides methods to treat or inhibit cancer metastasis by administering to a subject a calpain inhibitor. A rationale behind such a method is that for certain tumors, metastatic cells need to transmigrate through tissues or endothelia in order to reach the circulatory system and populate other tissue sites. By blocking transmigration, calpain inhibitors can be used to prevent, treat, or at least slow-down the rate of metastasis. Thus, calpain inhibitors can be used in combination with anti-cancer agents not only to treat metastatic tumors but also to prevent metastasis.

Prophylactic Treatments

Prophylactic efficacy in the treatment of an eosinophil related disorder, an inflammatory or obstructive airway disease and condition, COPD, asthma, an asthmatic condition or an exacerbation of an asthmatic condition, a viral or bacterial infection, a pneumonia, a respiratory infection or an inflammation related disease or disorder can be evidenced by reduced frequency or severity of symptomatic attack, e.g., improvement in lung function or improved airways hyperreactivity. Accordingly, the methods described herein are useful in the prophylactic treatment of inflammation related conditions of disorders, resulting, for example, in reduction of tissue damage, bronchial hyperreactivity, remodeling or disease progression.

The effectiveness of prophylactic treatments can be evidenced by a reduced requirement for other symptomatic therapy, e.g. therapy for or intended to restrict or abort symptomatic attack when it occurs, for example anti-inflammatory (e.g., corticosteroid) or bronchodilatory. For example, prophylactic benefit in asthma can be apparent in subjects prone to “morning dipping”. Morning dipping is a recognized asthmatic syndrome, common to a substantial percentage of asthmatics and characterized by asthma attack, e.g., between the hours of about 4 am to about 6 am, e.g. at a time normally substantially distant form any previously administered symptomatic asthma therapy.

The effectiveness of the methods described herein for reducing inflammatory conditions, for example in inflammatory airways diseases or disorders, can be demonstrated in a human, an animal model, e.g., a mouse or rat model, of airways inflammation or other inflammatory conditions, for example as described by Szarka et al, J. Immunol. Methods (1997) 202:49-57; Renzi et al, Am. Rev. Respir. Dis. (1993) 148:932-939; Tsuyuki et al., J. Clin. Invest. (1995) 96:2924-2931; and Cernadas et al (1999) Am. J. Respir. Cell Mol. Biol. 20:1-8.

Airway obstruction can be evaluated using spirometry measurements. Spirometry can measure (i) forced vital capacity (FVC), and (ii) forced expiratory volume in 1 second (FEV1). The maximal volume of air forcibly exhaled from the point of maximal inhalation, the FEV1, is the volume of air exhaled during the first second of the FVC. Airflow obstruction can be indicated by reduced FEV1 and FEV1/FVC values relative to reference or predicted values. The severity of obstruction can be assessed by comparing a subject's spirometric measurements with reference values based on age, height, sex, and race (American Thoracic Society, 1991). Obstructed flow of air from the lungs can be indicated by a reduced ratio of FEV1/FVC, i.e., less than 65 percent the lower limit of normal reference value. Reversibility of the obstruction can be indicated by an increase of in FEV 1 after administration of a calpain inhibitor to the subject.

Calpain Inhibitors

In various embodiments, the invention described herein relates to the use of calpain inhibitors to reduce inflammation. Such inhibitors can take a variety of different forms, such as small organic molecules, peptides, small interfering RNA's (siRNAs), proteins (such as calpastatin), and anti-calpain antibodies.

Based on the present findings, inflammatory responses can be initiated or enhanced by the action of endoproteolytic processing of E-cadherin or occludin by calpains. Thus, in one embodiment compounds can reduce inflammatory responses by preventing calpain from generating E-cadherin or occludin cleavage products by interactions with the active site cysteine of calpain 1 or calpain 2 (see for example, Hosfield, C. M., Elce, J. S., Jia, Z. (2004). In another embodiment, compounds can reduce inflammatory responses by preventing activation of calpain by Ca²⁺ (Pal et al., 2003, J Mol Biol. 343(4): 1049-53; Hosfield et al., 1999, Structure (Camb). (12):1521-6; Arthur et al., 1995, EMBO J. 18(24):6880-9)

Calpains are mammalian calcium-dependent neutral cysteine proteases involved in a variety of biological processes. Calpains are referred to as cysteine proteases because they include a cysteine residue that plays an important role in the catalytic process. In the presence of calcium, a cysteine protease catalytic triad forms when three amino acid residues (Cys 105, His262, and Asn 286) are brought together in the active site.

Two major classes of calpains are known, mu-calpain (calpain 1) and m-calpain (calpain 2), which differ in their sensitivities toward calcium. Tissue-specific forms of calpain also have been identified. Substrates of calpain include cellular proteins such as cytoskeletal proteins, membrane-bound receptors, calmodulin binding proteins, myofibrillar proteins, enzymes, and transcription factors.

Without wishing to be bound by theory, different classes of calpains can exhibit different substrate specificities. For example, calpain 1 may exhibit a preference for cleaving occludin whereas calpain 2 may exhibit a preference for cleaving E-cadherin. Alternatively, calpain 1 may exhibit a preference for cleaving E-cadherin whereas calpain 2 may exhibit a preference for cleaving occludin. One skilled in the art will recognize that specificity exhibited by a protease for a substrate need not necessarily indicate a higher affinity for the substrate. Rather, the biological activity of an enzyme can be regulated by parameters other than substrate recognition. For example, post-translational modification, subcellular distribution and the presence or absence of interacting proteins can affect the activity of a protease. In the case of calpain, such regulation can occur, in part due to subcellular redistribution upon the activation of a signaling pathway (for example a PAMP initiated signaling cascade) may cause calpain 1 and calpain 2 to undergo differential redistribution, such that each class of calpain is exposed to a differential population of potential substrates. One skilled in the art will recognize that the inhibition of both calpain 1 and calpain 2 activity maybe required to reduce inflammation in a subject having, or at risk of having, an inflammation related disease or disorder. Class specific inhibitors of calpain activity are also known in the art. For example, Z-Leu-Abu-CONH—CH₂—CH(OH)—C₆H₄-3-OC₆H₄(3-CF₃) is a calpain 1 inhibitor whereas Z-Leu-Abu-CONH—CH₂-2-pyridyl is a calpain 2 inhibitor. See additional examples of isoform specific inhibitors of calpain activity; see Li et al, 1996 J. Med. Chem. 39, 4089-4098.

As described herein, calpain inhibitors can block the transmigration of leukocytes through an epithelium layer and be used to treat or prevent inflammatory conditions. In specific embodiments, the use of one or more inhibitors capable of inhibiting calpain 1 activity can be useful for preventing or inhibiting the re-organization, stabilization or formation of cell junctions at epithelial cell contacts. Thus, in one embodiment, an inhibitor capable of inhibiting calpain 1 activity can be administered to a subject having, or at risk of having an inflammatory condition in the airway. In one embodiment, administration of the one or more inhibitors capable of inhibiting calpain 1 activity to a subject can block dissolution of tight junctions in the subject and inhibit transmigration of leukocytes across the airway epithelium into the airway lumen. In another embodiment, administration of one or more inhibitors capable of inhibiting calpain 1 activity to a subject can block dissolution of adherens junctions in the subject and inhibit transmigration of leukocytes across the airway epithelium into the airway lumen. In still a further embodiment, administration of one or more inhibitors capable of inhibiting calpain 1 activity to a subject will block dissolution of any cell junction acting as a barrier to leukocyte transmigration across the barrier and thus inhibit transmigration of leukocytes across the epithelial layer.

In still further embodiments, the use of one or more inhibitors capable of inhibiting calpain 2 activity can be useful for preventing or inhibiting the re-organization of cell junctions at epithelial cell contacts. Thus, in one embodiment, an inhibitor capable of inhibiting calpain 2 activity can be administered to a subject having, or at risk of having an inflammatory condition in the airway. In one embodiment, administration of the one or more inhibitors capable of inhibiting calpain 2 activity to a subject can block dissolution of tight junctions in the subject and inhibit transmigration of leukocytes across the airway epithelium into the airway lumen. In another embodiment, administration of one or more inhibitors capable of inhibiting calpain 2 activity to a subject can block dissolution of adherens junctions in the subject and inhibit transmigration of leukocytes across the airway epithelium into the airway lumen. In still a further embodiment, administration of one or more inhibitors capable of inhibiting calpain 2 activity to a subject will block dissolution of any cell junction acting as a barrier to leukocyte transmigration across the barrier and, thus inhibit transmigration of leukocytes across the epithelial layer.

In still further embodiments, the use of one or more inhibitors capable of inhibiting calpain 1 and calpain 2 activity can be useful for preventing or inhibiting the re-organization of cell junctions at epithelial cell contacts. Thus, in one embodiment, an inhibitor capable of inhibiting calpain 1 and calpain 2 activity can be administered to a subject having, or at risk of having an inflammatory condition in the airway. In one embodiment, administration of the one or more inhibitors capable of inhibiting calpain 1 and calpain 2 activity to a subject can block dissolution of tight junctions in the subject and inhibit transmigration of leukocytes across the airway epithelium into the airway lumen. In another embodiment, administration of one or more inhibitors capable of inhibiting calpain 1 and calpain 2 activity to a subject can block dissolution of adherens junctions in the subject and inhibit transmigration of leukocytes across the airway epithelium into the airway lumen. In still a further embodiment, administration of one or more inhibitors capable of inhibiting calpain 1 and calpain 2 activity, to a subject will block dissolution of any cell junction acting as a barrier to leukocyte transmigration across the barrier and, thus inhibit transmigration of leukocytes across the epithelial layer.

Inhibition of calpain has provided therapeutic possibilities for a number of different diseases, including cerebral ischemia (e.g., strokes), traumatic brain injury, subarachnoid hemorrhage, chronic neurodegeneration (e.g., Huntington's disease, Parkinson's disease, and amyotrophic lateral sclerosis), Alzheimer's disease, cardiac ischemia (e.g., myocardial infarction), muscular dystrophy, cataracts and thrombotic platelet aggregation, restenosis.

Calpain inhibitors can take on several formulations including dipeptides or larger multimers (see for example: Donkor, I. O., Korukonda, R., Huang, T. L., LeCour, L., Jr. (2003). Peptidyl aldehyde inhibitors of calpain incorporating P2-proline mimetics. Bioorg Med Chem Lett. 13(5):783-4; Inoue J., Nakamura M., Cui, Y. S., Sakai, Y., Sakai, O., Hill, J. R., Wang, K. K., Yuen, P. W. (2003). Structure-activity relationship study and drug profile of N-(4-fluorophenylsulfonyl)-L-valyl-L-leucinal (SJA6017) as a potent calpain inhibitor. J Med Chem. 27;46(5):868-71; and Montero, A., Albericio, F., Royo, M., Herradon, B. (2004). Solid-phase combinatorial synthesis of peptide-biphenyl hybrids as calpain inhibitors. Org Lett. 6(22):4089-92) as well as other organic compounds (see for example: Nakamura, M., Miyashita, H., Yamaguchi, M., Shirasaki, Y., Nakamura, Y., Inoue, J. (2003). Novel 6-hydroxy-3-morpholinones as cornea permeable calpain inhibitors. Bioorg Med Chem. 11(24):5449-60). Calpain activity can also be inhibited by administration of calpain antibodies, a technique that has been previously shown to inhibit other enzymatic processes.

A wide variety of compounds have been demonstrated to have activity in inhibiting the proteolytic action of calpains. Examples of calpain inhibitors that are useful in the practice of the invention include N-acetyl-leucyl-leucylmethional (ALLM or calpain inhibitor II), N-acetyl-leucyl-leucyl-norleucinal (ALLN or calpain inhibitor 1), calpain inhibitor III (carbobenzoxy-valyl-phenylalanal; Z-Val-Phe-CHO), calpain inhibitor IV (Z-LLY-FMK; Z-LLY-CH.sub.2 F where Z=benzyloxycarbonyl), calpain inhibitor V (Mu-Val-HPh-FMK where Mu is morphlinoureidyl and Hph is homophenylalanyl), calpeptin (benzyloxycarbonyldipeptidyl aldehyde; Z-Leu-Nle-CHO), calpain inhibitor peptide (Sigma No. C9181), calpastatin, acetyl-calpastatin (acetyl calpain inhibitor fragment, 184-210), leupeptin, mimetics thereof and combinations there, AK275, MDL28170 and E64. Additional calpain inhibitors are described in the following U.S. patents, incorporated herein by reference, U.S. Pat. Nos. 5,716,980; 5,714,471; 5,693,617; 5,691,368; 5,679,680; 5,663,294, 5,661,150; 5,658,906; 5,654,146; 5,639,783; 5,635,178; 5,629,165; 5,622,981; 5,622,967; 5,621,101; 5,554,767; 5,550,108; 5,541,290; 5,506,243; 5,498,728; 5,498,616; 5,461,146; 5,444,042; 5,424,325; 5,422,359; 5,416,117; 5,395,958; 5,340,922; 5,336,783; 5,328,909; 5,135,916.

In an exemplary embodiment, the invention includes the use of (a) inhibitors that act on or bind to an active site on calpain, for example, MDL 28170 Calpain Inhibitor IV, Calpeptin, SJA6017, N-(4-fluorophenylsulfonyl)-L-valyl-L-leucinal, AK295, Z-Leu-aminobutyric acid-CONH(CH.sub.2).sub.3-morpholine; Z=benzyloxycarbonyl, AK275, Z-Leu-Abu-CONH-CH2CH3; (Abu=.chi.-aminobutyric acid) Z=benzyloxycarbonyl; (b) calpastatin or calpastatin peptide mimetics such as CS 27-mer peptide (Calpain Inhibitor Peptide-amino acid sequence of: D-P-M-S-S-T-Y-I-E-E-L-G-K-R-E-V-T-I-P-P-K-Y-R-E-L-L-A) (SEQ ID NO: 43); (c) compounds that bind to the calpain calcium binding domain such as PD 150606, [3-(4-Iodophenyl)-2-mercapto-(benzyloxycarbonyl)-2-propenoic acid] PD 1151746, 3-(5-fluoro-3-indolyl)-2-mercapto-(benzyloxycarbonyl)-2-propenoic acid; (d) RNAi against calpain small subunit; or (e) expression of exogenous calpastatin by vector transfection or viral infection.

Calpastatin is an endogenous protease inhibitor that acts specifically on calpain (a calcium-dependent cysteine protease). It consists of four repetitive sequences of 120 to 140 amino acid residues (domains I, II, III and IV), and an N-terminal non-homologous sequence (L). The calpastatin promoter contains a single cAMP-responsive element (GTCA) and cAMP signaling can lead to increase in calpastatin gene transcription and reduction in calpain-mediated proteolysis (Cong et al, 1998 Biochimica et Biophysica Acta.1443:186-192.). β-agonists like cimaterol and clenbuterol which generate cAMP upon receptor binding have been shown to upregulate calpastatin expression in bovine skeletal muscle and rat hippocampus cells (Rami et al, (2003), Neuroscience Research. 47:373-382; Parr et al., 1992, Eur. J. Biochem. 208:333-339.). Hypoxia has also been shown to increase calpastatin mRNA is cardiomyocytes Hypoxia in cardiomyocytes increased calpastatin mRNA but not protein levels and therefore did not inhibit calpain proteolytic activity (Lin, Mol Cell Biochem. 2004; 265:63-70). According, in one embodiment, cAMP, β-agonists of hypoxia can be used to upregulate calpastatin expression so as to inhibit calpain activity and reducing inflammation or inhibit leukocyte transmigration across an epithelium in a subject.

A more detailed overview of calpains and calpain inhibitors can be found in Wang, et al., Advances in Pharmacology, 37:117-52 (1997). Patents disclosing calpain inhibitors include: Zimmerman, et al., U.S. Pat. No. 5,374,623; Wang, et al., U.S. Pat. No. 5,760,048; Munoz, et al., U.S. Pat. No. 5,872,101; Munoz, et al., U.S. Pat. No. 5,969,100; and Spruce, et al., U.S. Pat. No. 6,004,933, and the calpain inhibitors disclosed therein are hereby incorporated by reference.

Calpain inhibitors are commercially available. Exemplary protein calpain inhibitors are MDL28170, calpeptin and calpain inhibitor IV. Other suitable calpain inhibitors are listed in the following tables.

TABLE 1
General Calpain Inhibitors
General Calpain Inhibitors

| Product | Company | Catalog # |
|---|---|---|
| Calpastatin, human erythrocytes | Calbiochem | 208901 |
| Calpastatin, human, recombinant | Calbiochem | 208900 |
| Acetyl-Calpastatin, Acetyl Calpain | Sigma | C4285 |
| Inhibitor fragment, 184-210 Ac-D-P- | | |
| M-S-S-T-Y-I-E-E-L-G-K-R-E-V-T-I- | | |
| P-P-K-Y-R-E-L-L-A-NH.sub.2 | | |
| Calpain Inhibitor Peptide D-P-M-S-S- | Sigma | C9181 |
| T-Y-I-E-E-L-G-K-R-E-V-T-I-P-P-K- | | |
| Y-R-E-L-L-A | | |
| Calpain Inhibitor I | Roche | 1086090 |
| N-acetly-L-L-norleucinal | BioMol | P-120 |
| ALLN | Fluka | 21277 |
| ALLN | Sigma | A6185 |
| ALLN | Calbiochem | 208719 |
| Calpain Inhibitor II | Roche | 1086103 |
| N-acetly-L-L-methional | Fluka | 21278 |
| ALLM | Calbiochem | 208721 |
| ALLM | Sigma | A6060 |
| ALLM | BioMol | PI-100 |
| Calpain Inhibitor III carbobenzoxy- | Calbiochem | 208722 |
| valyl-phenylalanal MDL #28170 Z- | | |
| Val-Phe-CHO (Z = benzyloxycarbonyl) | | |
| Calpain Inhibitor IV Z-LLY-FMK Z-L- | Calbiochem | 208724 |
| L-Y-CH.sub.2F (Z = benzyloxycarbonyl) | | |
| Calpain Inhibitor V Mu-Val-HPh-FMK | Calbiochem | 208726 |
| (Mu = morphilinoureidyl) (HPh = | | |
| homophenylalanyl) | | |
| Calpeptin | BioMol | PI-101 |
| benzyloxycarbonyldipeptidyl aldehyde | Calbiochem | 03-34-0051 |
| Z-Leu-Nle-CHO | | |
| (Z = benzyloxycarbonyl) | | |
| trans-Epoxy succinyl-L-leucylamido- | BioMol | PI-105 |
| (4-guanidino) butane | | |
| Z-Leu-Leu-CHO | BioMol | PI-116 |
| MDL-28170 | BioMol | PI-130 |

TABLE 2
Anti-calpain antibodies as calpain inhibitors
Calpain Antibodies

| | | |
|---|---|---|
| Product | Company | Catalog # |

Mu-Calpain, large Subunit

| | | |
|---|---|---|
| Anti-mu-Calpain, 80 kDa subunit, | Affinity Bioreagents | MA3-940 |
| Clone 9A4H8D3, mouse | | |
| Anti-mu-Calpain, 80 kDa subunit, | BioMol | SA-257 |
| Clone 9A4H8D3, mouse | | |
| Anti-mu-Calpain, 80 kDa subunit, | Affinity Bioreagents | MA3-941 |
| Clone 2H2A7C2, mouse | | |
| Anti-mu-Calpain, 80 kDa subunit, | BioMol | SA-256 |
| Clone 2H2A7C2, mouse | | |
| Anti-mu-Calpain, 80 kDa subunit, | Research Diagnostics, | RDI-UCALPAINabm |
| PC-6, mouse | Inc. | |
| Anti-mu-Calpain, 80 kDa subunit, | Research Diagnostics, | RDI-CALPN1CabG |
| goat | Inc. | |
| Anti-mu-Calpain, 80 kDa subunit, | Research Diagnostics, | RDI-CALPN1NabG |
| goat | Inc. | |
| Anti-mu-Calpain, 80 kDa subunit, | Triple Point Biologics | RP1CALPAIN1 |
| rabbit domain I | | |
| Anti-mu-Calpain, 80 kDa subunit, | Triple Point Biologics | RP2CALPAIN1 |
| rabbit domain I | | |
| Anti-mu-Calpain, 80 kDa subunit, | Triple Point Biologics | RP3CALPAIN1 |
| rabbit domain IV | | |
| Anti-mu-Calpain, 80 kDa subunit, | Triple Point Biologics | RP4CALPAIN1 |
| rabbit domain IV | | |

M-Calpain, large subunit

| | | |
|---|---|---|
| Anti-m-Calpain, 80 kDa subunit, | Affinity Bioreagents | MA3-942 |
| Clone 107-82, mouse | | |
| Anti-m-Calpain, 80 kDa subunit, | BioMol | SA-255 |
| Clone 107-82, mouse | | |
| Anti-m-Calpain, 80 kDa subunit, PC1, | Research Diagnostics, | RDI-MCALPAINNabr |
| rabbit | Inc. | |
| Anti-m-Calpain, 80 kDa subunit, PC1, | Research Diagnostics, | RDI-CALPN2NabG |
| goat | Inc. | |
| Anti-m-Calpain, 80 kDa subunit, PC1, | Triple Point Biologics | RP1CALPAIN2 |
| rabbit, domain III | | |
| Anti-m-Calpain, 80 kDa subunit, PC1, | Triple Point Biologics | RP2CALPAIN2 |
| rabbit, domain I | | |
| Anti-m-Calpain, 80 kDa subunit, PC1, | Triple Point Biologics | RP3CALPAIN2 |
| rabbit, domain IV | | |
| Anti-m-Calpain, 80 kDa subunit, PC1, | Triple Point Biologics | RP4CALPAIN2 |
| rabbit, domain III | | |

Calpain, small subunit

| | | |
|---|---|---|
| Anti-Calpain, 28 kDa subunit, Clone | Affinity Bioreagents | MA3-943 |
| 156, mouse | | |
| Anti-Calpain, 28 kDa subunit, goat | Research Diagnostics, | RDI-CALPRGCabG |
| | Inc. | |
| Anti-Calpain, 28 kDa subunit, goat | Research Diagnostics, | RDI-CALPRGIabG |
| | Inc. | |

Calpain 3 (p94)

| | | |
|---|---|---|
| Anti-Calpain 3, rabbit, Insert I | Triple Point Biologics | RP1CALPAIN3 |
| Anti-Calpain 3, rabbit, Insert II | Triple Point Biologics | RP2CALPAIN3 |
| Anti-Calpain 3, rabbit, domain II | Triple Point Biologics | RP3CALPAIN3 |
| Anti-Calpain 3, rabbit, domain I | Triple Point Biologics | RP4CALPAIN3 |

Calpain 3 (Lp82/85)

| | | |
|---|---|---|
| Anti-Lp85, rabbit, domain IV | Triple Point Biologics | RP1P85CALPAIN |
| Anti-Lp82/85, rabbit, domain III | Triple Point Biologics | RP1LP82/85CALPAIN |

TABLE 3
Calpastatin calpain inhibitor
Calpastatin

| | | |
|---|---|---|
| Anti-Calpastatin, Clone 1F7E3D10, | Affinity Bioreagents | MA3-944 |
| mouse | | |
| Anti-Calpastatin, Clone 1F7E3D10, | BioMol | SA-284 |
| mouse | | |
| Anti-Calpastatin, Clone 2G11D6, | Affinity Bioreagents | MA3-945 |
| mouse | | |
| Anti-Calpastatin, Clone 2G11D6, | BioMol | SA-283 |
| mouse | | |

RNA interference (RNAi) is a process whereby double-stranded RNA (dsRNA) induces the sequence-specific degradation of homologous mRNA in animals and plant cells (Hutvagner and Zamore, 2002, Curr. Opin. Genet. Dev. 12:225-232; Sharp, 2001, Genes Dev. 15:485-490). In mammalian cells, RNAi can be triggered by, e.g., approximately 21-nucleotide (nt) duplexes of small interfering RNA (siRNA) (Chiu et al., 2002, Mol. Cell. 10:549-561; Elbashir et al., 2001, Nature 411:494-498), or by micro-RNAs (miRNA), functional small-hairpin RNA (shRNA), or other dsRNAs which are expressed in vivo using DNA templates with RNA polymerase III promoters (Zeng et al., 2002, Mol. Cell 9:1327-1333; Paddison et al., 2002, Genes Dev., 16:948-958; Lee et al., 2002, Nature Biotechnol. 20:500-505; Paul et al., 2002, Nature Biotechnol. 20:505-508; Tuschl, 2002, Nature Biotechnol. 20:440-448; Yu et al., 2002, Proc. Natl. Acad. Sci. USA, 99:6047-6052; McManus et al., 2002, RNA 8:842-850; Sui et al., 2002, Proc. Natl. Acad. Sci. USA 99:5515-5520).

Examples of molecules that can be used to decrease expression of an inhibitory molecule comprise double-stranded RNA (dsRNA) molecules that can function as siRNAs targeting nucleic acids encoding the inhibitory molecule and that comprise 16-30, e.g., 16, 17, 18, 19, 20, 21, 22, 23, 24, 25, 26, 27, 28, 29, or 30 nucleotides in each strand, wherein one of the strands is substantially complementary to, e.g., at least 80% (or more, e.g., 85%, 90%, 95%, or 100%) complementary to, e.g., having 3, 2, 1, or 0 mismatched nucleotide(s), a target region, e.g., a transcribed region of a nucleic acid and the other strand is identical or substantially identical to the first strand. The dsRNA molecules can be chemically synthesized, or can be transcribed in vitro from a DNA template, or in vivo from an engineered RNA precursor, e.g., shRNA. The dsRNA molecules may be designed using methods known in the art (e.g., “The siRNA User Guide,” available at www rockefeller.edu/labheads/tuschl/siRNA) and can be obtained from commercial sources, e.g., Dharmacon, Inc. (Lafayette, Colo.) and Ambion, Inc. (Austin, Tex.).

In one embodiment, the invention includes the use of one or more siRNA nucleic acids to inhibit calpain activity. Exemplary siRNA sequences suitable for use with the methods and compositions described herein include sequences that comprise SEQ ID NOs: 27-42 and sequences that are at least 95, 96, 97, 98, or 99% identical to SEQ ID NOs: 27-42.

Based on the present findings, inflammatory responses can be initiated or enhanced by the action of endoproteolytic processing of E-cadherin or occludin by calpains. Thus, in one embodiment siRNA nucleic acids capable of inhibiting calpain activity can reduce inflammatory responses by reducing expression of a calpain protein and preventing calpain from generating E-cadherin or occludin cleavage products.

Negative control siRNAs generally have the same nucleotide composition as the selected siRNA, but without significant sequence complementarity to the targeted genome. Such negative controls can be designed by randomly scrambling the nucleotide sequence of the selected siRNA; a homology search can be performed to ensure that the negative control lacks homology to any other gene in the appropriate genome. In addition, negative control siRNAs can be designed by introducing one or more base mismatches into the sequence.

The siRNAs for use as described herein can be delivered to a cell by methods known in the art and as described herein in using methods such as transfection utilizing commercially available kits and reagents. Viral infection, e.g., using a lentivirus vector can be used.

An siRNA or other oligonucleotide can also be introduced into the cell by transfection with an heterologous target gene using carrier compositions such as liposomes, which are known in the art, e.g., Lipofectamine™ 2000 (Invitrogen, Carlsbad, Calif.) as described by the manufacturer for adherent cell lines. Transfection of dsRNA oligonucleotides for targeting endogenous genes can be carried out using Oligofectamine™ (Invitrogen, Carlsbad, Calif.). The effectiveness of the oligonucleotide can be assessed by any of a number of assays following introduction of the oligonucleotide into a cell. These assays comprise, but are not limited to, Western blot analysis using antibodies that recognize the targeted gene product following sufficient time for turnover of the endogenous pool after new protein synthesis is repressed, and Northern blot analysis to determine the level of existing target mRNA.

Still further compositions, methods and applications of RNAi technology for use as described herein are provided in U.S. Pat. Nos. 6,278,039, 5,723,750 and 5,244,805, which are incorporated herein by reference.

Another aspect of the invention provides aerosols for the delivery of siRNA nucleic acids capable of inhibiting calpain activity to the respiratory tract. The respiratory tract includes the upper airways, including the oropharynx and larynx, followed by the lower airways, which include the trachea followed by bifurcations into the bronchi and bronchioli. The upper and lower airways are called the conductive airways. The terminal bronchioli then divide into respiratory bronchioli which then lead to the ultimate respiratory zone, the alveoli, or deep lung.

Administration by inhalation may be oral and/or nasal. Examples of pharmaceutical devices for aerosol delivery include metered dose inhalers (MDIs), dry powder inhalers (DPIs), and air jet nebulizers. Exemplary nucleic acid delivery systems by inhalation which can be readily adapted for delivery of siRNA nucleic acids capable of inhibiting calpain activity are described in, for example, U.S. Pat. Nos. 5,756,353; 5,858,784; and PCT applications WO98/31346; WO98/10796; WO00/27359; WO01/54664; WO02/060412. Other aerosol formulations that may be used for delivering the double-stranded RNAs are described in U.S. Pat. Nos. 6,294,153; 6,344,194; 6,071,497, and PCT applications WO02/066078; WO02/053190; WO01/60420; WO00/66206. Further, methods for delivering RNAi constructs can be adapted from those used in delivering other oligonucleotides (e.g., an antisense oligonucleotide) by inhalation, such as described in Templin et al., Antisense Nucleic Acid Drug Dev, 2000, 10:359-68; Sandrasagra et al., Expert Opin Biol Ther, 2001, 1:979-83; Sandrasagra et al., Antisense Nucleic Acid Drug Dev, 2002, 12:177-81.

In other embodiments, the subject siRNA nucleic acids capable of inhibiting calpain activity can be provided in liposomes or supramolecular complexes (such as described above) appropriately formulated for pulmonary delivery.

The one aspect, the calpain inhibitors described herein can include polypeptides comprising the N-terminal cytoplasmic domain of occludin can be used in conjunction with the methods described herein. As described herein, cleavage of occludin in the N-terminal cytoplasmic domain by calpain causes a reduction in cell junction integrity, formation or stability. Accordingly, the presence of a polypeptide having a sequence identical to that of the N-terminal cytoplasmic domain of occludin can be used to inhibit the ability of calpain. Without wishing to be bound by theory, expression of an N-terminal cytoplasmic domain of occludin in a cell can titrate the cellular pool of endogenously expressed calpain and reduce or inhibit the rate of calpain mediated cleavage of endogenous occludin, endogenous E-cadherin or endogenous Ezrin. In one embodiment, expression of an N-terminal cytoplasmic domain of occludin will result in the complete inhibition of cellular calpain activity. In another embodiment, expression of an N-terminal cytoplasmic domain of occludin will result in the partial inhibition of cellular calpain activity. Such partial inhibition of cellular calpain activity in cell comprised within an epithelial layer can inhibit leukocyte transmigration through the epithelial layer. In one embodiment, the inhibition of leukocyte transmigration through the epithelial layer by expressing an N-terminal cytoplasmic domain of occludin in cells comprised in an epithelial layer of a subject having or at risk of having an inflammation related disease or disorder can be useful for reducing or inhibiting inflammation related disease or disorder or symptoms associated with the inflammation related disease or disorder.

The calpain cleavage site in the N-terminal cytoplasmic domain of occludin is within the first transmembrane domain of occludin. Thus, in one embodiment, calpain inhibitors, as described herein can comprise amino acids from about 1 to about 66 of occludin. In another embodiment, a calpain inhibitor, as described herein, can comprise the amino acid sequence MSSRPLESPPPYRPDEFKPNHYAPSNDIYGGEMHVRPMLSQPAYSFYPEDEILHFYKW TSPPGVIR (SEQ IN NO: 44). In another embodiment, the calpain inhibitor, as described herein, can consist essentially of the amino acid sequence shown in SEQ ID NO: 44. The calpain inhibitors of the invention can have comprise a sequence of to any region within the sequence shown in SEQ ID NO: 44.

E-cadherin is a single pass transmembrane domain having a cytoplasmic C-terminal domain consisting of amino acids 734 to 879 in the sequence of human E-cadherin. Because calpain cleaves intracellular sequences, the E-cadherin calpain cleavage site described herein is located within this intracellular domain. Accordingly, in one embodiment, calpain inhibitors, as described herein can comprise amino acids from about 734 to 879 in the sequence of human E-cadherin. In another embodiment, a calpain inhibitor, as described herein, can comprise the amino acid sequence RAVVKEPLLPPEDDTRDNVYYYDEEGGGEEDQDFDLSQLHRGLDARPEVTRNDVAP TLMSVPRYLPRPANPDEIGNFIDENLKAADTDPTAPPYDSLLVFDYEGSGSEAASLSS LNSSESDKDQDYDYLNEWGNRFKKLADMYGGGEDD (SEQ IN NO: 45). In another embodiment, the calpain inhibitor, as described herein, can consist essentially of the amino acid sequence shown in SEQ ID NO: 45. The calpain inhibitors of the invention can have comprise a sequence of to any region within the sequence shown in SEQ ID NO: 45.

The calpain inhibitors shown in SEQ ID NO: 44 and 45 can be used alone, or in conjunction with other calpain inhibitors known in the art. For example, one skilled in the art will recognize that the calpain inhibitors shown in SEQ ID NO: 44 or 45 can be used in conjunction with active site directed calpain inhibitors including, but not limited to, Calpain Inhibitor Peptide, Calpain Inhibitor I, N-acetly-L-L-norleucinal, ALLN, Calpain Inhibitor II, N-acetly-L-L-methional, ALLM, Calpain Inhibitor III, Calpain Inhibitor IV, Calpain Inhibitor V, Calpeptin. benzyloxycarbonyldipeptidyl aldehyde, trans-Epoxy succinyl-L-leucylamido-(4-guanidino) butane, and Z-Leu-Leu-CHO, E64D, SJA6017, N-(4-fluorophenylsulfonyl)-L-valyl-L-leucinal, AK295, benzyloxycarbony-Leu-aminobutyric acid-CONH(CH2)3-morpholine, AK275, and benzyloxycarbony-Leu-Abu-CONH—CH2CH3, and derivatives thereof or calpain inhibitors that bind to a calpain calcium binding domain, including but not limited to PD 150606, [3-(4-Iodophenyl)-2-mercapto-(benzyloxycarbonyl)-2-propenoic acid], and PD 1151746, 3-(5-fluoro-3-indolyl)-2-mercapto-(benzyloxycarbonyl)-2-propenoic acid, or any derivatives thereof. Also suitable for use with the calpain inhibitors shown in SEQ ID NO: 44 or 45 are calpain inhibitors comprising calpastatin or a calpastatin peptide mimetic.

One skilled in the art will recognize that the calpain inhibitors shown in SEQ ID NO: 44 and 45 can be used in conjunction with a small molecule, a protein, a peptide, a peptidomimetic, small interfering RNA, a short hairpin RNA, a microRNA, and an anti-calpain antibody, and derivative thereof. In one aspect, the calpain inhibitor acts or binds to an active site of calpain. For example, a method of reducing inflammation in a subject in need thereof can comprise administration of a calpain inhibitor comprising the sequence shown in SEQ ID NO: 44 or 45 and an siRNA having the sequence shown in any of SEQ ID NO: 27-42. Accordingly, other calpain inhibitors

In one embodiment, the calpain inhibitor described herein can comprise, or consist essentially of amino acids from about position 1 to about 10 of SEQ ID NO: 44 or 45. In another embodiment, the calpain inhibitor described herein can comprise, or consist essentially of amino acids from about position 5 to about 15 of SEQ ID NO: 44 or 45. In another embodiment, the calpain inhibitor described herein can comprise, or consist essentially of amino acids from about position 10 to about 20 of SEQ ID NO: 44 or 45. In another embodiment, the calpain inhibitor described herein can comprise, or consist essentially of amino acids from about position 15 to about 25 of SEQ ID NO: 44 or 45. In another embodiment, the calpain inhibitor described herein can comprise, or consist essentially of amino acids from about position 20 to about 30 of SEQ ID NO: 44 or 45. In another embodiment, the calpain inhibitor described herein can comprise, or consist essentially of amino acids from about position 25 to about 35 of SEQ ID NO: 44 or 45. In another embodiment, the calpain inhibitor described herein can comprise, or consist essentially of amino acids from about position 30 to about 40 of SEQ ID NO: 44 or 45. In another embodiment, the calpain inhibitor described herein can comprise, or consist essentially of amino acids from about position 35 to about 45 of SEQ ID NO: 44 or 45. In another embodiment, the calpain inhibitor described herein can comprise, or consist essentially of amino acids from about position 40 to about 50 of SEQ ID NO: 44 or 45. In another embodiment, the calpain inhibitor described herein can comprise, or consist essentially of amino acids from about position 45 to about 55 of SEQ ID NO: 44 or 45. In another embodiment, the calpain inhibitor described herein can comprise, or consist essentially of amino acids from about position 50 to about 60 of SEQ ID NO: 44 or 45. In another embodiment, the calpain inhibitor described herein can comprise, or consist essentially of amino acids from about position 55 to about 66 of SEQ ID NO: 44 or 45.

In another embodiment, the calpain inhibitor described herein can comprise, or consist essentially of amino acids from about position 60 to about 70 of SEQ ID NO: 45. In another embodiment, the calpain inhibitor described herein can comprise, or consist essentially of amino acids from about position 65 to about 75 of SEQ ID NO: 45. In another embodiment, the calpain inhibitor described herein can comprise, or consist essentially of amino acids from about position 70 to about 80 of SEQ ID NO: 45. In another embodiment, the calpain inhibitor described herein can comprise, or consist essentially of amino acids from about position 75 to about 85 of SEQ ID NO: 45. In another embodiment, the calpain inhibitor described herein can comprise, or consist essentially of amino acids from about position 90 to about 100 of SEQ ID NO: 45. In another embodiment, the calpain inhibitor described herein can comprise, or consist essentially of amino acids from about position 95 to about 105 of SEQ ID NO: 45. In another embodiment, the calpain inhibitor described herein can comprise, or consist essentially of amino acids from about position 100 to about 110 of SEQ ID NO: 45. In another embodiment, the calpain inhibitor described herein can comprise, or consist essentially of amino acids from about position 105 to about 115 of SEQ ID NO: 45. In another embodiment, the calpain inhibitor described herein can comprise, or consist essentially of amino acids from about position 110 to about 120 of SEQ ID NO: 45. In another embodiment, the calpain inhibitor described herein can comprise, or consist essentially of amino acids from about position 115 to about 125 of SEQ ID NO: 45. In another embodiment, the calpain inhibitor described herein can comprise, or consist essentially of amino acids from about position 120 to about 130 of SEQ ID NO: 45. In another embodiment, the calpain inhibitor described herein can comprise, or consist essentially of amino acids from about position 125 to about 135 of SEQ ID NO: 45. In another embodiment, the calpain inhibitor described herein can comprise, or consist essentially of amino acids from about position 130 to about 140 of SEQ ID NO: 45. In another embodiment, the calpain inhibitor described herein can comprise, or consist essentially of amino acids from about position 135 to about 145 of SEQ ID NO: 45.

In one embodiment, the calpain inhibitors described herein can inhibit or reduce the activation of a calpain. In another embodiment, the calpain inhibitors described herein can inhibit or reduce the association of a calpain with occludin, E-cadherin or ezrin. In another embodiment, the calpain inhibitors described herein can inhibit the enzymatic activity of calpain.

Calpain inhibitors suitable for use with the methods described herein can include variants of the polypeptide shown in SEQ ID NO: 44 or 45. Such variants, can include, but are not limited to polypeptides having one or more conserved amino acid substitutions, wherein an amino acid in the sequence shown on SEQ ID NO: 44 or 45 is either retained or replaced with an amino acid residue of the same type. A conservative amino acid substitution occurs when one amino acid residue is replaced with another that has a similar side chain.

Conservative amino acid substitutions can be made at one or more non-essential amino acid residues. A conservative amino acid substitution can be a substitution in which an amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine), aromatic side chains (e.g., tyrosine, phenylalanine, tryptophan, histidine), aliphatic side chains (e.g., glycine, alanine, valine, leucine, isoleucine), and sulfur-containing side chains (methionine, cysteine). Substitutions can also be made between acidic amino acids and their respective amides (e.g., asparagine and aspartic acid, or glutamine and glutamic acid).

Conservative amino acid substitutions can be utilized in making variants of the calpain inhibitors described herein. For example, replacement of a leucine with an isoleucine or valine, an aspartate with a glutamate, a threonine with a serine, or a similar replacement of an amino acid with a structurally related amino acid, may not have a major effect on the properties of the resulting polypeptide or fusion polypeptide. Whether an amino acid change results in a functional polypeptide or fusion polypeptide can readily be determined by assaying the specific activity of the polypeptide or fusion polypeptide.

Nucleic Acids

One aspect of this invention pertains to polypeptides that can inhibit calpain activity. The polypeptides of this invention can be produced by recombinant DNA techniques. Alternative to recombinant expression, a polypeptide of this invention can be synthesized chemically using standard polypeptide synthesis techniques.

The calpain inhibitors described herein can be prepared using standard peptide synthesis methods, which are well known in the art (see for example Stewart et al., Solid Phase Peptide Synthesis, Pierce Biotechnology, Inc., Rockford, Ill., 1984; Bodanszky, Principles of Peptide Synthesis, Springer-Verlag, New York, 1984; and Pennington et al., Peptide Synthesis Protocols, Humana Press, Totowa, N.J., 1994). Additionally, many companies offer custom peptide synthesis services.

Alternatively, the calpain inhibitors can be prepared using recombinant DNA and molecular cloning techniques. Genes encoding the candidate binding peptides may be produced in heterologous host cells, particularly in the cells of microbial hosts. Preferred heterologous host cells for expression of candidate binding peptides of the present invention are microbial hosts that can be found broadly within the fungal or bacterial families and which grow over a wide range of temperature, pH values, and solvent tolerances. Because transcription, translation, and the protein biosynthetic apparatus are the same irrespective of the cellular feedstock, functional genes are expressed irrespective of carbon feedstock used to generate cellular biomass. Examples of host strains include, but are not limited to, fungal or yeast species such as Aspergillus, Trichoderma, Saccharomyces, Pichia, Candida, Hansenula, or bacterial species such as Salmonella, Bacillus, Acinetobacter, Rhodococcus, Streptomyces, Escherichia, Pseudomonas, Methylomonas, Methylobacter, Alcaligenes, Synechocystis, Anabaena, Thiobacillus, Methanobacterium and Klebsiella.

A variety of expression systems can be used to produce the calpain inhibitors of the present invention. Such vectors include, but are not limited to, chromosomal, episomal and virus-derived vectors, e.g., vectors derived from bacterial plasmids, from bacteriophage, from transposons, from insertion elements, from yeast episomes, from viruses such as baculoviruses, retroviruses and vectors derived from combinations thereof such as those derived from plasmid and bacteriophage genetic elements, such as cosmids and phagemids. The expression system constructs may contain regulatory regions that regulate as well as engender expression. In general, any system or vector suitable to maintain, propagate or express polynucleotide or polypeptide in a host cell may be used for expression in this regard. Microbial expression systems and expression vectors contain regulatory sequences that direct high level expression of foreign proteins relative to the growth of the host cell. Regulatory sequences are well known to those skilled in the art and examples include, but are not limited to, those which cause the expression of a gene to be turned on or off in response to a chemical or physical stimulus, including the presence of regulatory elements in the vector, for example, enhancer sequences. Any of these could be used to construct chimeric genes for production of the any of the binding peptides of the present invention. These chimeric genes could then be introduced into appropriate microorganisms via transformation to provide high level expression of the peptides.

The amino acid sequences of the polypeptides of this invention will enable those of skill in the art to produce polypeptides corresponding to polypeptide sequences of this invention and sequence variants thereof. Such polypeptides can be produced in prokaryotic or eukaryotic host cells by expression of nucleic acids encoding a polypeptide of this invention. The production of these polypeptides can also be done as part of a larger polypeptide. Any of the polypeptides of the invention described herein can be synthesized by chemical methods. Methods for expression of heterologous proteins in recombinant hosts, chemical synthesis of polypeptides, and in vitro translation are well known in the art and are described further in Sambrook J et al.; Berger and Kimmel, Methods in Enzymology, Volume 152, Guide to Molecular Cloning Techniques (1987), Academic Press, Inc., San Diego, Calif.; Gutte B and Merrifield R B, J. Am. Chem. Soc. 91:501-02 (1969); Chaiken I M, CRC Crit. Rev. Biochem. 11:255-301 (1981); Kaiser E T et al., Science 243:187-92 (1989); Merrifield B, Science 232:341-47 (1986); Kent S B H, Ann. Rev. Biochem. 57:957-89 (1988); Offord, R. E. (1980) Semisynthetic Proteins, Wiley Publishing.

Polypeptides can be produced by direct chemical synthesis. Polypeptides can be produced as modified polypeptides, with nonpeptide moieties attached by covalent linkage to the N-terminus and/or C-terminus. In certain embodiments, either the carboxy-terminus or the amino-terminus, or both, are chemically modified. The most common modifications of the terminal amino and carboxyl groups are acetylation and amidation, respectively. Amino-terminal modifications such as acylation (e.g., acetylation) or alkylation (e.g., methylation) and carboxy-terminal-modifications such as amidation, as well as other terminal modifications, including cyclization, can be incorporated into various embodiments. Certain amino-terminal and/or carboxy-terminal modifications and/or polypeptide extensions to the core sequence can provide advantageous physical, chemical, biochemical, and pharmacological properties, such as: enhanced stability, increased potency and/or efficacy, resistance to serum proteases, desirable pharmacokinetic properties, and others. Polypeptides can be used therapeutically to treat disease.

The polypeptide or fusion proteins of this invention can be synthesized in vitro, e.g., by the solid phase polypeptide synthetic method or by recombinant DNA approaches described herein. The solid phase polypeptide synthetic method is an established and widely used method. These polypeptides can be further purified by fractionation on immunoaffinity or ion-exchange columns; ethanol precipitation; reverse phase HPLC; chromatography on silica or on an anion-exchange resin such as DEAE; chromatofocusing; SDS-PAGE; ammonium sulfate precipitation; gel filtration using, for example, Sephadex G-75; or ligand affinity chromatography.

Chimeric or fusion proteins are also provided by this invention. For example, the fusion protein can be a GST member fusion protein in which one or more polypeptides of this invention are fused to the C-terminus of the GST sequences. Alternatively, a fusion protein of this invention can be an HA fusion protein in which a nucleotide sequence of this invention is inserted in a vector, such as pCEP4-HA vector (Herrscher R F et al., Genes Dev. 9:3067-82 (1995)) such that the sequences are fused in frame to an influenza hemagglutinin epitope tag with a polypeptide of this invention. Such fusion proteins can facilitate the purification of a recombinant polypeptides capable of inhibiting calpain activity. Fusion proteins are not limited to polypeptides capable of inhibiting calpain activity; the fusion proteins of the invention can be any protein fused to a polypeptide of this invention.

Fusion proteins and polypeptides produced by recombinant techniques can be secreted and isolated from a mixture of cells and medium containing the protein or polypeptide. Alternatively, the protein or polypeptide can be retained cytoplasmically and the cells harvested, lysed, and the protein isolated. A cell culture can include host cells, media, and other byproducts. Suitable media for cell culture are well known in the art. Protein and polypeptides can be isolated from cell culture media, host cells, or both using techniques known in the art for purifying proteins and polypeptides. Techniques for transfecting host cells and purifying proteins and polypeptides are known in the art.

In view of the foregoing, the nucleotide sequence of a DNA or RNA molecule coding for a nucleic acid of this invention (or a portion thereof) can be used to derive a calpain inhibitor of the invention using the genetic code to translate the DNA or RNA molecule into an amino acid sequence. Thus, description and/or disclosure herein of a nucleic acid sequence of this invention also includes the description and/or disclosure of the amino acid sequence encoded by the nucleic acid sequence. Similarly, description and/or disclosure of a amino acid sequence of this invention herein also includes the description and/or disclosure of all possible nucleic acid sequences that can encode the amino acid sequence.

Nucleic acids disclosed herein can be isolated using standard molecular biology techniques and the sequence information provided herein. For example, using all or portion of a nucleic acid sequence as a hybridization probe, nucleic acids can be isolated using standard hybridization and cloning techniques (e.g., as described in Sambrook, J., Fritsh, E. F., and Maniatis, T. Molecular Cloning: A Laboratory Manual, 2nd ed., Cold Spring Harbor Laboratory, Cold Spring Harbor Laboratory Press, Cold Spring Harbor, N.Y., 1989).

A nucleic acid can be amplified using cDNA, mRNA or genomic DNA as a template and appropriate oligonucleotide primers according to standard PCR amplification techniques. The nucleic acid so amplified can be cloned into an appropriate vector and characterized by DNA sequence analysis. Furthermore, oligonucleotides can be prepared by standard synthetic techniques, e.g., using an automated DNA synthesizer.

In one aspect, the invention provides for nucleic acids encoding a polypeptide capable of inhibiting calpain activity. In one embodiment, the nucleic acid can be a nucleic acid comprising a sequence consisting of, or consisting essentially of a nucleotide sequence encoding a polypeptide that is functionally equivalent a polypeptide having the sequence shown in SEQ ID NO: 44 or 45. Functional equivalency can include identical, reduced or increased ability to inhibit calpain activity. In another embodiment, the nucleic acid can be a nucleotide sequence encoding a polypeptide at least about 60%, 70%, 80%, 85%, 90%, 95%, 98%, 99% homologous or identical with an amino acid sequence of SEQ ID NO: 44 or 45. Percent identity or percent similarity of a DNA or peptide sequence can be determined, for example, by comparing sequence information using the GAP computer program, available from the University of Wisconsin Genetics Computer Group (now part of Accelrys Inc, San Diego, Calif., United States of America). The GAP program utilizes the alignment method of Needleman et al., 1970, as revised by Smith et al., 1981. Briefly, the GAP program defines similarity as the number of aligned symbols (i.e., nucleotides or amino acids) that are similar, divided by the total number of symbols in the shorter of the two sequences. The preferred parameters for the GAP program are the default parameters, which do not impose a penalty for end gaps. See e.g., Schwartz et al., 1979; Gribskov et al., 1986. Nucleic acids that differ due to degeneracy of the genetic code, and still encode the calpain inhibitors described herein are encompassed by the present disclosure.

Co-Treatments

The methods described herein are also useful as co-therapeutic agents for use in combination with other drug substances such as anti-bacterial, anti-viral, anti-inflammatory, bronchodilatory, antihistamine or anti-tussive drug substances, and in the treatment of inflammation-related diseases and disorders such as those mentioned herein, for example as potentiators of therapeutic activity of such drugs or as a means of reducing dosaging or potential side effects of such drugs. A calpain inhibitor can be mixed with the other drug substance in a fixed pharmaceutical composition or it can be administered separately, before, simultaneously with or after the other drug substance.

Exemplary anti-bacterial substances include beta-lactam antibiotics; including penicillins, penicillin G-like drugs (penicillin G, penicillin V, procaine penicillin, benzathine penicillin), Penicillinase-resistant penicillins, Cloxacillin, Dicloxacillin, Methicillin, Nafcillin, Oxacillin, Ampicillin-like drugs; including ampicillin, ampicillin plus sulbactam, amoxicillin, amoxicillin plus clavulanate, Bacampicillin, Broad-spectrum (antipseudomonal) penicillins, Azlocillin, Carbenicillin, Mezlocillin, Piperacillin, Piperacillin plus tazobactam, Ticarcillin, Ticarcillin plus clavulanate, Cephalosporins, Imipenem, meropenem, Aztreonam, Clavulanic acid, sulbactam, and tazobactam, Aminoglycosides, Amikacin, Gentamicin, Kanamycin, Neomycin, Netilmicin, Streptomycin, Tobramycin, Macrolides, Lincomycin, Clindamycin (azithromycin, clarithromycin, clindamycin), Erythromycin, Lincomycin, Tetracyclines, Demeclocycline, Doxycycline, Minocycline, Oxytetracycline, Tetracycline, Chloroamphenicol, Vancomycin, Quinupristin/Dalfopristin, Metronidazole, Rifampin, Spectinomycin, Nitrofurantoin, Quinolones, Cinoxacin, Nalidixic acid, Fluoroquinolones, Ciprofloxacin, Enoxacin, Grepafloxacin, Levofloxacin, Lomefloxacin, Norfloxacin, Ofloxacin, Sparfloxacin, Trovafloxacin, Bacitracin, Colistin, Polymyxin B, and Sulfonamides

Exemplary anti-viral substances include Idoxuridine (IDU), Vidarabine, (adenine arabinoside, ara-A), Trifluridine (triflurothymidine), Acyclovir, Famciclovir, Penciclovir, Ralacyclovir, Ganciclovir, Foscarnet, Ribavirin, Amantadine, Rimantadine, Cidoforvir, Antisense Oligonucleotides, immune globulins, Zidovudine (ZDV, AZT), Didanosine (ddI), Zalcitrabine (ddC), Stavudine (d4T), Lamivudine (3TC), Reverse transcriptase inhibitors (nevirapine, delavirdine), and viral protease inhibitors.

Exemplary anti-inflammatory drugs include steroids, (e.g., gluco-corticosteroids such as budesonide, beclamethasone, fluticasone, ciclesonide or mometasone, or steroids described in WO 02/88167, WO 02/12266, WO 02/100879, WO 04/039827 or WO 02/00679, especially those of Examples 3, 11, 14, 17, 19, 26, 34, 37, 39, 51, 60, 67, 72, 73, 90, 99 and 101); LTB4 antagonists such as those described in U.S. Pat. No. 5,451,700, also LY293111, CGS025019C, CP-195543, SC-53228, BIIL 284, ONO 4057 and SB 209247; LTD4 antagonists such as montelukast and zafirlukast; Dopamine receptor agonists such as cabergoline, bromocriptine, ropinirole and 4-hydroxy-7-[2-[[2-[[3-(2-phenylethoxy)propyl]sulfonyl]ethyl]-amino]e-thyl]-2(3H)-benzothiazolone and pharmaceutically acceptable salts thereof (the hydrochloride being Viozan-AstraZeneca); PDE4 inhibitors such as cilomilast (Ariflo GSK), Roflumilast (Byk Gulden), V-11294A (Napp), BAY19-8004 (Bayer), SCH-351591 (Schering-Plough), Arofylline (Almirall Prodesfarma), PD189659/PD168787 (Parke-Davis), AWD-12-281 (Asta Medica), CDC-801 (Celgene), Se1CID.™. CC-10004 (Celgene), VM554/UM565 (Vernalis), T-440 (Tanabe), KW-4490 (Kyowa Hakko Kogyo), WO 92/19594, WO 93/19749, WO 93/19750, WO 93/19751, WO 99/16766, WO 01/13953, WO 03/104204, WO 03/104205, WO 04/000814, WO 04/000839 and WO 04/005258, WO 04018450, WO 04/018451, WO 04/018457, WO 04/018465, WO 04/018431, WO 04/018449, WO 04/018450, WO 04/018451, WO 04/018457, WO 04/018465, WO 04/019944, WO 04/019945 and WO 04/045607, WO 04/037805 as well as those described in WO 98/18796 and WO 03/39544; A2a agonists such as those described in EP 409595A2, EP 1052264, EP 1241176, WO 94/17090, WO 96/02543, WO 96/02553, WO 98/28319, WO 99/24449, WO 99/24450, WO 99/24451, WO 99/38877, WO 99/41267, WO 99/67263, WO 99/67264, WO 99/67265, WO 99/67266, WO 00/23457, WO 00/77018, WO 00/78774, WO 01/23399, WO 01/27130, WO 01/27131, WO 01/60835, WO 01/94368, WO 02/00676, WO 02/22630, WO 02/96462, WO 03/086408, WO 04/039762, WO 04/039766, WO 04/045618, WO 04/046083; and A2b antagonists such as those described in WO 02/42298.

Exemplary bronchodilatory drugs include anticholinergic or antimuscarinic agents (e.g., ipratropium bromide, oxitropium bromide, tiotropium bromide, CHF 4226 (Chiesi) and glycopyrrolate, but also those described in WO 01/04118, WO 02/51841, WO 02/53564, WO 03/00840, WO 03/87094, WO 04/05285, WO 02/00652, WO 03/53966, EP 424021, U.S. Pat. No. 5,171,744, U.S. Pat. No. 3,714,357, U.S. Pat. No. 5,171,744, WO 03/33495 and WO 04/018422); and beta-2-adrenoceptor agonists such as albuterol (salbutamol), metaproterenol, terbutaline, salmeterol, fenoterol, procaterol, and especially, formoterol and pharmaceutically acceptable salts thereof, and calpain inhibitors. Further suitable beta-2-adrenoreceptor agonists include compounds such as those described in JP 05025045, US 2002/0055651, WO 93/18007, WO 99/64035, WO 01/42193, WO 01/83462, WO 02/066422, WO 02/070490, WO 02/076933, WO 03/24439, WO 03/72539, WO 03/42160, WO 03/91204, WO 03/42164, WO 03/99764, WO 04/11416, WO 04/16578, WO 04/22547, WO 04/32921, WO 04/33412, WO 04/37773, WO 04/37807, WO 04/39762, WO 04/39766, WO 04/45618 and WO 04/46083.

Exemplary co-therapeutic antihistamine drug substances include cetirizine hydrochloride, acetaminophen, clemastine fumarate, promethazine, loratidine, desloratidine, diphenhydramine and fexofenadine hydrochloride, activastine, astemizole, azelastine, ebastine, epinastine, mizolastine and tefenadine as well as those disclosed in JP 2004107299, WO 03/99807 and WO 04/26841.

Combinations of calpain inhibitors and one or more steroids, beta-2 agonists, PDE4 inhibitors or LTD4 antagonists can be used, for example, in the treatment of COPD or asthma. Combinations of calpain inhibitors and anticholinergic or antimuscarinic agents, PDE4 inhibitors, dopamine receptor agonists or LTB4 antagonists can be used, for example, in the treatment of asthma or COPD.

Other useful combinations of calpain inhibitors and for use in the methods described herein are those with other antagonists of chemokine receptors, e.g., CCR-1, CCR-2, CCR-4, CCR-5, CCR-6, CCR-7, CCR-8, CCR-9 and CCR10, CXCR1, CXCR2, CXCR3, CXCR4, CXCR5, or CCR-5 antagonists such as Schering-Plough antagonists SC-351125, SCH-55700 and SCH-D, Takeda antagonists such as N-[[4-[[[6,7-dihydro-2-(4-methylphenyl)-5H-benzo-cyclohepten-8-yl]carbony-1]amino]phenyl]-methyl]tetrahydro-N,N-dimethyl-2H-pyran-4-aminium chloride (TAK-770), CCR-5 antagonists described in U.S. Pat. No. 6,166,037 (claims 18 and 19), WO 00/66558 (claim 8), and WO 00/66559 (claim 9), WO 04/018425 and WO 04/026873.

In some embodiments, the invention also provides a method for the treatment of a condition mediated by CCR-3, for example an inflammatory or allergic condition, an inflammation-related disease or disorder, which comprises administering to a subject (e.g., a human subject), in need thereof an effective amount of a compound of formula Ia or Ib in a free or pharmaceutically acceptable salt form as hereinbefore described. In another aspect the invention provides the use of a compound of formula Ia or Ib, in free or pharmaceutically acceptable salt form, as hereinbefore described for the manufacture of a medicament for the treatment of a condition mediated by CCR-3, e.g., an inflammatory or allergic condition or an inflammation-related disease or disorder.

Other useful combinations of calpain inhibitors and for use in the methods described herein are those for use with treatments for asthma and asthma related conditions. For example, the calpain inhibitors and methods described herein can be used to treat, inhibit, arrest, delay or reduce inflammation in a subject alone or in conjunction with a short-acting bronchodilator (e.g., via an inhaler) to alleviate symptoms of asthma attacks or exacerbations. Short-acting bronchodilators include beta-2 agonists and anticholinergics. Other agents that are suitable for use with the methods described herein include corticosteroids, steroid anti-inflammatory agents, beta2-agonist bronchodilators. Cromolyn sodium and nedocromil are mild to moderate anti-inflammatory drugs, and can be the initial choice for long-term therapy in children. They are also used preventively, before exercise or before exposure to a trigger. Long-acting beta-2 agonists can also be used just prior to exercise to prevent exercise-induced bronchospasm. Leukotriene modifiers can be considered an alternative to low doses of inhaled corticosteroids or to cromolyn sodium and nedocromil for subjects aged 12 years or older with mild persistent asthma. Omalizumab is an injectable immunotherapy for moderate to severe allergic asthmatics for whom corticosteroids fail to control symptoms. See, NIH Practical Guide to the Diagnosis and Management of Asthma, NIH Publication No. 97-4053, National Heart, Lung, and Blood Institute (Bethesda, Md., 1997).

Calpain inhibitors and the methods described herein can be used in conjunction with asthma treatments are directed to (i) preventing, reducing or alleviating the inflammatory response of an asthma attack (e.g., with corticosteroids) and/or (ii) relaxing the muscles that surround the bronchial tubes to thereby open the airway.

Modes of Administration

The calpain inhibitors described herein can be administered by any appropriate route, e.g., orally, for example in the form of a tablet or capsule; parenterally, for example intravenously; by inhalation, for example in the treatment of inflammation-related disease or disorder; intranasally, for example in the treatment of allergic rhinitis; topically to the skin, e.g., in the treatment of atopic dermatitis; or rectally, e.g., in the treatment of inflammatory bowel disease.

In a further aspect, the invention also provides a pharmaceutical composition comprising a calpain inhibitor in free or pharmaceutically acceptable salt form, optionally together with a pharmaceutically acceptable diluent or carrier therefor. The composition can contain a co-therapeutic agent such as an anti-inflammatory bronchodilatory or antihistamine drug as described herein. Such compositions can be prepared using conventional diluents or excipients and techniques known in the galenic art. Thus oral dosage forms can include tablets and capsules. Formulations for topical administration can take the form of creams, ointments, gels or transdermal delivery systems, e.g., patches. Compositions for inhalation can comprise aerosol or other atomizable formulations or dry powder formulations.

Routes of Administration

Calpain inhibitors for the treatment of an inflammation-related disease or disorder with the methods described herein can be administered by parenteral, topical, intravenous, oral, subcutaneous, intraarterial, intracranial, intraperitoneal, intranasal or intramuscular means for prophylactic as inhalants for small chemical molecule, siRNA molecule or antibody preparations targeting an inflammatory-related disease or disorder, and/or therapeutic treatment. Exemplary routes of administration of the calpain inhibitors for use in the methods described herein comprise subcutaneous delivery, intramuscular or intrapulmonary injection, intraveinous delivery, oral delivery, delivery by inhalation, topical delivery or rectal delivery. In some methods, agents can be injected directly into a tissue where inflammation has occurred or is at risk of occurring is found, for example intrapulmonary injection or inhalation delivery. Agents of the invention can optionally be administered in combination with other agents that are at least partly effective in treating various diseases including various immune-related diseases.

Calpain inhibitors have also been designed to be cell permeable. U.S. patent application Ser. No. 11/574,095 describes alpha-keto carbonyl calpain inhibitors suitable for use with the compositions and methods described herein (Lescop et al, 2005. Bioorg Med Chem Lett. 15, 5176-5181). Myodur, another cell permeable calpain inhibitor, is a leupeptin analog attached to carnitine carrier molecule. Carnitine is a compound present in skeletal muscle involved in the transfer of fatty acids across mitochondrial membranes and can prevent calpain activity inside cells.

The calpain inhibitors described herein can also be administered with the aid of cell permeating agents. Nucleic acid molecules can be administered to cells by a variety of methods known to those of skill in the art, including, but not restricted to, administration within formulations that comprise a calpain inhibitor and a cell permeating agent one or more additional components, such as a pharmaceutically acceptable carrier, diluent, excipient, adjuvant, emulsifier, buffer, stabilizer, preservative, and the like.

In certain embodiments, the calpain inhibitor can be encapsulated in liposomes, administered by iontophoresis, or incorporated into other vehicles, such as hydrogels, cyclodextrins, biodegradable nanocapsules, bioadhesive microspheres, or proteinaceous vectors (see e.g., O'Hare and Normand, International PCT Publication No. WO 00/53722). In other embodiment, the calpain inhibitor can be linked to a cell permeable peptide.

Methods for the delivery of nucleic acid molecules are described in Akhtar et al., Trends Cell Bio. 2:139, 1992; “Delivery Strategies for Antisense Oligonucleotide Therapeutics,” ed. Akhtar, 1995; Maurer et al., Mol. Membr. Biol. 16:129-140, 1999; Hofland and Huang, Handb. Exp. Pharmacol. 137:165-192, 1999; and Lee et al., ACS Symp. Ser. 752:184-192, 2000. Beigelman et al., U.S. Pat. No. 6,395,713 and Sullivan et al., PCT WO 94/02595 further describe the general methods for delivery of nucleic acid molecules. These protocols can be utilized for the delivery of virtually any nucleic acid molecule. Nucleic acid molecules can be administered to cells by a variety of methods known to those of skill in the art, including, but not restricted to, encapsulation in liposomes, by iontophoresis, or by incorporation into other vehicles, such as biodegradable polymers, hydrogels, cyclodextrins (see for example, Gonzalez et al., Bioconjugate Chem. 10:1068-1074, 1999; Wang et al., International PCT Publication Nos. WO 03/47518 and WO 03/46185), poly(lactic-co-glycolic)ac-id (PLGA) and PLCA microspheres (see for example, U.S. Pat. No. 6,447,796 and U.S. Patent Application Publication No. US 2002130430), biodegradable nanocapsules, and bioadhesive microspheres, or by proteinaceous vectors (O'Hare and Normand, International PCT Publication No. WO 00/53722). In certain embodiments, the calpain inhibitor can be encapsulated in an endosome.

Within additional embodiments of the invention, a cell permeating agent can be a cell permeating peptide selected or rationally designed to comprise an amphipathic amino acid sequence. For example, a cell permeating agent can comprise a plurality of non-polar or hydrophobic amino acid residues that form a hydrophobic sequence domain or motif, linked to a plurality of charged amino acid residues that form a charged sequence domain or motif, yielding an amphipathic peptide. The calpain inhibitors described herein can be linked to the calpain inhibitors described herein by any method known in the art. Such linkages include, but are not limited to, peptide bonds, covalent bonds, electrostatic interactions, hydrophobic interactions or hydrogen bonds. Methods generating cell permeable fusion proteins are described in U.S. patent application Ser. No. 10/232,410. Methods for delivering nucleic acid with cell permeating peptides are discussed in U.S. patent application Ser. Nos. 10/831,342 and 10/722,176. Methods for the transport of biological molecules and particles are discussed in U.S. patent application Ser. No. 10/541,594

In other embodiments, the polynucleotide delivery-enhancing polypeptide is selected to comprise a protein transduction domain or motif, and a fusogenic peptide domain or motif. A protein transduction domain is a peptide sequence that is able to insert into and, in some cases, transit through the membrane of cells. A fusogenic peptide is a peptide that is able destabilize a lipid membrane, for example a plasma membrane or membrane surrounding an endosome, which can be enhanced at low pH. Exemplary fusogenic domains or motifs are found in a broad diversity of viral fusion proteins and in other proteins, for example fibroblast growth factor 4 (FGF4).

Cell permeating peptides that can be linked to the calpain inhibitors described herein can be a protein transduction domain to facilitate entry of the calpain inhibitor into a cell through the plasma membrane. Examples of protein transduction domains for optional incorporation into polypeptides capable of inhibiting calpain activity include, but are not limited to: TAT protein transduction domain (PTD) (SEQ ID NO:1) KRRQRRR; Penetratin PTD (SEQ ID NO:2) RQIKIWFQNRRMKWKK; VP22 PTD (SEQ ID NO:3) DAATATRGRSAASRPTERPRAPAR SASRPRRPVD; Kaposi FGF signal sequences (SEQ ID NO:4) AAVALLPAVLLALLAP, and SEQ ID NO:5) AAVLLPVLLPVLLAAP; Human .beta.3 integrin signal sequence (SEQ ID NO: 6) VTVLALGALAGVGVG; gp41 fusion sequence (SEQ ID NO:7) GALFLGWLGAAG STMGA; Caiman crocodylus Ig(v) light chain (SEQ ID NO: 8) MGLGLHLLVLAAALQGA; hCT-derived peptide (SEQ ID NO:9) LGTYTQDFNKFHT FPQTAIGVGAP; Transportan (SEQ ID NO:10) GWTLNSAGYLLKINLKALAA LAKKIL; Loligomer (SEQ ID NO:11) TPPKKKRKVEDPKKKK; Arginine peptide (SEQ ID NO:12) RRRRRRR; and 12. Amphiphilic model peptide (SEQ ID NO:13) KLALKLALKALKAALKLA.

Yet additional cell permeating polypeptides that can be linked used to deliver the calpain inhibitors described herein can be selected from the following peptides:

(SEQ ID NO: 14)
WWETWKPFQCRICMRNFSTRQARRNHRRRHR;
(SEQ ID NO: 15)
GKINLKALAALAKKIL,
(SEQ ID NO: 16)
RVIRVWFQNKRCKDKK,
(SEQ ID NO: 17)
GRKKRRQRRRPPQGRKKRRQRRRPPQGRKKRRQRRRPPQ,
or
(SEQ ID NO: 18)
GEQIAQLIAGYIDIILKKKKSK

Thus, in one aspect, methods for treating a subject having to at risk of having an inflammatory disease or disorder can be performed by contacting a cell of the subject with an effective amount of a fusion protein having a first portion and a second portion, the first portion including a cell permeating peptide capable of delivering the fusion protein into the cell and the second portion including a calpain inhibitor or a biologically active variant thereof.

Enhancing Pulmonary Delivery of Therapeutic Agents

Lung diseases comprise a spectrum of manifestations and etiologies, and can be difficult to treat with systemic administration of potential therapeutics. Pulmonary administration of therapeutic compositions comprised of low molecular weight drugs has been observed, for example, beta-androgenic antagonists to treat asthma. Other therapeutic agents that are active in the lungs have been administered systemically and targeted via pulmonary absorption.

The anatomy and physiology of the lung presents several barriers to pulmonary administration. After passing through the nose or mouth, inhaled air (and any particles contained therein) moves into the respiratory tree, which is composed of numerous dichotomous branches between the trachea and the alveoli. Bronchi, bronchioles, and terminal bronchioles comprise the conducting zone. The more distal levels of branching form the transitional and respiratory zones, comprised of respiratory bronchioles, alveolar ducts, and alveoli, is where gas exchange and pulmonary absorption occur.

The air-blood barrier is comprised of the alveolar epithelium, the capillary endothelium, and the lymph-filled interstitial space separating these two cell layers. In the alveolar epithelium, adjacent cells overlap and are bound by non-leaky tight junctions, which, in conjunction with the non-leaky single cell layer comprising the capillary endothelium, limits the movement of fluids, cells, salts, proteins, and numerous other macromolecules from the blood and intercellular spaces into the lumen of the alveoli. Most molecules, including proteins and polypeptides, must be actively or passively transported across this barrier in the absence of lung injury. Also, mucosal secretions from epithelial cells and cilia provide additional physical barriers to the delivery of a potential therapeutic.

Other cell types present in the alveolar lumen and in the interstitial space separating the alveolar epithelium from the capillary endothelium can also serve as barriers for delivery. Alveolar macrophages migrate from the blood across the air-blood barrier. Additionally, other cell types, such as neutrophils and lymphocytes, can move into the alveoli from the blood in response to infection.

In one embodiment of the invention, calpain inhibitors can be directly delivered to the respiratory tract of a subject suffering from, or at risk of suffering from an airway inflammation related disease or disorder. A number of general methods have been described for delivering medically important molecules, including small molecules, nucleic acids, and/or protein or peptide compositions, in an effort to improve bioavailability and/or to target delivery to locations within the body. Such methods include the use of prodrugs, encapsulation into liposomes or other particles, co-administration in uptake enhancing formulations, and targeting to specific tissues. For review see, e.g., Critical Reviews in Therapeutic Drug Carrier Systems, Stephen D. Bruck, ed., CRC Press, 1991. In the case of cytokines such as IL-2, pulmonary delivery has relied upon both inhalation of free cytokine (alone or in combination with intravenous delivery of additional cytokine), and inhalation of liposomal formulations. See, e.g., Enk et al., Cancer 88: 2042-46 (2000); Khanna et al., J. Pharm. Pharmacol. 49: 960-71 (1997). Such delivery modes can provide high cytokine levels within the lung, but relatively modest systemic cytokine levels.

The calpain inhibitors can be formulated as aerosols for topical application, such as by inhalation (see, U.S. Pat. Nos. 4,044,126, 4,414,209, and 4,364,923, which describe aerosols for delivery of a steroid useful for treatment inflammatory diseases, e.g., asthma). These formulations for administration to the respiratory tract can be in the form of an aerosol or solution for a nebulizer, or as a microfine powder for insufflation, alone or in combination with an inert carrier such as lactose. In such a case, the particles of the formulation can have diameters of less than about 50 microns or less than about 10 microns.

When the calpain inhibitor composition comprises an aerosol formulation, it can contain, for example, a hydro-fluoro-alkane (HFA) propellant such as HFA134a or HFA227 or a mixture of these, and can contain one or more co-solvents known in the art such as ethanol (up to 20% by weight), and/or one or more surfactants such as oleic acid or sorbitan trioleate, and/or one or more bulking agents such as lactose. When the composition comprises a dry powder formulation, it can contain, for example, a calpain inhibitor, optionally together with a diluent or carrier, such as lactose, of a given particle size (or particle size distribution) and a compound that helps to protect against product performance deterioration due to moisture e.g., magnesium stearate. When the composition comprises a nebulized formulation, it can contain, for example, a calpain inhibitor dissolved, or suspended, in a vehicle containing water, a co-solvent such as ethanol or propylene glycol and a stabilizer, which can be a surfactant.

In some embodiments, the invention can comprise (A) a calpain inhibitor in inhalable form, e.g., in an aerosol or other atomizable composition or in inhalable particulate, e.g., micronized form; (B) an inhalable medicament comprising a calpain inhibitor in inhalable form; (C) a pharmaceutical product comprising such a calpain inhibitor in inhalable form in association with an inhalation device; or (D) an inhalation device containing a calpain inhibitor in inhalable form.

Dosages of calpain inhibitors employed in practicing the invention described herein can be varied depending, for example, on the condition to be treated, the effect desired and the mode of administration. Suitable daily dosages for administration by inhalation are of the order of 0.001 to 30 mg/kg while for oral administration suitable daily doses are of the order of 0.001 to 100 mg/kg. Dosage ranges can also be determined experimentally in mice and extrapolated to appropriate dosage ranges for use in humans.

The compositions of the invention can be formulated in any suitable manner for delivery to airway epithelia. Suitable formulations include dry particulate and liquid formulations. Dry formulations include freeze dried and lyophilized powders, which are suited for aerosol delivery to the sinuses or lung, or for long term storage followed by reconstitution in a suitable diluent prior to administration. The amount of biologically active component to be delivered can depend on many factors, including the effect to be achieved, the type of organism to which the composition can be delivered, delivery route, dosage regimen, and the age, health, and sex of the organism. As such, the dosage can be left to the ordinarily skilled artisan's discretion. Additionally, particle size can be controlled to achieve optimal delivery to a specific region of the organ (e.g., the lung). Particle sizes can be between about 1 .mu.m and about 20 .mu.m, between about 1 .mu.m and about 10 .mu.m, between about 2 .mu.m and about 7 .mu.m, and between about 3 .mu.m and about 5 .mu.m.

For example, methods used for pulmonary delivery of Tobramycin for Inhalation (TOBI) can be used for pulmonary delivery of the calpain inhibitors and compositions described herein. In contrast to injectable preparations of tobramycin, TOBI contains no stabilizing addition of antioxidants, which, upon inhalation, can trigger fits of coughing or asthma. Accordingly, in one embodiment, calpain inhibitors and compositions described herein can be aerosolized and inhaled by means of a nebulizer. The efficiency of the pulmonary administration can depend on the size of the particles of the aerosol. The efficiency of administration can also depend on the device used. In one embodiment, the jet nebulizer PARI LC PLUS.™. in combination with the compressor Pari Master.™. (both marketed by PARI) can be used for pulmonary delivery of the calpain inhibitors and compositions described herein.

Certain modes for delivering medically important molecules (e.g., oral, nasopharyngeal, oropharyngeal, pulmonary, buccal, sublingual, mucosal, vaginal, or rectal delivery modes) require that the molecule(s) of interest be delivered across polarized cells (e.g., epithelial cells) that have two distinct surfaces. In the case of pulmonary epithelium, these surfaces are referred to as the apical surface, which is exposed to the aqueous or gaseous medium in which the molecule(s) of interest can be delivered to the subject; and the opposing basolateral (also known as basal lateral) side that rests upon and is supported by an underlying basement membrane, and that can provide access to the interstitial spaces and the general circulation. Cell junctions between adjacent epithelial cells separate the apical and basolateral sides of an individual epithelial cell. The biological methods that provide and maintain such cellular polarity can also act to limit bioavailability of molecules delivered by these modes.

Pulmonary delivery of therapeutic agents in subjects suffering from, or at risk of suffering from an inflammation-related disease or disorder can be limited by the barrier presented by the polarized epithelium lining the pulmonary system. Such epithelial cells are said to be polarized; that is, they are capable of generating gradients between the compartments they separate due to these distinct surfaces having distinct transport and permeability characteristics. (for reviews, see Knust, Curr. Op. Genet. Develop. 10:471-475, 2000; Matter, Curr. Op. Genet. Develop. 10:R39-R42, 2000; Yeaman et al., Physiol. Rev. 79:73-98, 1999).

The calpain inhibitors described herein can be administered to the afflicted patient by means of a pharmaceutical delivery system for the inhalation route. The compounds may be formulated in a form suitable for administration by inhalation. The pharmaceutical delivery system is one that is suitable for respiratory therapy by topical administration of calpain inhibitors thereof to mucosal linings of the bronchi. This invention can utilize a system that depends on the power of a compressed gas to expel the calpain inhibitors from a container. An aerosol or pressurized package can be employed for this purpose.

As used herein, the term “aerosol” is used in its conventional sense as referring to very fine liquid or solid particles carries by a propellant gas under pressure to a site of therapeutic application. When a pharmaceutical aerosol is employed in this invention, the aerosol contains the therapeutically active compound, which can be dissolved, suspended, or emulsified in a mixture of a fluid carrier and a propellant. The aerosol can be in the form of a solution, suspension, emulsion, powder, or semi-solid preparation. Aerosols employed in the present invention are intended for administration as fine, solid particles or as liquid mists via the respiratory tract of a patient. Various types of propellants known to one of skill in the art can be utilized. Examples of suitable propellants include, but is not limited to, hydrocarbons or other suitable gas. In the case of the pressurized aerosol, the dosage unit may be determined by providing a value to deliver a metered amount.

The present invention can also be carried out with a nebulizer, which is an instrument that generates very fine liquid particles of substantially uniform size in a gas. In one embodiment, a liquid containing the calpain inhibitors is dispersed as droplets. The small droplets can be carried by a current of air through an outlet tube of the nebulizer. The resulting mist penetrates into the respiratory tract of the patient.

A powder composition containing calpain inhibitors or analogs thereof, with or without a lubricant, carrier, or propellant, can be administered to a mammal in need of therapy. This embodiment of the invention can be carried out with a conventional device for administering a powder pharmaceutical composition by inhalation. For example, a powder mixture of the compound and a suitable powder base such as lactose or starch may be presented in unit dosage form in for example capsular or cartridges, e.g. gelatin, or blister packs, from which the powder may be administered with the aid of an inhaler.

The appropriate dosage level will also vary depending on a number of factors including the nature of the subject to be treated, the particular nature of the inflammatory condition to be treated and its severity, the nature of the calpain inhibitors used as active ingredient, the mode of administration, the formulation, and the judgment of the practitioner. For example, when antibodies are administered by themselves such as anti-calpain in an injectable formulation, the dosages can be in the range of 20 mg/kg to about 40 mg/kg at a single dosage. Repeated administration over a period of days may be required or administration by intravenous means may be continuous. For chronic conditions, administration may be continued for longer periods as necessary.

Efficacy of the dosing regime can be determined by assessing for improved lung function in the patient. This assessment may include viscoelasticity measurements of sputum, improvements in pulmonary function, including improvements in forced exploratory volume of sputum and maximal midexpiratory flow rate. The aforementioned therapeutic regime can be given in conjunction with adjunct therapies such as antibiotics or other current therapies for the treatment of an inflammatory condition. If antibiotics are co-administered as part of the patient s therapy, bacterial quantitation following therapy can be included to assess the efficacy of the treatment by decreased bacterial growth, indicating decreased viscosity of mucus or sputum and increase of the mucus or sputum lung clearance.

Pulmonary function tests, as well as diagnostic tests for the clinical progression of an inflammatory condition, are known to those individuals with skill in this art. Standard pulmonary function tests include airway resistance (AR); forced vital capacity (FVC); forced expiratory volume in 1 second (FEV(1)); forced midexpiratory flow; and peak expiratory flow rate (PEFR). Other pulmonary function tests include blood gas analysis; responses to medication; challenge and exercise testing; measurements of respiratory muscle strength; fibro-optic airway examination; and the like. Some basic procedures for studying the properties of mucus include rheology, e.g. with the use of a magnetic microrheometer; adhesivity to characterize the forces of attraction between an adherent surface and an adhesive system by measuring the contact angle between a mucus drop and a surface. Mucus transport by cilia can be studied using conventional techniques, as well as direct measurement, i.e. in situ mucus clearance. Transepithelial potential difference, the net result of the activity of the ion-transport system of the pulmonary epithelium, can be measured using appropriate microelectrodes. Quantitative morphology methods may be used to characterize the epithelial surface condition.

All formulations for aerosol, trans-thoracic, instillation, intravenous and/or other administration can be formulated in dosages suitable for administration. Diagnostic and/or pharmaceutical compositions suitable for use in the present invention include compositions wherein the calpain inhibitors are present in an effective amount, i.e., in a diagnostically and/or pharmaceutically effective amount.

The effective amount when referring to a composition comprising a calpain inhibitor will can mean the dose ranges, modes of administration, formulations, etc., that have been recommended or approved by any of the various regulatory or advisory organizations in the medical or pharmaceutical arts (e.g., FDA, AMA) or by the manufacturer or supplier. The effective amount when referring to producing a benefit in treating a pulmonary condition, such as an inflammatory condition, can be the amount that achieves clinical lung volume reduction recommended or approved by any of the various regulatory or advisory organizations in the medical or surgical arts (e.g., FDA, AMA) or by the manufacturer or supplier.

A person of ordinary skill using techniques known in the art can determine the effective amount of a calpain inhibitor to be administered. The effective amount may depend on the calpain inhibitor to be used, and can be deduced from known data. In some embodiments, dosages can be at least about 0.001 .mu.g/kg/body weight, at least about 0.005 .mu.g/kg/body weight, at least about 0.01 .mu.g/kg/body weight, at least about 0.05 .mu.g/kg/body weight, or at least about 0.1 .mu.g/kg/body weight. In some embodiment, dosages can be less than about 0.05 mg/kg/body weight, less than about 0.1 mg/kg/body weight, less than about 0.5 mg/kg/body weight, less than about 1 mg/kg/body weight, less than about 2 mg/kg/body weight, less than about 3 mg/kg/body weight, or less than about 5 mg/kg/body weight of a composition of the invention. In some embodiment, dosages can be less than about 10 mg/kg/body weight, less than about 25 mg/kg/body weight, less than about 50 mg/kg/body weight, less than about 75 mg/kg/body weight, less than about 100 mg/kg/body weight, less than about 150 mg/kg/body weight, or less than about 200 mg/kg/body weight of a calpain inhibitor as described herein.

The dosage of the calpain inhibitor may vary depending on the moieties used and their known biological properties. For example, a formulation comprising at least about 0.00001%, at least about 0.0001%, at least about 0.001%, at least about 0.01%, at least about 0.1%, at least about 1%, at least about 2%, at least about 3%, at least about 4%, at least about 5%, at least about 8%, at least about 10%, at least about 12%, or at least about 15% of a calpain inhibitor may be used (e.g., in saline solution, for instance about 0.8%, about 0.9%, about 1%, or about 1.2% saline).

Further, the effective amount of a calpain inhibitor for use in humans can be determined from animal models, e.g., mice, rabbits, dogs, sheep, or pigs. For example, emphysema can be induced in C57BL/6 mice by administering nebulized porcine pancreatic elastase (about 30 IU/day for about 6 days), as described, for instance, in Ingenito et al., Tissue heterogeneity in the mouse lung: effects of elastase treatment, Articles in Press. J Appl Physiol (Mar. 12, 2004). 10.1152/japplphysiol.01246.2003. Similarly, emphysema-like conditions may be induced in sheep exposed to papain (inhalation of about 7,000 units/week for four consecutive weeks). Emphysema can also be induced in animal models by exposure to cadmium chloride, high concentrations of oxygen, and/or cigarette smoke. Ingenito, et al., “Bronchoscopic lung volume reduction using tissue engineering principles”, American Journal of Respiratory and Critical Care Medicine, Vol. 167 pgs. 771-778 (2003). A dose suitable for reducing inflammation in humans can be formulated based on doses found to be effective in animal models. Other techniques would be apparent to one of ordinary skill in the art.

The exact dosage of a calpain inhibitor can be determined by the practitioner, in light of factors related to the subject in need of diagnosis and/or treatment. Factors which may be taken into account include the severity or extent of the pulmonary condition, the general health of the subject, age, weight, and diet of the subject, as well as the timing and frequency of administration, other diagnostic and/or therapeutic techniques available and/or desirable to the subject, and/or being used by the subject, as well as reaction sensitivities, allergies, tolerance and/or response to the composition(s) of the present invention.

Molecules are trafficked into, out of, and within a cell (e.g., a polarized cell) by various means, and can confer bioavailability to a molecule delivered by oral, nasopharyngeal, oropharyngeal, pulmonary, buccal, sublingual, mucosal, vaginal, or rectal delivery modes. Methods of delivery of the calpain inhibitors for use in the methods described herein to airway epithelia can be by active transport (e.g., by energy-dependent carriage of substances across a cell membrane), endocytosis (cellular internalization of molecules, i.e., processes in which cells take in molecules from their environment, passively or actively), exocytosis (e.g., processes in which molecules are passively or actively moved from the interior of a cell into the medium surrounding the cell), transcytosis (e.g., processes in which molecules are transported from one surface of a cell to another), paracytosis (e.g., processes in which molecules are transferred through the interstices between cells), or receptor mediated endocytosis (e.g., receptor-mediated internalization of molecules, viruses and bacteria).

Compositions adapted to provide delivery of therapeutic, diagnostic, prophylactic, or imaging molecules into and/or across polarized cells have been described. See, e.g., International Publication No. WO02/28408, which is hereby incorporated by reference in its entirety, including all tables, figures and claims. Such methods comprise associating the therapeutic, diagnostic, prophylactic, or imaging molecules with targeting elements directed to a molecule expressed on the surface of epithelial cells that mediate transport into or across such cells. Numerous molecules are known to enter or exit biological systems by binding to a component that mediates transport of the molecule to or from the cell surface. Examples of such molecules include toxins such as diphtheria toxin, pseudomonas toxin, cholera toxin, ricin, abrin, concanavalin A; certain viruses (Rous sarcoma virus, adenovirus, etc.); transferrin; low density lipoprotein; transcobalamin (vitamin B12); hormones and growth factors such as insulin, epidermal growth factor, growth hormone, thyroid stimulating factor, calcitonin, glucagon, prolactin, lutenizing hormone, thyroid hormone, platelet derived growth factor, and VEGFs; and antibodies such as IgA, and IgM.

In some embodiments, calpain inhibitors can be targeted to epithelial cells by association or linkage with a targeting moiety. Exemplary targeting moieties include, but are not limited to, receptors such as pIgR, a scavenger receptor, a GPI-linked protein, transferrin receptor, vitamin B12 receptor, FcRn, intergrins, low density lipoprotein receptor; cargo carrier fragments such as pIgR stalk, members of the PGDF, FGF, and VEGF receptor families (e.g., Flt-1, Flk-1, Flt-4, FGFR1, FGFR2, FGFR3, FGFR4), and surface antigens. This list is not meant to be limiting. Other receptors include scavenger receptors (e.g., CLA-I/SR-B1, CD-36, intrinsic factor, cubilin, megalin, GP 330), p75NTR (Neurotrophin receptor), Leptin receptor, TGF-beta receptor, TGF beta receptor II, reduced folate carrier, Mannose-6-phosphate receptor, CaR (calcium receptor), A2b adenosine receptor, IGF-I receptor, IGF-II receptor, ebnerin (taste), 67 kDa laminin receptor, laminin receptor precursor (LRP), TGF-beta receptor III, transcobalamin receptor, HGF-SF (hepatocyte growth factor/scatter factor, c-met) receptor, CD4 receptor, TGF-beta I receptor, c-erbB (EGF receptor), ASGP-R (asialoglycoprotein receptor), LRP (low density lipoprotein receptor related protein) receptor, CFTR (cystic fibrosis transmembrane conductance regulator), sucrose isomaltase, receptors for toxins, viruses, and bacteria (e.g., GM1 ganglioside (cholera toxin), Galactosyl ceramide (HIV), receptor for anthrax protective antigen, CD46 (measles), 85 kDa CSL receptor (cryptosporidium), GD1b (E. coli type II temperature sensitive enterotoxin (LTIIa)), GC-C Guanylyl cyclase (E. coli heat stable enterotoxin (STa)), putative Hepatitis A receptor, Toll-like receptor 5 (TLR5)), transporters/exchangers (e.g., PepT1, ENaC (sodium), GLUT-5, SGLT-1, CaT1 (calcium), EcaC (calcium), NHE 3 (Na+/H+ exchanger)), apolipoproteins (e.g., apolipoprotein A1, A2, A3, A4, A5, B, C1, C2, C3, C4, D, and/or E), aquaporin, high density lipoprotein binding proteins (e.g., ATP binding cassette protein-1, scavenger receptor-BI), viral receptors (e.g., coxsakie adenovirus receptor, .alpha.v integrins, sialic acid-containing glycoproteins, CD4), and proteases (e.g., epitheliasin, Aminopeptidase N, Dipeptidylpeptidase).

Formulations for Oral Administration

Oral pharmaceutical dosage forms can be as a solid, gel or liquid. Examples of solid dosage forms include, but are not limited to tablets, capsules, granules, and bulk powders. More specific examples of oral tablets include compressed, chewable lozenges and tablets that can be enteric-coated, sugar-coated or film-coated. Examples of capsules include hard or soft gelatin capsules. Granules and powders can be provided in non-effervescent or effervescent forms. Each can be combined with other ingredients known to those skilled in the art.

Oral formulations can include excipients, such as pharmaceutical grades of mannitol, lactose, starch, magnesium stearate, sodium saccharine, cellulose, and magnesium carbonate. These compositions can take the form of solutions, suspensions, tablets, pills, capsules, sustained release formulations or powders.

In certain aspects, the calpain inhibitors for use according to the methods described herein can be provided as solid dosage forms as capsules or tablets. The tablets, pills, capsules, troches and the like can optionally contain one or more of the following ingredients, or compounds of a similar nature: a binder; a diluent; a disintegrating agent; a lubricant; a glidant; a sweetening agent; and a flavoring agent.

Examples of binders that can be used include, but are not limited to, microcrystalline cellulose, gum tragacanth, glucose solution, acacia mucilage, gelatin solution, sucrose and starch paste.

Examples of lubricants that can be used include, but are not limited to, talc, starch, magnesium or calcium stearate, lycopodium and stearic acid.

Examples of diluents that can be used include, but are not limited to, lactose, sucrose, starch, kaolin, salt, mannitol and dicalcium phosphate.

Examples of glidants that can be used include, but are not limited to, colloidal silicon dioxide.

Examples of disintegrating agents that can be used include, but are not limited to, crosscarmellose sodium, sodium starch glycolate, alginic acid, corn starch, potato starch, bentonite, methylcellulose, agar and carboxymethylcellulose.

Examples of coloring agents that can be used include, but are not limited to, any of the approved certified water soluble FD and C dyes, mixtures thereof; and water insoluble FD and C dyes suspended on alumina hydrate.

Examples of sweetening agents that can be used include, but are not limited to, sucrose, lactose, mannitol and artificial sweetening agents such as sodium cyclamate and saccharin, and any number of spray-dried flavors.

Examples of flavoring agents that can be used include, but are not limited to, natural flavors extracted from plants such as fruits and synthetic blends of compounds that produce a pleasant sensation, such as, but not limited to peppermint and methyl salicylate.

Examples of wetting agents that can be used include, but are not limited to, propylene glycol monostearate, sorbitan monooleate, diethylene glycol monolaurate and polyoxyethylene lauryl ether.

Examples of anti-emetic coatings that can be used include, but are not limited to, fatty acids, fats, waxes, shellac, ammoniated shellac and cellulose acetate phthalates.

Examples of film coatings that can be used include, but are not limited to, hydroxyethylcellulose, sodium carboxymethylcellulose, polyethylene glycol 4000 and cellulose acetate phthalate.

For oral administration, the salt of the compound can optionally be provided in a composition that protects it from the acidic environment of the stomach. For example, the composition can be formulated in an enteric coating that maintains its integrity in the stomach and releases the active compound in the intestine. The composition can also be formulated in combination with an antacid or other such ingredient.

When the dosage unit form is a capsule, it can optionally additionally comprise a liquid carrier such as a fatty oil. In addition, dosage unit forms can optionally additionally comprise various other materials that modify the physical form of the dosage unit, for example, coatings of sugar and other enteric agents.

Compounds according to the invention described herein can also be administered as a component of an elixir, suspension, syrup, wafer, sprinkle, chewing gum or the like. A syrup can optionally comprise, in addition to the active compounds, sucrose as a sweetening agent and certain preservatives, dyes and colorings and flavors.

The calpain inhibitors for use in the methods described herein can also be mixed with other active materials that do not impair calpain inhibition, or with materials that supplement the calpain inhibition, such as antacids, H2 blockers, and diuretics. For example, if a compound is used for treating an inflammation-related disease or disorder, it can be used with other bronchodilators and antihypertensive agents, respectively.

Examples of pharmaceutically acceptable carriers that can be included in tablets comprising calpain inhibitors of the invention described herein include, but are not limited to binders, lubricants, diluents, disintegrating agents, coloring agents, flavoring agents, and wetting agents. Enteric-coated tablets, because of the enteric-coating, resist the action of stomach acid and dissolve or disintegrate in the neutral or alkaline intestines. Sugar-coated tablets can be compressed tablets to which different layers of pharmaceutically acceptable substances are applied. Film-coated tablets can be compressed tablets that have been coated with polymers or other suitable coating. Multiple compressed tablets can be compressed tablets made by more than one compression cycle utilizing the pharmaceutically acceptable substances previously mentioned. Coloring agents can also be used in tablets. Flavoring and sweetening agents can be used in tablets, and are especially useful in the formation of chewable tablets and lozenges.

Examples of liquid oral dosage forms that can be used include, but are not limited to, aqueous solutions, emulsions, suspensions, solutions and/or suspensions reconstituted from non-effervescent granules and effervescent preparations reconstituted from effervescent granules.

Examples of aqueous solutions that can be used include, but are not limited to, elixirs and syrups, e.g., clear, sweetened, hydroalcoholic preparations. Examples of pharmaceutically acceptable carriers that can be used in elixirs include, but are not limited to solvents. Examples of solvents that can be used include glycerin, sorbitol, ethyl alcohol and syrup. As used herein, syrups refer to concentrated aqueous solutions of a sugar, for example, sucrose. Syrups can optionally further comprise a preservative.

Emulsions refer to two-phase systems in which one liquid is dispersed in the form of small globules throughout another liquid. Emulsions can optionally be oil-in-water or water-in-oil emulsions. Examples of pharmaceutically acceptable carriers that can be used in emulsions include, but are not limited to non-aqueous liquids, emulsifying agents and preservatives.

Examples of pharmaceutically acceptable substances that can be used in non-effervescent granules, to be reconstituted into a liquid oral dosage form, include diluents, sweeteners and wetting agents.

Examples of pharmaceutically acceptable substances that can be used in effervescent granules, to be reconstituted into a liquid oral dosage form, include organic acids and a source of carbon dioxide. Coloring and flavoring agents can optionally be used in the dosage forms described herein.

Examples of preservatives that can be used include glycerin, methyl and propylparaben, benzoic add, sodium benzoate and alcohol. Examples of non-aqueous liquids that can be used in emulsions include mineral oil and cottonseed oil. Examples of emulsifying agents that can be used include gelatin, acacia, tragacanth, bentonite, and surfactants such as polyoxyethylene sorbitan monooleate. Examples of suspending agents that can be used include sodium carboxymethylcellulose, pectin, tragacanth, Veegum and acacia. Diluents include lactose and sucrose. Sweetening agents include sucrose, syrups, glycerin and artificial sweetening agents such as sodium cyclamate and saccharin. Examples of wetting agents that can be used include propylene glycol monostearate, sorbitan monooleate, diethylene glycol monolaurate and polyoxyethylene lauryl ether. Examples of organic acids that can be used include citric and tartaric acid.

Sources of carbon dioxide that can be used in effervescent compositions include sodium bicarbonate and sodium carbonate. Coloring agents include any of the approved certified water soluble FD and C dyes, and mixtures thereof.

Examples of flavoring agents that can be used include natural flavors extracted from plants such fruits, and synthetic blends of compounds that produce a pleasant taste sensation.

For a solid dosage form, the solution or suspension, in for example propylene carbonate, vegetable oils or triglycerides can be encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, can be diluted with a sufficient quantity of a pharmaceutically acceptable liquid carrier, e.g., water, to be easily measured for administration.

Alternatively, liquid or semi-solid oral formulations can be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. Re 28,819 and 4,358,603.

Injectables, Solutions, and Emulsions

The invention described herein is also directed to compositions designed to administer the calpain inhibitors for use with the methods described herein by parenteral administration (e.g., injection), subcutaneously, intramuscularly or intravenously. Injectables can be prepared in any conventional form, for example as liquid solutions or suspensions, solid forms suitable for solution or suspension in liquid prior to injection, or as emulsions.

For parenteral administration, compositions of the invention can be administered as injectable dosages of a solution or suspension of the substance in a physiologically acceptable diluent with a pharmaceutical carrier that can be a sterile liquid such as water oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. Glycols such as propylene glycol or polyethylene glycol can be used as liquid carriers for injectable solutions. Antibodies can be administered in the form of a depot injection or implant preparation which can be formulated in such a manner as to permit a sustained release of the active ingredient. An exemplary composition comprises monoclonal antibody formulated in aqueous buffer consisting of 50 mM L-histidine, 150 mM NaCl, adjusted to pH 6.0 with HCl.

Compositions can be prepared as injectables, as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as described herein. Langer, Science 249: 1527, 1990; Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient.

Examples of excipients that can be used in conjunction with injectables according to the invention described herein include, but are not limited to water, saline, dextrose, glycerol or ethanol. The injectable compositions can also optionally comprise minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow-release or sustained-release system, such that a constant level of dosage can be maintained (see, e.g., U.S. Pat. No. 3,710,795) is also disclosed herein. The percentage of active compound contained in such parenteral compositions can be dependent on the specific nature thereof, as well as the activity of the compound and the needs of the subject.

Parenteral administration of the formulations includes intravenous, subcutaneous and intramuscular administrations. Preparations for parenteral administration include sterile solutions ready for injection, sterile dry soluble products, such as the lyophilized powders described herein, ready to be combined with a solvent just prior to use, including hypodermic tablets, sterile suspensions ready for injection, sterile dry insoluble products ready to be combined with a vehicle just prior to use and sterile emulsions. The solutions can be aqueous or nonaqueous.

When administered intravenously, examples of suitable carriers include, but are not limited to physiological saline or phosphate buffered saline (PBS), and solutions containing thickening and solubilizing agents, such as glucose, polyethylene glycol, and polypropylene glycol and mixtures thereof.

Examples of pharmaceutically acceptable carriers that can optionally be used in parenteral preparations include, but are not limited to aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles that can optionally be used include Sodium Chloride Injection, Ringers Injection, Isotonic Dextrose Injection, Sterile Water Injection, Dextrose and Lactated Ringers Injection.

Examples of nonaqueous parenteral vehicles that can optionally be used include fixed oils of vegetable origin, cottonseed oil, corn oil, sesame oil and peanut oil.

Antimicrobial agents in bacteriostatic or fungistatic concentrations can be added to parenteral preparations, for example, when the preparations are packaged in multiple-dose containers and thus designed to be stored and multiple aliquots to be removed. Examples of antimicrobial agents that can be used include phenols or cresols, mercurials, benzyl alcohol, chlorobutanol, methyl and propyl p-hydroxybenzoic acid esters, thimerosal, benzalkonium chloride and benzethonium chloride.

Examples of isotonic agents that can be used include sodium chloride and dextrose. Examples of buffers that can be used include phosphate and citrate. Examples of antioxidants that can be used include sodium bisulfate. Examples of local anesthetics that can be used include procaine hydrochloride. Examples of suspending and dispersing agents that can be used include sodium carboxymethylcellulose, hydroxypropyl methylcellulose and polyvinylpyrrolidone. Examples of emulsifying agents that can be used include Polysorbate 80 (TWEEN 800). A sequestering or chelating agent of metal ions include EDTA.

Pharmaceutical carriers can also optionally include ethyl alcohol, polyethylene glycol and propylene glycol for water miscible vehicles and sodium hydroxide, hydrochloric acid, citric acid or lactic acid for pH adjustment.

The concentration of a calpain inhibitor in the parenteral formulation can be adjusted so that an injection administers a pharmaceutically effective amount sufficient to produce the pharmacological effect. The exact concentration of a calpain inhibitor and/or dosage to be used can ultimately depend on the age, weight and condition of the subject or animal as is known in the art.

Unit-dose parenteral preparations can be packaged in an ampoule, a vial or a syringe with a needle. Preparations for parenteral administration can be sterile, as is known and practiced in the art.

Injectables can be designed for local and systemic administration. The calpain inhibitor can be administered at once, or can be divided into a number of smaller doses to be administered at intervals of time. The precise dosage and duration of treatment can be a function of the location of where the composition is parenterally administered, the carrier and other variables that can be determined empirically using known testing protocols or by extrapolation from in vivo or in vitro test data. Concentrations and dosage values can also vary with the age of the individual treated. For any subject, specific dosage regimens can need to be adjusted over time according to the individual need and the professional judgment of the person administering or supervising the administration of the formulations. Hence, the concentration ranges set forth herein are intended to be exemplary and are not intended to limit the scope or practice of the claimed formulations.

The calpain inhibitor can optionally be suspended in micronized or other suitable form or can be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the disease state and can be empirically determined.

Lyophilized Powders

The calpain inhibitors for use with the methods described herein can also be prepared as lyophilized powders, which can be reconstituted for administration as solutions, emulsions and other mixtures. The lyophilized powders can also be formulated as solids or gels.

Sterile, lyophilized powder can be prepared by dissolving the compound in a sodium phosphate buffer solution containing dextrose or other suitable excipient. Subsequent sterile filtration of the solution followed by lyophilization under standard conditions known to those of skill in the art can be used to provide the formulation. Briefly, the lyophilized powder can optionally be prepared by dissolving dextrose, sorbitol, fructose, corn syrup, xylitol, glycerin, glucose, sucrose or other suitable agent in a suitable buffer, such as citrate, sodium or potassium phosphate or other such buffer known to those of skill in the art. Then, a calpain inhibitor can be added to the resulting mixture and stirred until it dissolves. The resulting mixture can be diluted by adding more buffer to obtain a concentration. The resulting mixture can be sterile filtered or treated to remove particulates and to insure sterility, and apportioned into vials for lyophilization. Each vial can contain a single dosage or multiple dosages of the calpain inhibitor.

Topical Administration

The calpain inhibitors for use with the methods described herein can also be administered as topical mixtures. Topical mixtures can be used for local and systemic administration. The resulting mixture can be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

Topical application can result in transdermal or intradermal delivery. Topical administration can be facilitated by co-administration of the agent with cholera toxin or detoxified derivatives or subunits thereof or other similar bacterial toxins. Glenn et al., Nature 391: 851, 1998. Co-administration can be achieved by using the components as a mixture or as linked molecules obtained by chemical crosslinking or expression as a fusion protein.

For suppositories, binders and carriers include, for example, polyalkylene glycols or triglycerides; such suppositories can be formed from mixtures containing the active ingredient in the range of 0.5% to 10% or 1%-2%.

Alternatively, transdermal delivery can be achieved using a skin patch or using transferosomes. Paul et al., Eur. J. Immunol. 25: 3521-24, 1995; Cevc et al., Biochem. Biophys. Acta 1368: 201-15, 1998.

The calpain inhibitors for use with the methods described herein can also be formulated for local or topical application, such as for topical application to the skin and mucous membranes, such as in the eye, in the form of gels, creams, and lotions and for application to the eye or for intracistemal or intraspinal application. Topical administration can be used for transdermal delivery and also for administration to the eyes or mucosa, or for inhalation therapies. Nasal solutions of the calpain inhibitor alone or in combination with other pharmaceutically acceptable excipients can also be administered.

The pharmaceutical compositions are can be formulated as sterile, substantially isotonic and in full compliance with Good Manufacturing Practice (GMP) regulations of the U.S. Food and Drug Administration.

Formulations and Pharmaceutical Compositions

Calpain inhibitors for use with the methods described herein can be any calpain inhibitor useful in the present compositions and methods and can be administered to a subject (for example, a human subject), in the form of a stereoisomer, prodrug, pharmaceutically acceptable salt, hydrate, solvate, acid salt hydrate, N-oxide or isomorphic crystalline form thereof, or in the form of a pharmaceutical composition where the compound can be mixed with suitable carriers or excipient(s) in a therapeutically effective amount, for example, inflammation.

Pharmaceutically acceptable carriers can be determined in part by the composition being administered, as well as by the method used to administer the composition. Accordingly, there is a wide variety of suitable formulations of pharmaceutical compositions for administering the antibody compositions (see, e.g., REMINGTON: THE SCIENCE AND PRACTICE OF PHARMACY, 20th Ed., Gennaro, A. R. (ed.), Mack Publishing Company, Easton, Pa., 2000, incorporated herein by reference). The pharmaceutical compositions can comprise a differentially expressed protein, agonist or antagonist in a form suitable for administration to a subject. The pharmaceutical compositions are can be formulated as sterile, substantially isotonic and in full compliance with Good Manufacturing Practice (GMP) regulations of the U.S. Food and Drug Administration.

A wide variety of compositions and administration methods can be used in conjunction with the methods described herein. Such compositions can include, in addition to one or more calpain inhibitors, conventional pharmaceutical excipients, and other conventional, pharmaceutically inactive agents. Additionally, the compositions can include active agents in addition to the calpain inhibitors of the invention described herein. These additional active agents can include additional compounds according to the invention, and/or one or more other pharmaceutically active agents.

The compositions can be in gaseous, liquid, semi-liquid or solid form, formulated in a manner suitable for the route of administration to be used. For oral administration, capsules and tablets can be used. For parenteral administration, reconstitution of a lyophilized powder, prepared as described herein, can be used.

Compositions comprising calpain inhibitors of the invention described herein can be administered or co-administered orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, topically, or intrathecally. The compounds and/or compositions according to the invention can also be administered or co-administered in slow release dosage forms.

The calpain inhibitors and compositions comprising them can be administered or co-administered in any conventional dosage form. Co-administration in the context of this invention can mean the administration of more than one therapeutic agent, one of which includes a calpain inhibitor, in the course of a coordinated treatment to achieve an improved clinical outcome. Such co-administration can also be coextensive, that is, occurring during overlapping periods of time.

Solutions or suspensions used for parenteral, intradermal, subcutaneous, or topical application can optionally include one or more of the following components: a sterile diluent, such as water for injection, saline solution, fixed oil, polyethylene glycol, glycerine, propylene glycol or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; agents for the adjustment of tonicity such as sodium chloride or dextrose, and agents for adjusting the acidity or alkalinity of the composition, such as alkaline or acidifying agents or buffers like carbonates, bicarbonates, phosphates, hydrochloric acid, and organic acids like acetic and citric acid. Parenteral preparations can optionally be enclosed in ampules, disposable syringes or single or multiple dose vials made of glass, plastic or other suitable material.

When calpain inhibitors according to the invention described herein exhibit insufficient solubility, methods for solubilizing the compounds can be used. Such methods are known to those of skill in this art, and include, but are not limited to, using co-solvents, such as dimethylsulfoxide (DMSO), using surfactants, such as TWEEN, or dissolution in aqueous sodium bicarbonate. Derivatives of the compounds, such as prodrugs of the compounds can also be used in formulating effective pharmaceutical compositions.

Upon mixing or adding calpain inhibitors according to the methods and compositions described herein to a composition, a solution, suspension, emulsion or the like can be formed. The form of the resulting composition can depend upon a number of factors, including the intended mode of administration, and the solubility of the compound in the selected carrier or vehicle. The effective concentration needed to ameliorate the disease being treated can be empirically determined.

Compositions described herein can be provided for administration to humans and animals in unit dosage forms, such as tablets, capsules, pills, powders, dry powders for inhalers, granules, sterile parenteral solutions or suspensions, and oral solutions or suspensions, and oil-water emulsions containing suitable quantities of the compounds, the pharmaceutically acceptable salts, and the sodium salts, thereof. The pharmaceutically therapeutically active compounds and derivatives thereof are can be formulated and administered in unit-dosage forms or multiple-dosage forms. Unit-dose forms can be physically discrete units suitable for human and animal subjects and packaged individually as is known in the art. Each unit-dose can contain a predetermined quantity of the therapeutically active compound sufficient to produce a therapeutic effect, in association with the pharmaceutical carrier, vehicle or diluent. Examples of unit-dose forms include ampoules and syringes individually packaged tablet or capsule. Unit-dose forms can be administered in fractions or multiples thereof. A multiple-dose form can be a plurality of identical unit-dosage forms packaged in a single container to be administered in segregated unit-dose form. Examples of multiple-dose forms include vials, bottles of tablets or capsules or bottles of pint or gallons. Hence, multiple dose form can be a multiple of unit-doses that are not segregated in packaging.

In addition to one or more calpain inhibitors, the composition for use with the methods described herein can comprise: a diluent such as lactose, sucrose, dicalcium phosphate, or carboxymethylcellulose; a lubricant, such as magnesium stearate, calcium stearate and talc; and a binder such as starch, natural gums, such as gum acaciagelatin, glucose, molasses, polyinylpyrrolidine, celluloses and derivatives thereof, povidone, crospovidones and other such binders known to those of skill in the art. Liquid pharmaceutically administrable compositions can, for example, be prepared by dissolving, dispersing, or otherwise mixing an active compound as described herein and optional pharmaceutical adjuvants in a carrier, such as, for example, water, saline, aqueous dextrose, glycerol, glycols, ethanol, and the like, to form a solution or suspension. The pharmaceutical composition to be administered can also contain minor amounts of auxiliary substances such as wetting agents, emulsifying agents, or solubilizing agents, pH buffering agents and the like, for example, acetate, sodium citrate, cyclodextrine derivatives, sorbitan monolaurate, triethanolamine sodium acetate, triethanolamine oleate, and other such agents. Actual methods of preparing such dosage forms are known in the art, or will be apparent, to those skilled in this art; (for example, see REMINGTON: THE SCIENCE AND PRACTICE OF PHARMACY, 20.sup.th Ed., Gennaro, A. R. (ed.), Mack Publishing Company, Easton, Pa., 2000). The composition or formulation of the invention can contain a sufficient quantity of a calpain inhibitor to reduce calpain activity in vivo, thereby treating the inflammation-related disease or disroderstate in the subject.

Calpain inhibitors for use in the methods described herein for the treatment of an inflammation-related disease or disorder, can be administered as pharmaceutical compositions comprising an active therapeutic agent, i.e., and a variety of other pharmaceutically acceptable components (see, e.g., REMINGTON: THE SCIENCE AND PRACTICE OF PHARMACY, 20.sup.th Ed., Gennaro, A. R. (ed.), Mack Publishing Company, Easton, Pa., 2000, incorporated herein by reference). The form can depend on the intended mode of administration and therapeutic application. The compositions can also include, depending on the formulation, pharmaceutically-acceptable, non-toxic carriers or diluents, which are defined as vehicles used to formulate pharmaceutical compositions for animal or human administration. The diluent can be selected so as not to affect the biological activity of the combination. Examples of such diluents are distilled water, physiological phosphate-buffered saline, Ringer's solutions, dextrose solution, and Hank's solution. In addition, the pharmaceutical composition or formulation can also include other carriers, adjuvants, or nontoxic, nontherapeutic, nonimmunogenic stabilizers and the like.

Pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

Calcium Chelators

In one embodiment, calcium chelators can also be used in combination with any of the calpain inhibitors described herein. Any calcium chelator known in the art can be used in conjunction with the methods described herein. Exemplary calcium chelators suitable for use with the methods disclosed herein include, but are not limited to BAPTA tetrasodium salt, 5,5′-Dibromo-BAPTA tetrasodium salt, BAPTA/AM, 5,5′-Difluoro-BAPTA/AM, EDTA tetrasodium salt (Ethylenediamine tetraacetic acid), EGTA (Ethylenebis(oxyethylenenitrilo)tetraacetic acid), EGTA/AM, MAPTAM, and TPEN.

Calcium blockers are also suitable for use with the methods disclosed herein. Such compounds can include those that inhibit the release of calcium ions from intracellular calcium storage thereby blocking signaling through the Ca2+ levels. Exemplary calcium blockers suitable for use with the methods disclosed herein include, but are not limited to 1,4-dihydropyridine derivatives such as nifedipine, nicardipine, niludipine, nimodipine, nisoldipine, nitrendipine, milbadipine, dazodipine, and ferodipine; N-methyl-N-homoveratrilamine derivatives such as verapamil, gallopamil, and tiapamil; benzothiazepine derivatives such as diltiazem; piperazine derivatives such as cinnarizine, lidoflazine, and flunarizine; diphenylpropiramine derivatives such as prenylamine, terodiline, and phendiline; bepridil; and perhexyline.

Treatment Regimes

The invention provides calpain inhibitors and calpain inhibitors in pharmaceutical compositions formulated together with a pharmaceutically acceptable carrier. Some compositions can include a combination of multiple (e.g., two or more) small chemical molecules, siRNA molecules, monoclonal antibodies or antigen-binding portions thereof of the invention. In some compositions, each of the antibodies or antigen-binding portions thereof of the composition can be a monoclonal antibody or a human sequence antibody that binds to a distinct, pre-selected epitope of an antigen.

The pharmaceutical compositions of the invention can be administered in a variety of unit dosage forms depending upon the method of administration. Dosages for the pharmaceutical compositions of the invention can be readily determined by those of skill in the art. Such dosages can be adjusted depending on the therapeutic context, subject tolerance, and the like. The dosage schedule and amounts effective for this use, i.e., the dosing regimen, can depend upon a variety of factors, including the stage of the disease or condition, the severity of the disease or condition, the general state of the subject's health, the subject's physical status, age, pharmaceutical formulation and concentration of active agent, and the like. In calculating the dosage regimen for a subject, the mode of administration also can be taken into consideration. The dosage regimen can also take into consideration the pharmacokinetics, e.g., the pharmaceutical composition's rate of absorption, bioavailability, metabolism, clearance, and the like.

In therapeutic applications, compositions can be administered to a subject suffering from an inflammation-related disease or disorder to at least partially reduce the condition or a disease and/or its complications.

In prophylactic applications, pharmaceutical compositions or medicaments can be administered to a subject susceptible to, or otherwise at risk of an inflammation-related disease or disorder in an amount sufficient to eliminate or reduce the risk, lessen the severity, or delay the outset of the disease or disorder, including biochemical, histologic and/or behavioral symptoms of the disease, its complications and intermediate pathological phenotypes presenting during development of the disease. In therapeutic applications, compositions or can be administered to a subject suspected of, or already suffering from such a disease in an amount sufficient to cure, or at least partially reduce, the symptoms of the disease (biochemical, histologic and/or behavioral), including its complications and intermediate pathological phenotypes in development of the disease. In both prophylactic and therapeutic regimes, agents can be administered in several dosages until a level of inflammatory response has been achieved. The inflammatory response can be monitored and repeated dosages can be given if a further reduction in inflammation is needed.

In some applications, one or more additional compounds can be administered concomitantly with a calpain inhibitor. Such concomitant administration can involve concurrent (i.e., at the same time), prior, or subsequent administration of the drug with respect to the administration of a compound of the invention described herein. A person of ordinary skill in the art can readily determine the appropriate timing, sequence and dosages of administration for drugs and compounds use in the methods described herein.

Effective Dosages

Effective doses of calpain inhibitors for the treatment of inflammatory-related inflammation-related diseases or disorders with the methods described herein vary depending upon many different factors, including means of administration, target site, physiological state of the subject, whether the subject can be human or an animal, other medications administered, and whether treatment can be prophylactic or therapeutic. The subject can be a human but non-human mammals including transgenic mammals can also be treated with the methods described herein. Treatment dosages can be titrated to optimize safety and efficacy.

For administration with calpain inhibitor, for example a small chemical molecule, nucleic acid, siRNA, peptide, peptidomimetic, or antibody composition, the dosage can range according to the body weight of the subject to be administered with a calpain inhibitor. One skilled in the art can readily determine suitable dosage ranges. Calpain inhibitors can be administered on multiple occasions. Intervals between single dosages can be, hourly, daily, weekly, monthly or yearly. Intervals can also be irregular as indicated by measuring levels of one or more calpain inhibitors in the subject. Alternatively, calpain inhibitors disclosed herein can be administered as a sustained release formulation, in which case less frequent administration can be required. Dosage and frequency vary depending on the half-life of the calpain inhibitor in the subject. The dosage and frequency of administration can vary depending on whether the treatment is prophylactic or therapeutic. In prophylactic applications, a relatively low dosage can be administered at relatively infrequent intervals over a long period of time. In some subjects administration of calpain inhibitors can be continued for the rest of the subject's life. In therapeutic applications, a relatively high dosage at relatively short intervals can sometimes be required until progression of the inflammatory disorder is reduced or terminated. Thereafter, the subject can be administered a prophylactic regime.

Dosage forms or compositions can optionally comprise one or more calpain inhibitors according to the invention described herein in the range of 0.0001% to 100% (weight/weight) with the balance comprising additional substances, excipient or aduvants such as those described herein. For oral administration, a pharmaceutically acceptable composition can optionally comprise any one or more employed excipients, such as, for example pharmaceutical grades of mannitol, lactose, starch, magnesium stearate, talcum, cellulose derivatives, sodium crosscarmellose, glucose, sucrose, magnesium carbonate, sodium saccharin, talcum. Such compositions include solutions, suspensions, tablets, capsules, powders, dry powders for inhalers and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparing these formulations are known to those skilled in the art.

Salts, or sodium salts, of the calpain inhibitors can be prepared with carriers that protect the compound against rapid elimination from the body, such as time release formulations or coatings. The formulations can further include other active compounds to obtain combinations of properties.

Prodrugs

The invention described herein is also related to prodrugs of the agents used in the methods disclosed herein. Prodrugs are agents which are converted in vivo to active forms (see, e.g., R. B. Silverman, 1992, THE ORGANIC CHEMISTRY OF DRUG DESIGN AND DRUG ACTION, Academic Press, Chp. 8). Prodrugs can be used to alter the biodistribution (e.g., to allow agents which do not enter the reactive site of the protease) or the pharmacokinetics for an agent. For example, a carboxylic acid group, can be esterified, e.g., with a methyl group or an ethyl group to yield an ester. When the ester is administered to a subject, the ester is cleaved, enzymatically or non-enzymatically, reductively, oxidatively, or hydrolytically, to reveal the anionic group. An anionic group can be esterified with moieties (e.g., acyloxymethyl esters) which are cleaved to reveal an intermediate agent which subsequently decomposes to yield the active agent. The prodrug moieties can be metabolized in vivo by esterases or by other mechanisms to carboxylic acids.

Examples of prodrugs and their uses are well known in the art (see, e.g., Berge et al., “Pharmaceutical Salts”, J. Pharm. Sci. 66: 1-19, 1977). The prodrugs can be prepared in situ during the final isolation and purification of the agents, or by separately reacting the purified agent in its free acid form with a suitable derivatizing agent. Carboxylic acids can be converted into esters via treatment with an alcohol in the presence of a catalyst.

Examples of cleavable carboxylic acid prodrug moieties include substituted and unsubstituted, branched or unbranched lower alkyl ester moieties, (e.g., ethyl esters, propyl esters, butyl esters, pentyl esters, cyclopentyl esters, hexyl esters, cyclohexyl esters), lower alkenyl esters, dilower alkyl-amino lower-alkyl esters (e.g., dimethylaminoethyl ester), acylamino lower alkyl esters, acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, dilower alkyl amides, and hydroxy amides.

Toxicity

A therapeutically effective dose of the calpain inhibitors for use with the methods described herein can be provided for therapeutic without causing substantial toxicity. Toxicity of the calpain inhibitors for use with the methods described herein can be determined by standard pharmaceutical procedures in cell cultures or experimental animals, e.g., by determining the LD.sub.50 (the dose lethal to 50% of the population) or the LD.sub.100 (the dose lethal to 100% of the population). The dose ratio between toxic and therapeutic effect is the therapeutic index. The data obtained from these cell culture assays and animal studies can be used in formulating a dosage range that is not toxic for use in a human. The dosage of the proteins described herein can lie within a range of circulating concentrations that include the effective dose with little or no toxicity. The dosage can vary within this range depending upon the dosage form employed and the route of administration utilized. The exact formulation, route of administration and dosage can be chosen by the individual physician in view of the subject's condition (see, e.g., Fingl et al., 1975, In: THE PHARMACOLOGICAL BASIS OF THERAPEUTICS, Ch. 1 pl).

Kits

The invention is also directed to kits and other articles of manufacture for treating inflammation-related diseases and disorders. In one aspect, a kit is provided that comprises a composition comprising at least one calpain inhibitor for use in the methods described herein in combination with instructions. The instructions can indicate the inflammation-related diseases and disorder for which the composition is to be administered, storage information, dosing information and/or instructions regarding how to administer the composition. The kit can also comprise packaging materials. The packaging material can comprise a container for housing the composition. The kit can also optionally comprise additional components, such as syringes for administration of the composition. The kit can comprise the composition in single or multiple dose forms.

In another aspect, an article of manufacture can be provided that comprises a composition for use in conjunction with the methods described herein comprising at least one calpain inhibitor described herein in combination with packaging materials. The packaging material can comprise a container for housing the composition. The container can optionally comprise a label indicating the disease state for which the composition is to be administered, storage information, dosing information and/or instructions regarding how to administer the composition. The kit can also optionally comprise additional components, such as syringes for administration of the composition. The kit can comprise the composition in single or multiple dose forms.

Packaging material used in kits and articles of manufacture according to the invention described herein can form a plurality of divided containers such as a divided bottle or a divided foil packet. The container can be in any conventional shape or form as known in the art which is made of a pharmaceutically acceptable material, for example a paper or cardboard box, a glass or plastic bottle or jar, a re-sealable bag (for example, to hold a refill of tablets for placement into a different container), or a blister pack with individual doses for pressing out of the pack according to a therapeutic schedule. The container that is employed can depend on the exact dosage form involved. More than one container can be used together in a single package to market a single dosage form. For example, tablets can be contained in a bottle that is in turn contained within a box. The kit can include directions for the administration of the separate components. The kit form can be advantageous when the separate components can be administered in different dosage forms (e.g., for inhalation, oral, topical, transdermal and parenteral administration), are administered at different dosage intervals, or when the prescribing physician determines that titration of the individual components of the combination is important.

In one embodiment, the kit can be a dispenser designed to dispense the daily doses one at a time in the order of their intended use. The dispenser can be equipped with a memory-aid, so as to further facilitate compliance with the regimen. An example of such a memory-aid is a mechanical counter that indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

Screening Assays

As discussed herein, it can be important to inhibit or reduce calpain activity. In one aspect, the invention provides a target for the screening of libraries and for designing and identifying potent and selective inhibitors or modulators of calpain proteins useful in the treatment of inflammation or inflammation associated conditions.

This invention provides screening methods useful for identifying modulators, that is, candidate or test compounds or agents (e.g., polypeptides, peptidomimetics, small molecules, or other drugs) which bind to calpain or to polypeptides of this invention, have a stimulatory or inhibitory effect on, for example, calpain activity.

High-throughput screening of compound libraries to identify molecules having a desired molecular interaction/effect is one approach for drug discovery and test compound optimization. The test compounds can be obtained using any of the numerous approaches in combinatorial library methods known in the art, including: biological libraries; spatially addressable parallel solid phase or solution phase libraries; synthetic library methods requiring deconvolution; the one-bead one-compound library method; and synthetic library methods using affinity chromatography selection. The biological library approach is limited to polypeptide libraries, while the other four approaches are applicable to polypeptide, non-peptide oligomer, or small molecule libraries of compounds (Lam K S, Anticancer Drug Des. 12:145-67 (1997)). Such compound libraries are also available from commercial sources such as ComGenex (U.S. Headquarters, South San Francisco, Calif.), Maybridge (Cornwall, UK), and SPECS (Rijswijk, Netherlands).

Compound screening or assay development can be performed on semi-automated workstations or on fully-automated robots, such as the Tecan Genesis 200 platform. Assays can be developed for a variety of 96/384 well liquid handling equipment capable of both normal or low volume assay formats. In the design of new assays for drug discovery screening, fluorescence-based detection technologies can be suited to high-throughput applications.

Such technologies can be applied to the identification of compounds that bind to calpain or to the identification of compounds that inhibits calpain cleavage of occludin, E-cadherin or Ezrin or of any fragments thereof as described herein. The identification of such a compound can be accomplished by introducing test compounds from a compound library, independently, into an incubation mixture containing: 1) one or more calpain substrates described herein, for example Occludin, E-cadherin, Ezrin, the N-terminal intracellular domain of Occludin and the like, 2) calpain and 3) a test compound and 4) measuring cleavage of the substrate by calpain. Any method of measuring calpain activity known in the art can be used in conjunction with the screening methods described herein.

In one embodiment, a compound can be tested for the ability to inhibit calpain cleavage of a polypeptide having the sequence shown in any of SEQ ID NOS: 44 or 45. In another embodiment, a compound can be tested for the ability to inhibit calpain cleavage of the 80 kDa hyperphosphorylated form of occludin. In another embodiment, a compound can be tested for the ability to inhibit calpain cleavage of E-Cadherin. In another embodiment, a compound can be tested for the ability to inhibit calpain cleavage of Ezrin. In still a further embodiment, the calpain substrate can be linked to a detectable marker (for example an, epitope tag or a GFP polypeptide) to facilitate detection of substrate cleavage. Any method for detecting cleavage of a substrate known in the art is suitable with the screening methods disclosed herein.

Examples of such compounds include, but are not limited to, small organic molecules including pharmaceutically acceptable molecules. Examples of small molecules include, but are not limited to, polypeptides, peptidomimetics, amino acids, amino acid analogs, nucleic acids, nucleic acid analogs, nucleotides, nucleotide analogs, organic or inorganic compounds (i.e., including heteroorganic and organometallic compounds) generally having a molecular weight of less than 10,000 grams per mole, salts, esters, and other pharmaceutically acceptable forms of such compounds. Examples of other compounds that can be tested in the methods of this invention include polypeptides, antibodies, nucleic acids, and nucleic acid analogs, natural products and carbohydrates.

Compounds for use in the methods of this invention can be obtained using any of the numerous approaches in combinatorial methods know in the art including: biological libraries; spatially addressable parallel solid phase or solution phase libraries; synthetic library methods requiring deconvolution; the one-bead one-compound library method; and synthetic library methods using affinity chromatography selection. Many organizations (e.g., the National Institutes of Health, pharmaceutical and chemical corporations) have large libraries of chemical or biological compounds from natural or synthetic processes, or fermentation broths or extracts.

A compound can have a known chemical structure but not necessarily have a known function or biological activity. Compounds can also have unidentified structures or be mixtures of unknown compounds, for example from crude biological samples such as plant extracts. Large numbers of compounds can be randomly screened from chemical libraries, or collections of purified chemical compounds. or collections of crude extracts from various sources. The chemical libraries can contain compounds that were chemically synthesized or purified from natural products. The compounds can comprise inorganic or organic small molecules or larger organic compounds such as, for example, proteins, polypeptides, steroids, lipids, phospholipids, nucleic acids, and lipoproteins. The amount of compound tested can vary depending on the chemical library. Methods of introducing test compounds to cells are well known in the art.

Examples of methods for the synthesis of molecular libraries can be found in the art, for example in: DeWitt S H et al., Proc. Natl. Acad. Sci. USA 90:6909-13 (1993); Erb E et al., Proc. Natl. Acad. Sci. USA 91:11422-26 (1994); Zuckermann R N et al., J. Med. Chem. 37:2678-85 (1994); Cho C Yet al., Science 261:1303-05 (1993); Carrell T et al., Angew. Chem. Int. Ed. Engl. 33:2059-61 (1994); Carrell T et al., Angew. Chem. Int. Ed. Engl. 33:2061-64 (1994); and Gallop M A et al., J. Med. Chem. 37:1233-51 (1994).

Libraries of compounds can be presented, for example, in solution (e.g., Houghten R A et al., Biotechniques 13:412-21 (1992)), or on beads (Lam K S et al., Nature 354:82-84 (1991)), chips (Fodor S P A et al., Nature 364:555-56 (1993)), bacteria (U.S. Pat. No. 5,223,409), spores (U.S. Pat. No. 5,223,409), plasmids (Cull M G et al., Proc. Natl. Acad. Sci. USA 89:1865-69 (1992)), or on phage (Scott J K and Smith G P, Science 249:386-90 (1990); Devlin J J et al., Science 249:404-06 (1990); Cwirla S E et al., Proc. Natl. Acad. Sci. 87:6378-82 (1990); Felici F et al., J. Mol. Biol. 222:301-10 (1991); U.S. Pat. No. 5,223,409).

In many drug screening programs which test libraries of modulating agents and natural extracts, high throughput assays are desirable in order to maximize the number of modulating agents surveyed in a given period of time. Assays which are performed in cell-free systems, such as can be derived with purified or semi-purified proteins, are often used as primary screens because they can be generated to permit rapid development and relatively easy detection of an alteration in a molecular target which is mediated by a test modulating agent. Moreover, the effects of cellular toxicity and/or bioavailability of the test modulating agent can be generally ignored in the in vitro system, the assay instead being focused primarily on the effect of the drug on the molecular target as can be manifest in an alteration of binding affinity with upstream or downstream elements.

It will be readily apparent to those skilled in the relevant arts that other suitable modifications and adaptations to the methods and applications described herein can be made without departing from the scope of the invention or any embodiment thereof.

The following examples illustrate the invention described herein, and are set forth to aid in the understanding of the invention, and should not be construed to limit in any way the scope of the invention as defined in the claims which follow thereafter.

The following methods can be used in connection with the embodiments of the invention.

### EXAMPLES

### Example 1

### Bacterial Activation of TLR2 Modulates Epithelial Barrier Function

Bacterial stimulation of TLR2 signaling initiates chemokine expression in epithelial cells which is followed by the recruitment of phagocytes into the airway lumen. This process can be linked to changes in the epithelial cell-cell junctions to facilitate the movement of phagocytes into the airway without compromising the barrier function of the epithelium. TLR2 activation can be accompanied by the activation of Ca²⁺ fluxes in epithelial cells and Ca²⁺-dependent proteases, such as a calpain, can be involved in modifying epithelial junctional proteins in response to TLR2-specific ligands. Using 1HAEo-human airway cell lines as well as human small airway epithelial cells in primary culture, P. aeruginosa or the TLR2 agonist Pam3Cys activates calpain 2, was shown cause cleavage of the transmembrane proteins E-cadherin and occludin. Calpain 2 is recruited to the epithelial membrane in response to TLR2 ligands and is associated with both E-cadherin and occludin by 1 hour following stimulation, as demonstrated by immunoprecipitation and confocal imaging. Calpain activity is both TLR2 and Ca²⁺ dependent. Cleavage of a fluorescent calpain substrate, as well as the generation of occludin cleavage products, is inhibited in cells expressing TLR2 siRNA, TLR2YY mutations or in the presence of calpain inhibitors. These changes in junctional proteins did not alter transepithelial resistance across monolayers but did alter permeability to fluorescent dextrans. The results described herein show that TLR2 signaling affects the barrier properties of the airway epithelium through a Ca²⁺ dependent signaling cascade.

Airway epithelial cells are a component of inflammatory responses of the mucosal immune system. These cells provide both signaling and barrier functions to protect the lungs from inhaled pathogens. Toll-Like Receptor 2 (TLR2) is displayed on the exposed, apical surface of airway epithelial cells and is broadly responsive to pathogen associated molecular patterns (PAMPS) from clinically important respiratory pathogens, including the Gram negative opportunist Pseudomonas aeruginosa. TLR2 signaling results in epithelial production of the cytokine granulocyte-macrophage colony-stimulating factor (GM-CSF) and the chemokine interleukin-8 (IL-8), which are important in the recruitment and activation of polymorphonuclear leukocytes (PMNs). For these phagocytes in the bloodstream to reach bacteria in the airway lumen, they must migrate across both the endothelial barrier and the epithelial barrier, which are normally tightly apposed via adherens and tight junction (TJ) proteins (Wagner and Roth, 2000). The cascade of adhesive, stimulatory, and guidance factors involved in PMN extravasation across endothelial cells and subsequent migration within the interstitial space has been studied in some detail (Burns et al., 2003). In comparison, less is known about the transepithelial migration of PMNs. Since the major function of the epithelial TJs is to create a barrier to maintain sterility in the lung, they are more substantial and ten times less leaky than endothelial junctions (Burns et al., 2003). Disruption of the TJ in the presence of infection in the airway lumen can facilitate bacterial invasion and disseminate infection. The ability of PMNs are able to squeeze between respiratory epithelial cells without breaching the barrier provided by TJs is important in controlling inflammation in the airway, which, if excessive, blocks air exchange and causes respiratory failure.

Tight junctions (TJ) are maintained through a complex network of interacting proteins of several different types (Harhal et al, 2004). Some of the junctional complex proteins, such as occludin and E-cadherin, span the plasma membrane and associate through homotypic interactions with corresponding domains on adjacent cells (Feldman et al, 2005; Mege et al, 2006). Occludin, one of the first TJ proteins to be identified has two extracellular loops linking adjacent cells. These extracellular domains affect PMN migration between endothelial cells (Feldman et al, 2006). Occludin can undergo endocytosis through a caveolin-1 mediated processes in response to various signals such as Mitogen Activated Kinases (MAPKs) and oxidative (Shen et al, 2005). Although not essential to maintain the tight junction (Saitou et al, 2000; Schulzke et al, 2005), occludin has an important regulatory function, in that it interacts with several other junctional proteins including ZO-1 and indirectly actin and the cytoskeleton (Muller et al, 2005).

E-cadherin is also important for maintenance of the epithelial barrier and spans the paracellular space through five extracellular domains (Bryant et al, 2004). E-cadherin can undergo endocytosis in response to various stimuli as part of the dynamic process of membrane homeostasis (Mege et al, 2006; Bryant et al, 2004). The distribution of these junctional proteins can be altered to facilitate phagocyte recruitment across the epithelium as part of the epithelial response to bacterial stimuli. Accumulation of PMNs into the airway lumen occurs following bacterial exposure (Wagner et al, 2000; Reutershan et al, 2005) demonstrating that changes in the permeability characteristics of the paracellular junctions can be an immediate consequence of the epithelial proinflammatory signaling cascade.

TLR2 functions as a major epithelial receptor responding to components of bacterial pathogens in the airway lumen (Adamo et al, 2004; Soong et al, 2004; Fournier et al, 2005). In response to ligands, TLR2 is actively mobilized to the apical surface of the airway cell in response to ligands where it can be phosphorylated by c-Src and recruit PI3K and PLCγ thereby releasing Ca²⁺ from intracellular stores and stimulating MAPK activity and NF-κB translocation to initiate chemokine and cytokine expression (Chun et al, 2006). The signaling cascade that initiates chemokine expression can also include a mechanism to facilitate PMN mobilization across the epithelial barrier. In endothelial cells, Ca2+ signaling was shown to increase transendothelial migration of PMNs by opening their intercellular junctions (Huang et al., 1993). The Ca2+ flux was not required for PMN adhesion, but was important for PMN migration across human umbilical vein endothelial cell (HUVEC) monolayers (Huang et al., 1993). The exact mechanism of Ca2+-dependent PMN migration, however, remains to be examined. As Ca²⁺ often functions as a second messenger, Ca²⁺ dependent proteases can be involved in modulating junctional proteins to accommodate PMN transmigration to the airway lumen.

Calpain 1 (mu-calpain) and calpain 2 (m-calpain) are Ca²⁺ dependent cysteine proteases that target cytoskeletal proteins in a number of cell types including the lung. They are known to be involved in cellular motility, apoptosis and inflammation (Goll et al, 2003). The results described herein show that Ca²⁺ fluxes initiated by TLR2 signaling activate calpains, which cleave both occludin and E-cadherin to increase recruitment of PMNs into the airway.

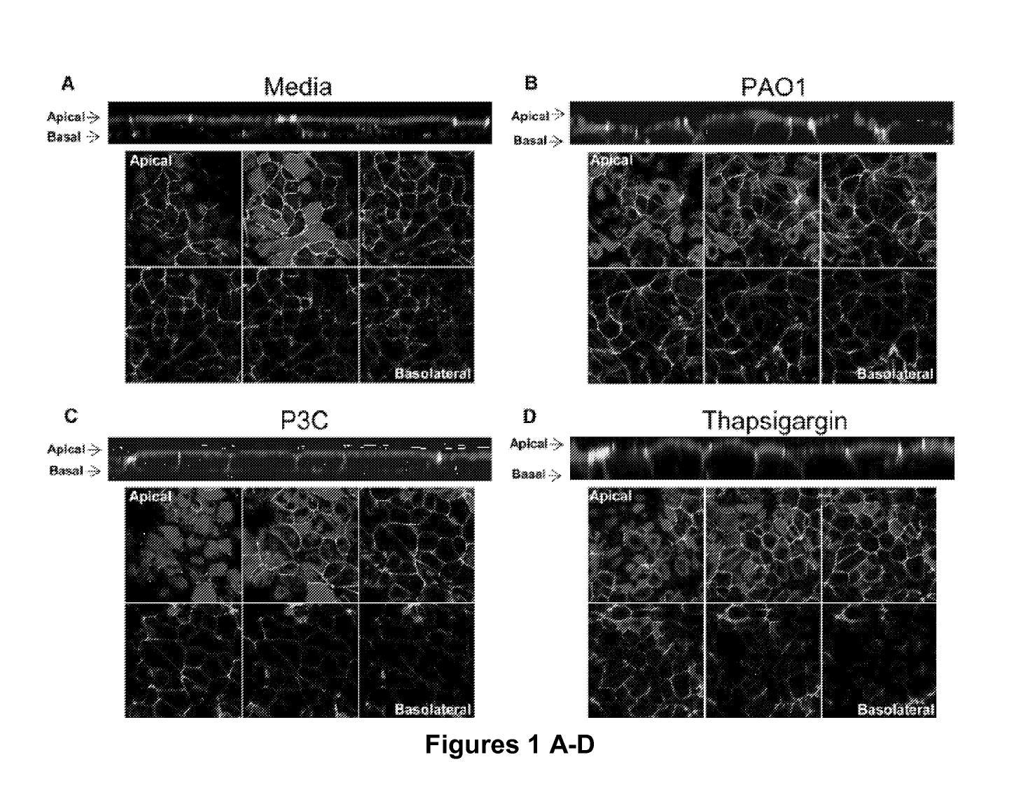

Organisms that cause pneumonia (e.g., opportunists such as P. aeruginosa) can gain access to the lung by inhalation and be encountered by the mucosal epithelium. To determine how the initial exposure to bacteria affects the tightness of the epithelial barrier, biotin detected with Alexa Fluor 555 conjugated streptavidin was used to outline the exposed surfaces of polarized airway epithelial monolayers. The ability of Alexa Fluor 555 conjugated to strepavidin to intercalate into the paracellular spaces was determined upon bacterial challenge or exposure to a TLR2 agonist (FIG. 1). In control monolayers, biotin was limited to the most apical surfaces of the monolayer and failed to intercalate between cells. In contrast, exposure to heat killed P. aeruginosa PAO1, to Pam₃Cys-Ser-Lys₄ (P3C) (a TLR2 agonist), or to thapsigargin (a sarcoplasmic/endoplasmic reticulum Ca²⁺ ATPase (SERCA) pump inhibitor (that stimulates release of Ca²⁺ from intracellular stores) resulted in the penetration of biotin (red) between the (red) between the cells in a polarized monolayer, both in z-sections and in images obtained from planes below the top of the monolayers. (FIG. 1).

### Example 2

### P. aeruginosa Stimulates the Redistribution of Occludin and E-Cadherin Without Loss of Barrier Function

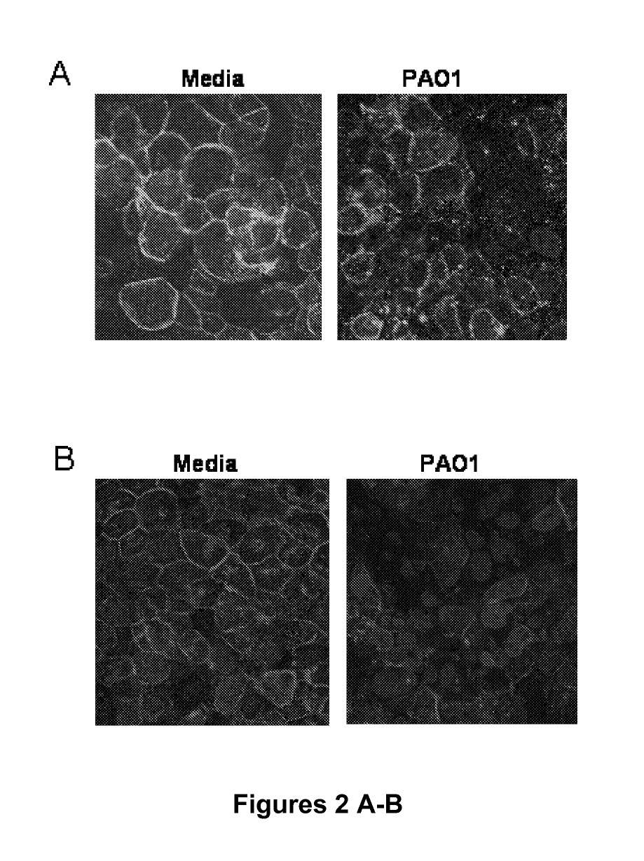

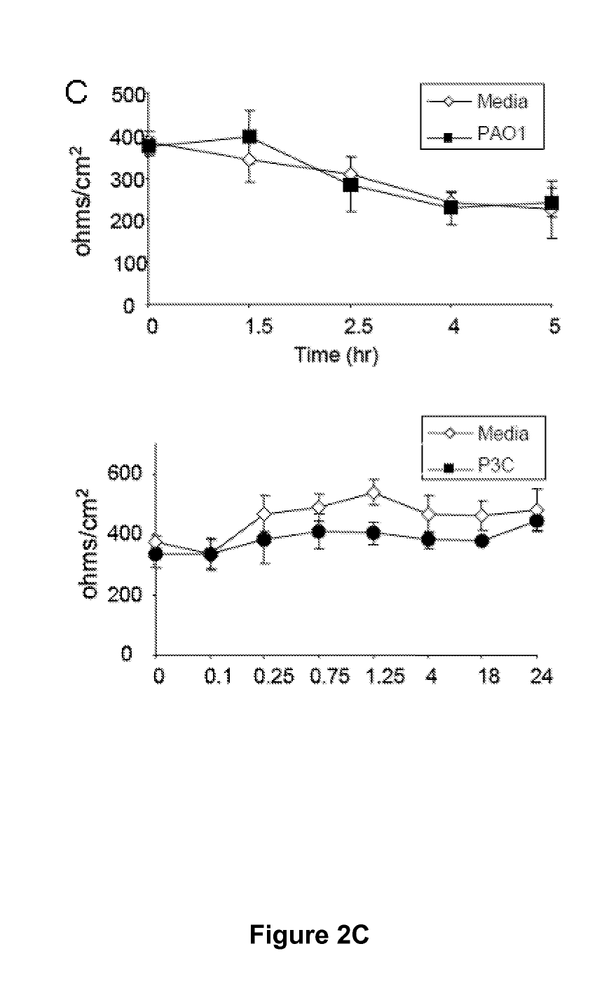

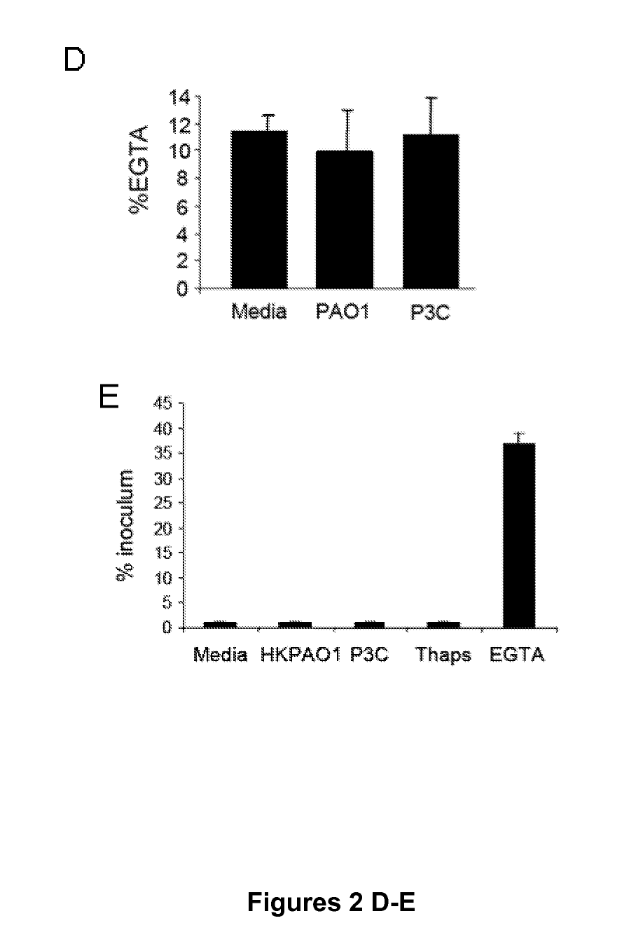

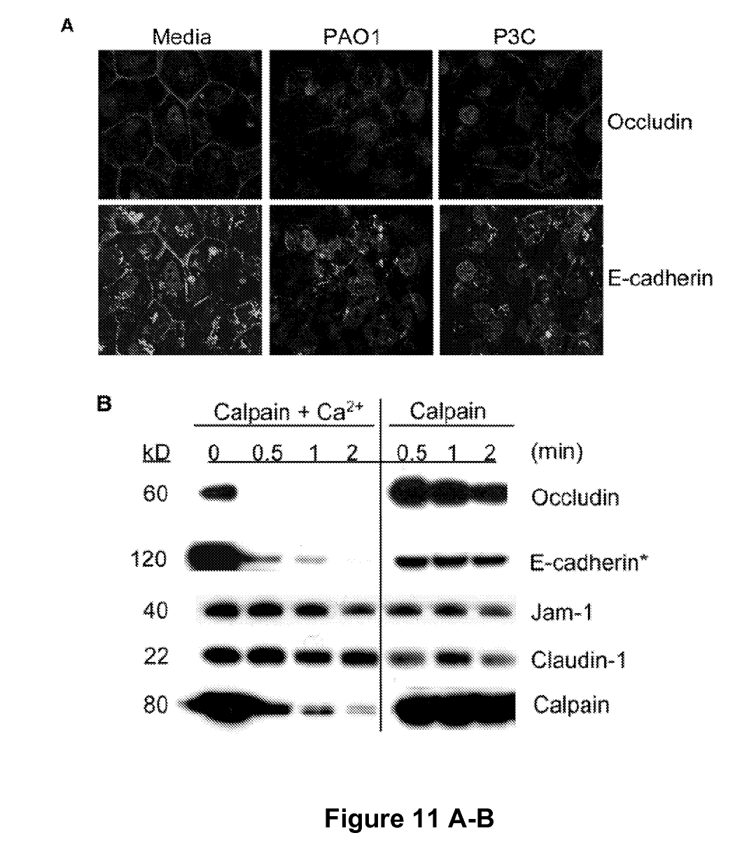

To demonstrate that TLR2 or Ca²⁺ signals induce changes in epithelial junctions, the distribution of the membrane-spanning junctional proteins occludin and E-cadherin were imaged. The “chicken wire” distribution of both occludin (FIG. 2A) and E-cadherin (FIG. 2B and FIG. 11B) was altered following 6 hr exposure of airway cells to P. aeruginosa PAO1 or PC3. Despite loss of occludin and E-cadherin at the cell borders, there was no concomitant decrease in the transepithelial resistance measured across the monolayers over this time period (FIG. 2C); nor was there an increase in permeability to fluorescent dextran (FIG. 2E), or to bacteria across the paracellular space (FIG. 2E) indicating that the barrier function of the monolayer remained intact (FIG. 2F).

### Example 3

### Occludin and E-Cadherin are Targets for Calpain Proteolysis

Occludin and E-cadherin are both substrates for proteases known to be responsible for their dissociation from the cell junctions (Bojarski et al., 2004; Rios-Doria et al., 2003; Zhu et al., 2006). Both occludin and E-cadherin are known substrates for proteases (Bojarski et al, 2004; Rios-Doria et al, 2003; Zhu et al, 2006). Among the many cellular proteases that can target these proteins, Ca²⁺-dependent calpains were examined to determine if TLR2 induced Ca²⁺-flux can initiate activity (Shao et al, 2006). In an in vitro experiment, exogenous calpain activated by the addition of Ca2+ degraded occludin and E-cadherin, but not claudin-1 or JAM-1 (FIG. 11B), indicating that calpains target specific junctional proteins. Autolysis of calpain is readily detectable in the presence of Ca2+ and confirms calpain activation in vitro (FIG. 11B).

Calpain is Activated by TLR2 Signaling

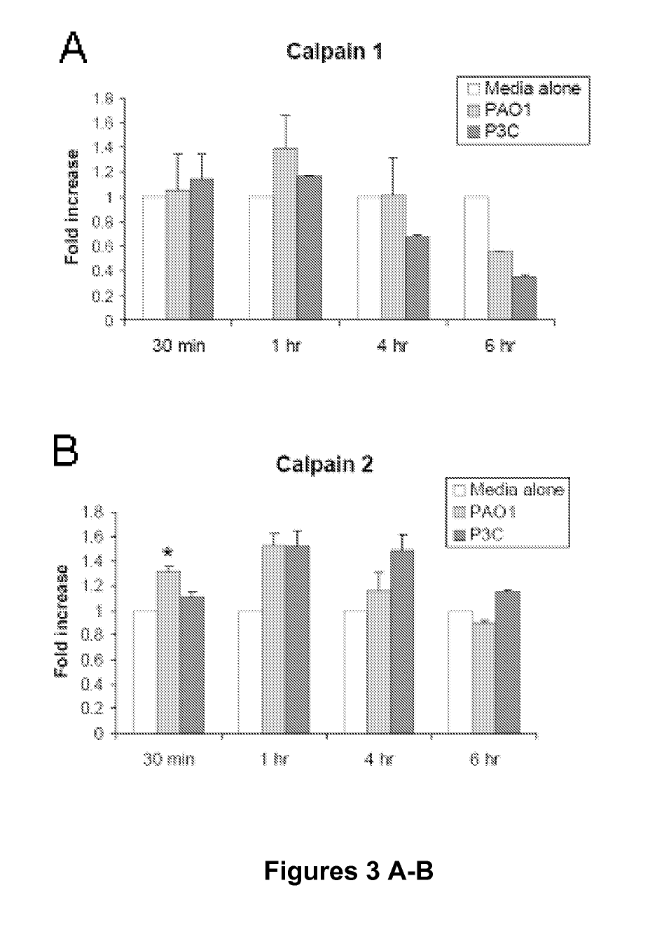

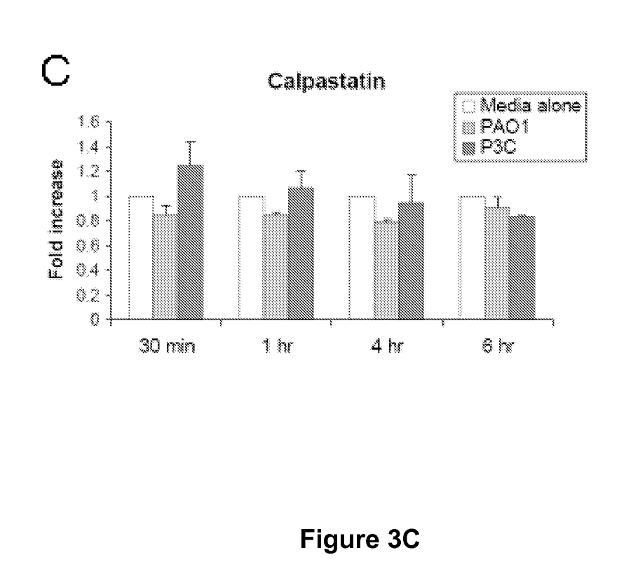

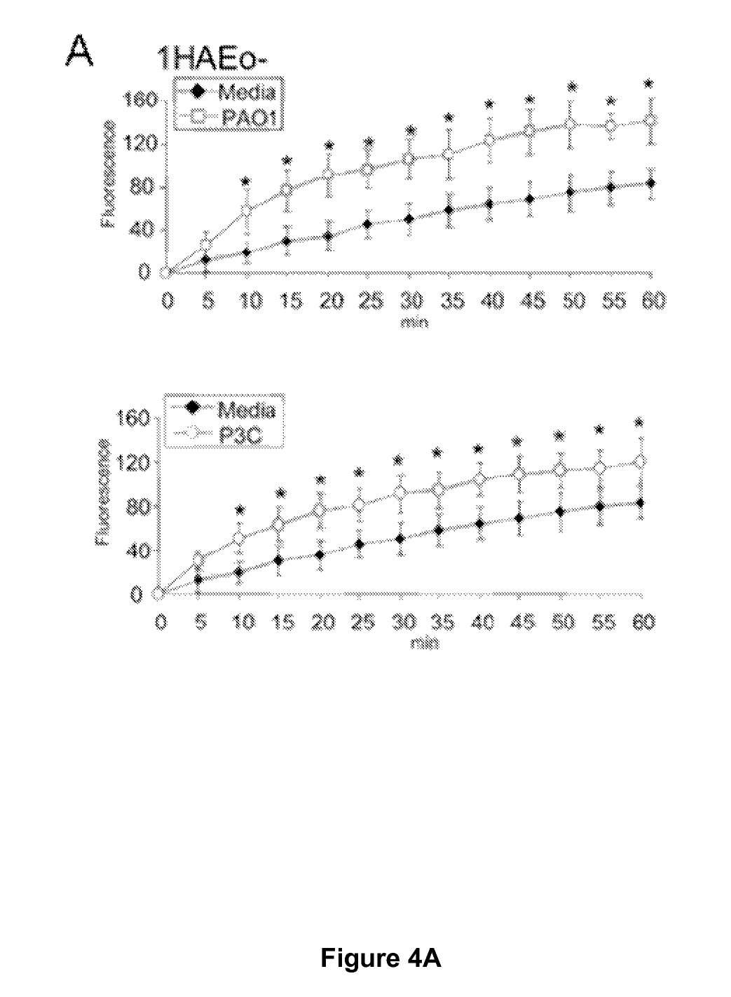

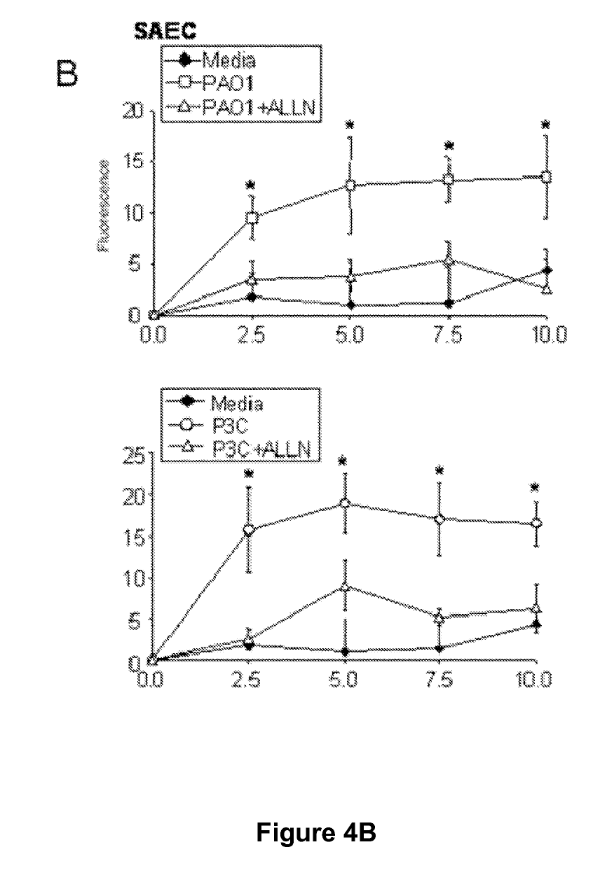

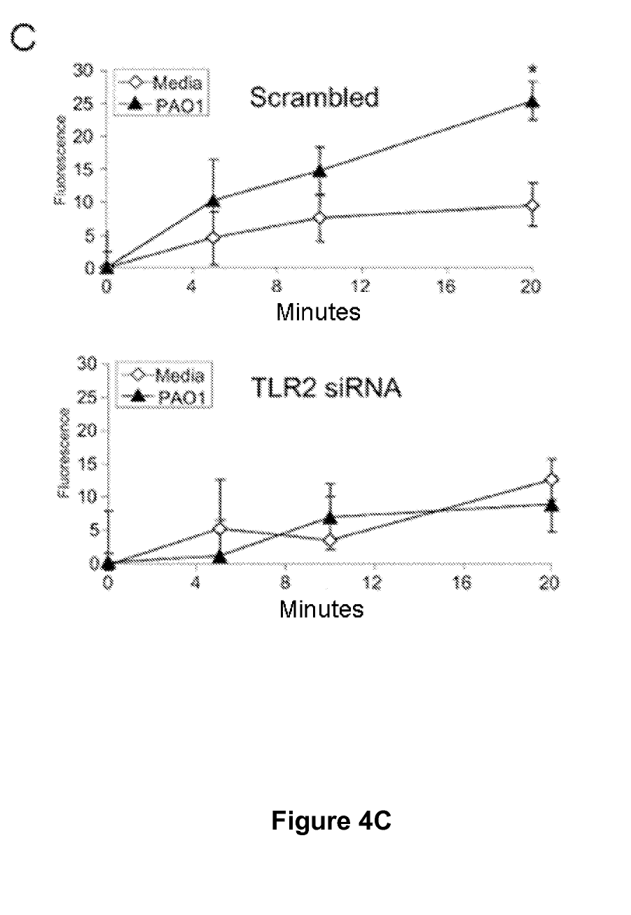

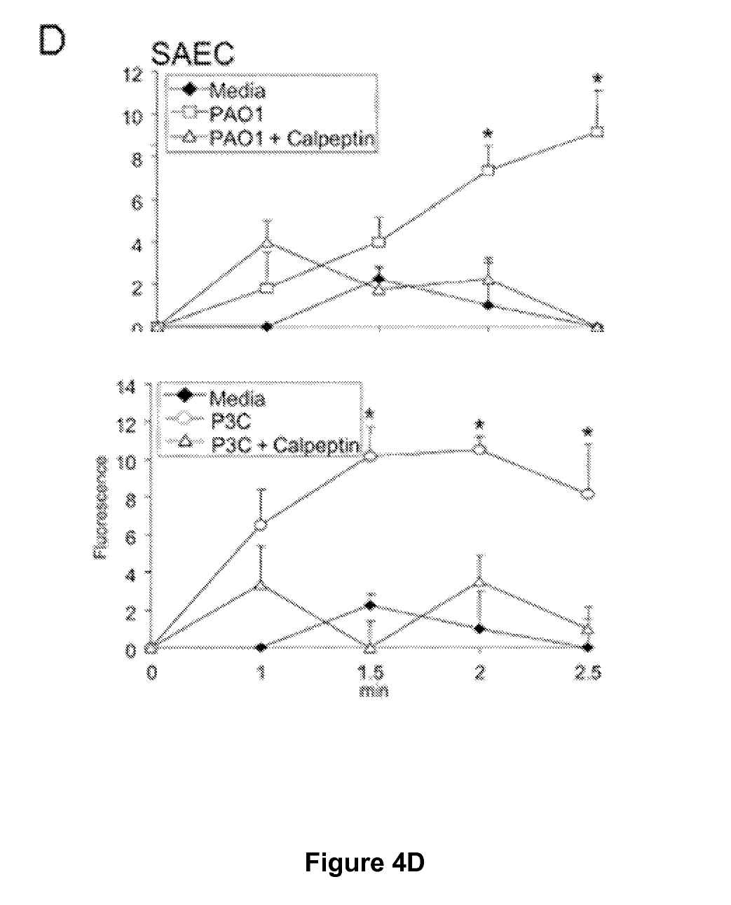

Calpains 1 and 2 as well as the endogenous inhibitor calpastatin are present in human airway cells and transcription of calpain 2 is increased following epithelial stimulation by bacteria or P3C (FIG. 3). Calpain activity in 1HAEo-cells and in human small airway cells in primary culture (SAEC), monitored by measuring accumulation of a cell permeable fluorogenic calpain substrate, was significantly increased following bacterial or P3C exposure (FIG. 4A) and inhibited in the presence of ALLN, a selective calpain inhibitor (FIG. 4B) and calpain, also a selective calpain inhibitor (FIG. 4D) Calpain activity was dependent upon TLR2 signaling. Calpain activity was not increased by bacterial stimulation of cells expressing TLR2 siRNA, as compared with a scrambled siRNA control (FIG. 4C). The TLR2 ligand, P3C, by itself was sufficient to activate calpain and the presence of its receptor was necessary for bacterial activation of the protease.

### Example 4

### Calpain Mediated Cleavage of Occludin is Activated by Bacterial Ligands

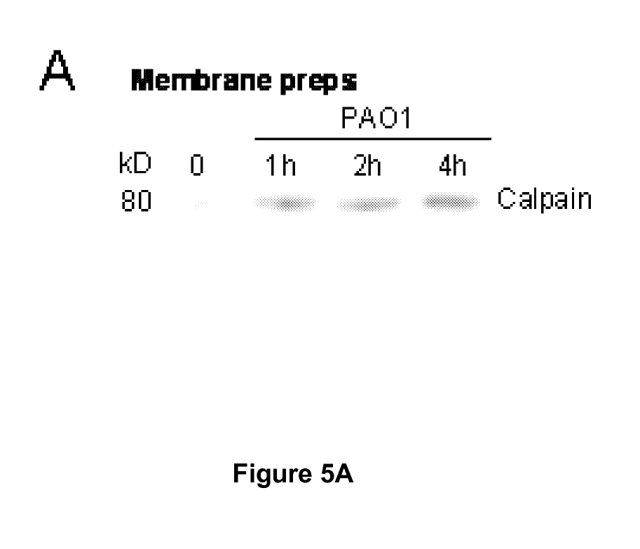

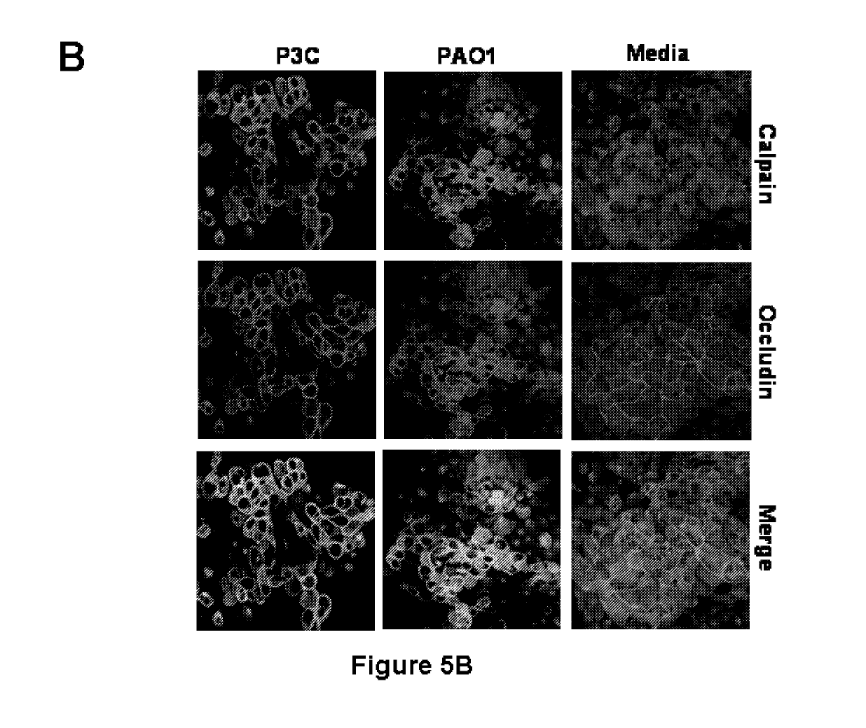

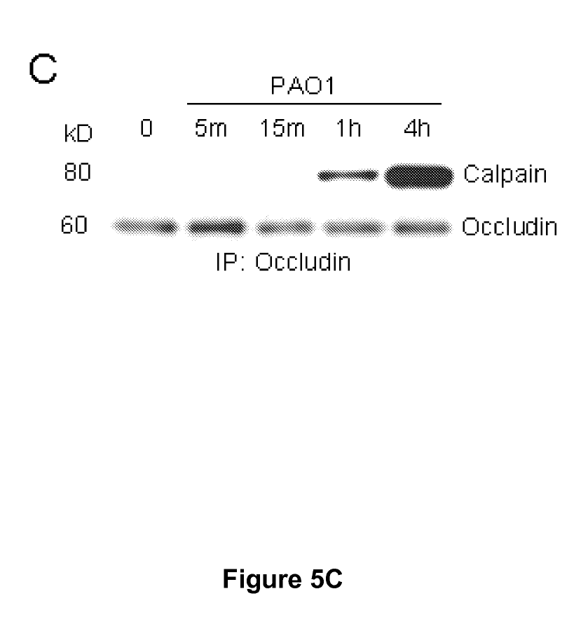

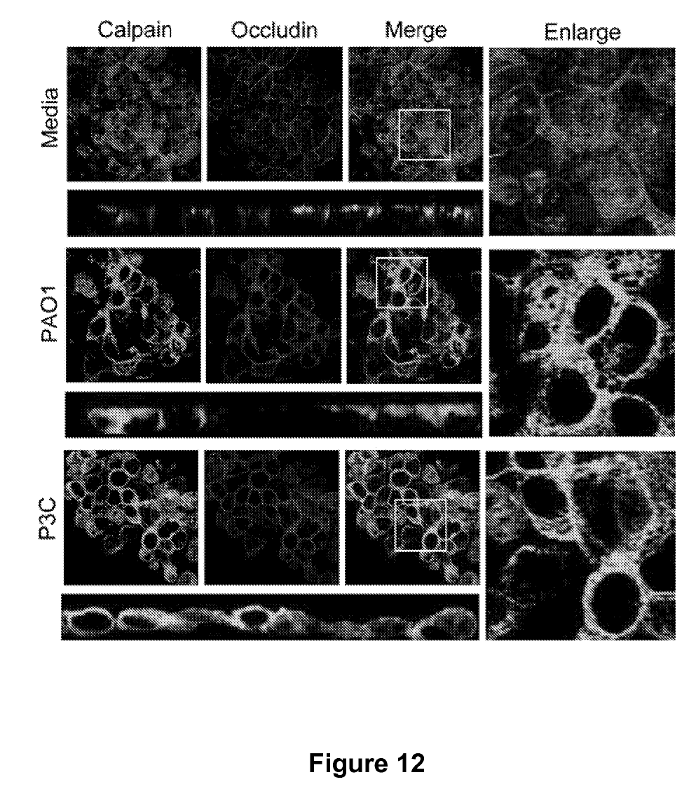

Given that bacterial stimulation mediated by TLR2 induces both the redistribution of occludin and the activation of calpain, occludin was examined to determine if it is a calpain substrate in airway cells (FIG. 5A). Calpain was found throughout the cytoplasm in confocal images of unstimulated airway cells, whereas occludin was concentrated at the plasma membrane (FIG. 5B and FIG. 12). Visualization of confocal images (FIG. 5B) and co-immunoprecipitation experiments (FIG. 5C) showed that calpain assumed a peripheral distribution and co-localized with membrane-associated occludin by one hour after bacterial or P3C exposure.

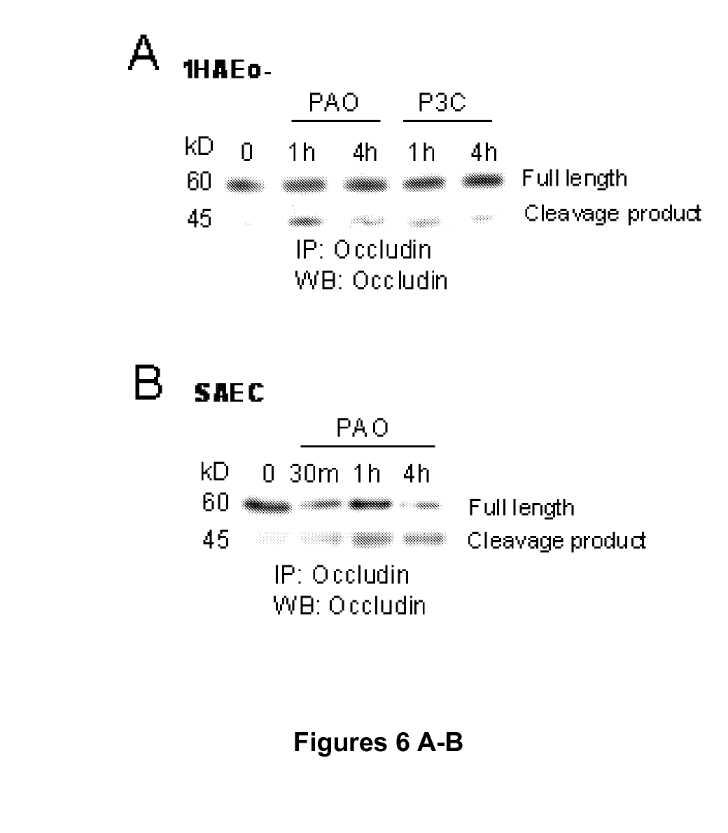

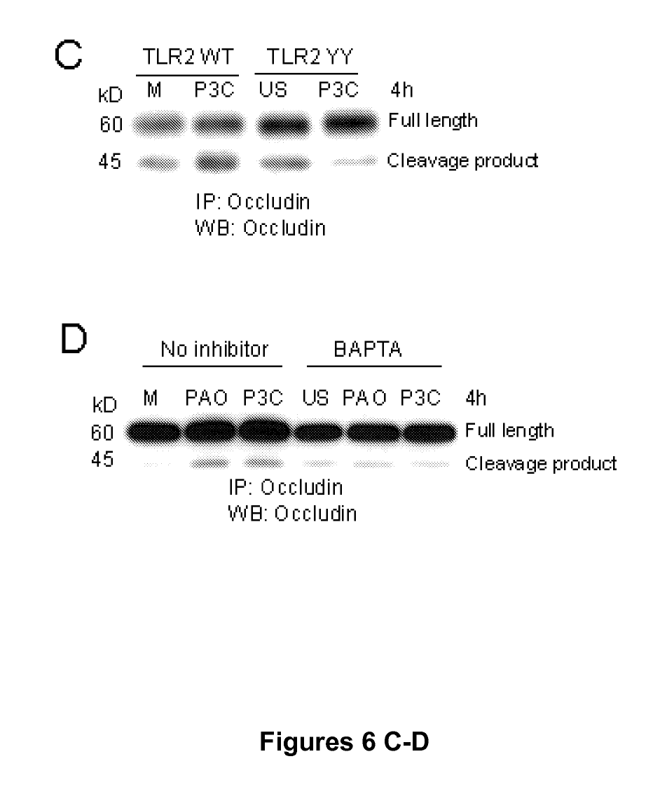

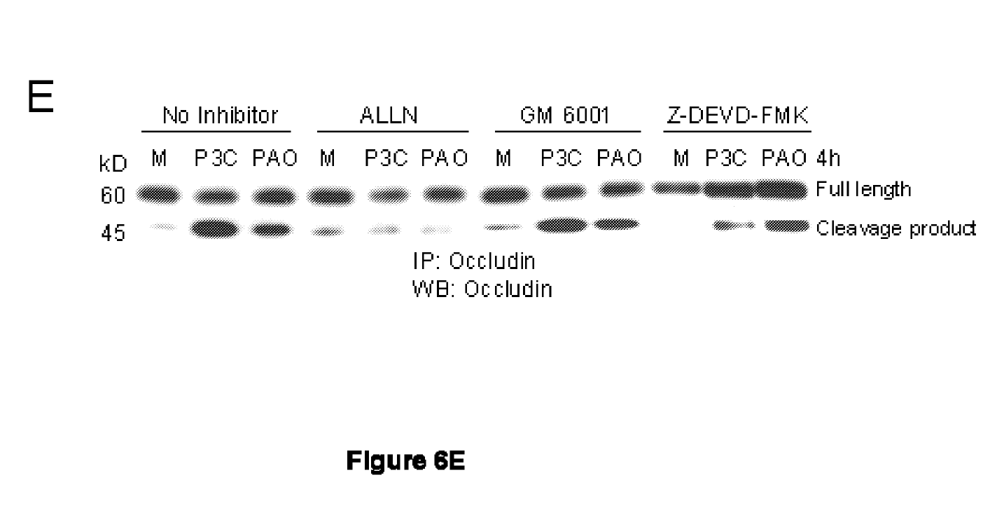

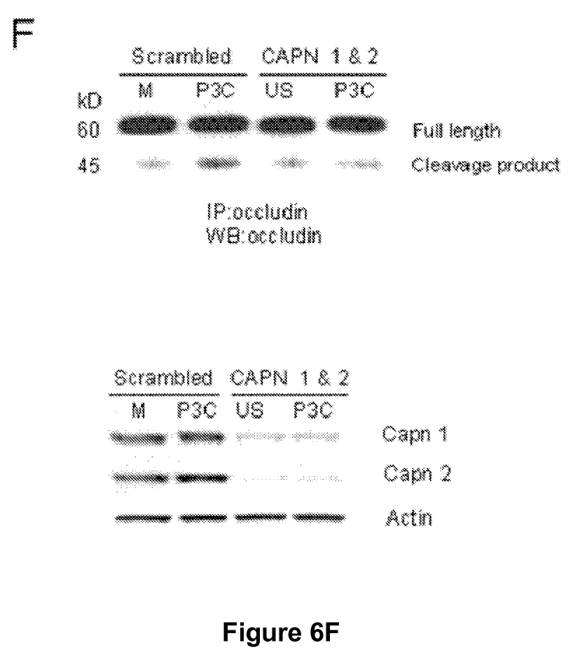

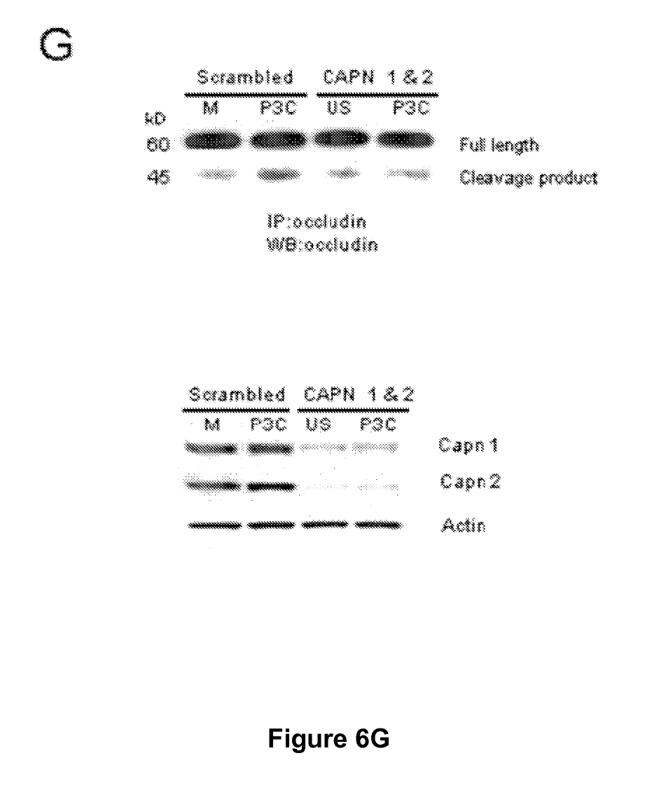

To determine if the association of calpain and occludin results in occludin cleavage, lysates of both cell lines (1HAEo-) and human small airway epithelial cells in primary culture (SAEC) were screened for generation of a 45 kDa occludin cleavage product predicted from studies of occludin processing in other model systems (Wu et al, 2000; Wan et al, 2000). While this cleavage product was not detected in unstimulated airway cells, it was readily identified by immunoblot by 1 hour post stimulation with P. aeruginosa or P3C (FIG. 6A and FIG. 6B).

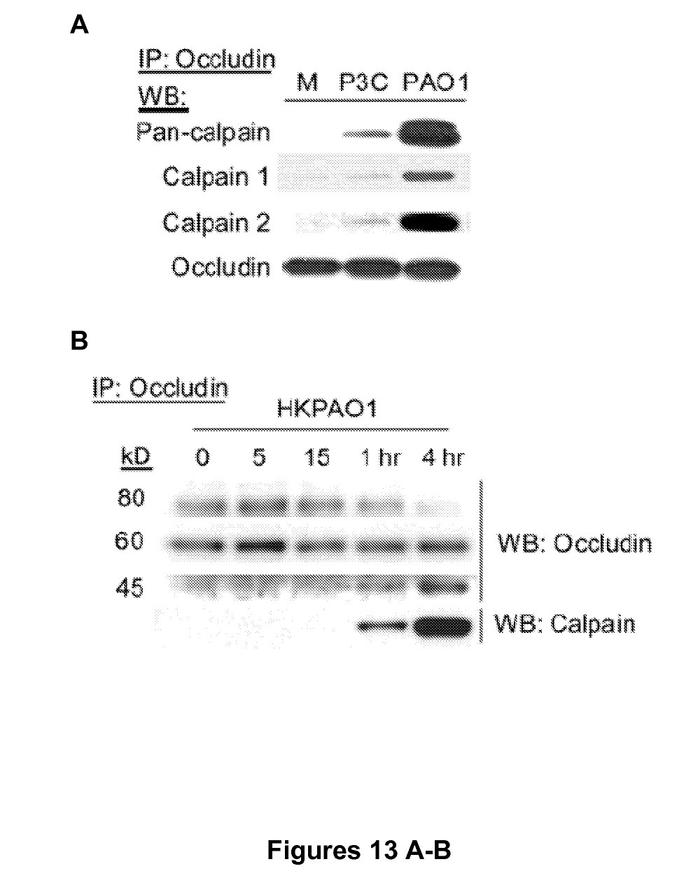

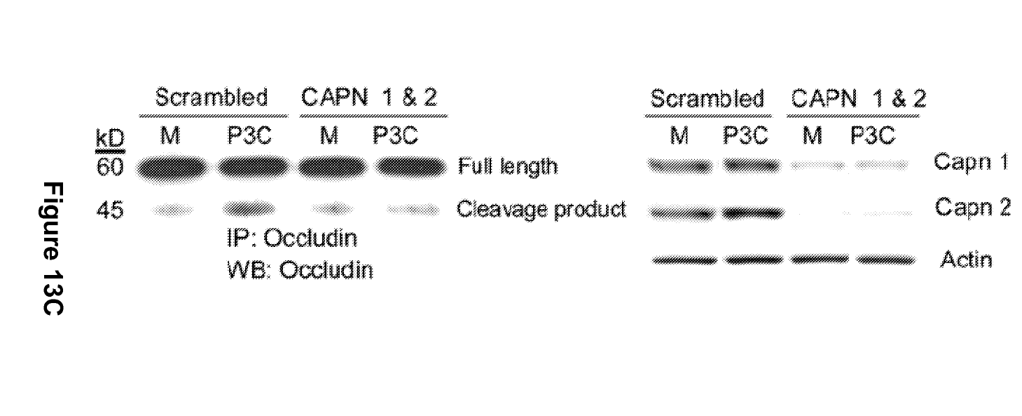

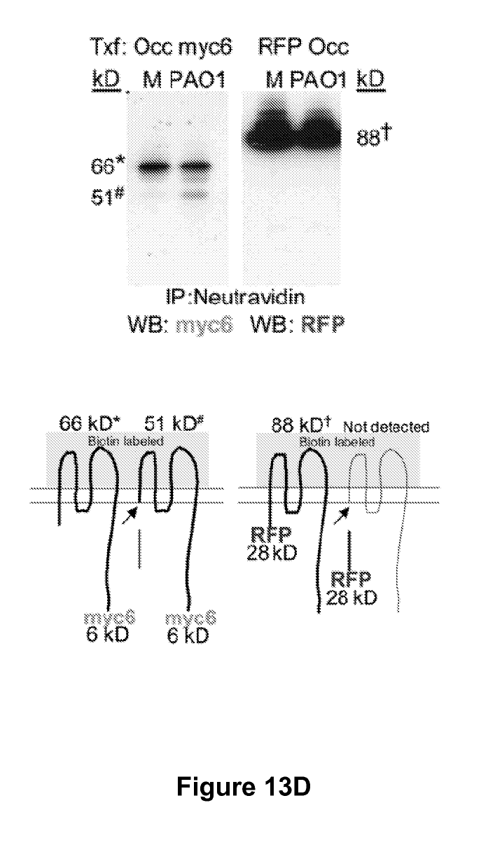

Immunoprecipitation experiments further confirmed this interaction and also demonstrated that both calpain 1 and calpain 2 associate with occludin (FIG. 13A). The association of occludin and calpain after 1 and 4 hr stimulation with P. aeruginosa PAO1 corresponded to a loss of the 80 kDa hyperphosphorylated form of occludin and subsequent appearance of a 45 kDa occludin cleavage fragment, predicted from studies of occludin processing in other model systems (FIG. 13B) (Wan et al., 2000; Wu et al., 2000). This result was also confirmed in human SAECs. Generation of the cleavage product was substantially decreased in cells unable to activate TLR2 signaling, in cells expressing a dominant negative TLR2 mutation lacking the tyrosine residues necessary for TLR2 phosphorylation (FIG. 6C), or in cells treated with an intracellular Ca²⁺ chelator BAPTA/AM (FIG. 6D) (Chun et al, 2006). In contrast to the calpain inhibitor ALLN, neither the general matrix metalloproteinase inhibitor GM6001 nor the caspase inhibitor Z-DEVD-FMK blocked generation of the occludin cleavage product (FIG. 6E). To further confirm the role of calpain in occludin cleavage, calpain 1 and calpain 2 expression were silenced by siRNA. The occludin cleavage product was detected in cells expressing scrambled oligos in response to P3C stimulation but not in cells expressing both calpain 1 and 2 siRNA (FIG. 6F, FIG. 6G and FIG. 13C). Knockdown of calpain 1 or calpain 2 individually was not sufficient to block occludin cleavage in response to P3C stimulation. The siRNAs (siGENOME ON-TARGETplus SMARTpool duplex) used against Calpain 1 were: Calpain 1 siRNA Sense Sequence 1 [GGAACAACGUGGACCCAUAUU (SEQ ID NO: 27)]; Calpain 1 siRNA Antisense Sequence 1 [5′-PUAUGGGUCCACGUUGUUCCUU (SEQ ID NO: 28)]; Calpain 1 siRNA Sense Sequence 2 [GUGAAGGAGUUGCGGACAAUU (SEQ ID NO: 29)]; Calpain 1 siRNA Antisense Sequence 2 [5′-PUUGUCCGCAACUCCUUCACUU (SEQ ID NO: 30)]; Calpain 1 siRNA Sense Sequence 3 [UAGAGACCAUGUUCCGAUUUU (SEQ ID NO: 31)]; Calpain 1 siRNA Antisense Sequence 3 [5′-PAAUCGGAACAUGGUCUCUAUU (SEQ ID NO: 32)]; Calpain 1 siRNA Sense Sequence 4 [GAAGGGCCAUGCCUACUCUUU (SEQ ID NO: 33)]; Calpain 1 siRNA Antisense Sequence 4 [5′-PAGAGUAGGCAUGGCCCUUCUU (SEQ ID NO: 34)]. The siRNAs (siGENOME ON-TARGETplus SMARTpool duplex) used against Calpain 2 were: Calpain 2 siRNA Sense Sequence 1 [CUACCAAGCUGUUCCGGAAUGAUUU (SEQ ID NO: 35)]; Calpain 2 siRNA Antisense Sequence 1 [5′PAUCAUCGACAGCUUGGUAGUU (SEQ ID NO: 36)]; Calpain 2 siRNA Sense Sequence 2 [CCGAGGAGGUUGAAAGUAAUU (SEQ ID NO: 37)]; Calpain 2 siRNA Antisense Sequence 2 [S′PUUACUUUCAACCUCCUCGGUU (SEQ ID NO: 38)]; Calpain 2 siRNA Sense Sequence 3 [GGAACUACCCGAAACCAACCAAUUUUUU (SEQ ID NO: 39)]; Calpain 2 siRNA Antisense Sequence 3 [5′PAAUGUGUUCGGGUAGUUCCUU (SEQ ID NO: 40)]; Calpain 2 siRNA Sense Sequence 4 [UGGAAACGCUAAUUUCCAAAGGAAUUUU (SEQ ID NO: 41)]; Calpain 2 siRNA Antisense Sequence 4 [5′PAUCUUGAAUAGCGUUUUUCCCCAUU (SEQ ID NO: 42)]

To locate where calpain cleaves occludin, a series of mapping experiments were done. Because the 45 kDa cleavage fragment of occludin was immunoprecipitated and immunoblotted using an antibody that recognizes the intracellular C-terminal tail of occludin, this data indicated that calpain cleaves at the intracellular N-terminal tail of occludin. To further map where calpain cleaves occludin, occludin constructs with N-terminal RFP (RFP Occ) (Shen and Turner, 2005) and C-terminal myc6 (Occ myc6) labels (Bojarski et al., 2004) were used. By biotinylating 1HAEo-cells expressing these labeled occludins and performing immunoblots on neutravidin precipitates with anti-RFP and anti-myc antibodies, the region of occludin targeted by calpain at the cell surface was identified. Occ myc6 is a 66 kDa protein that generates a 51 kDa myc-tagged fragment in heat-killed PAO1-treated cells (FIG. 13D). The generation of this tagged fragment indicates that the cleavage site is in the region of the intracellular N-terminal domain, which is illustrated in FIG. 13D. Expression of the N-terminal RFP-tagged occludin in airway cells results in a 88 kDa product that does not generate an RFP-tagged fragment, indicating that the RFP label has been cleaved and is not detectable at the cell surface (FIG. 13D). These studies show that calpain targets the N-terminal portion of occludin.

### Example 5

### Calpain Mediated Cleavage of E-Cadherin

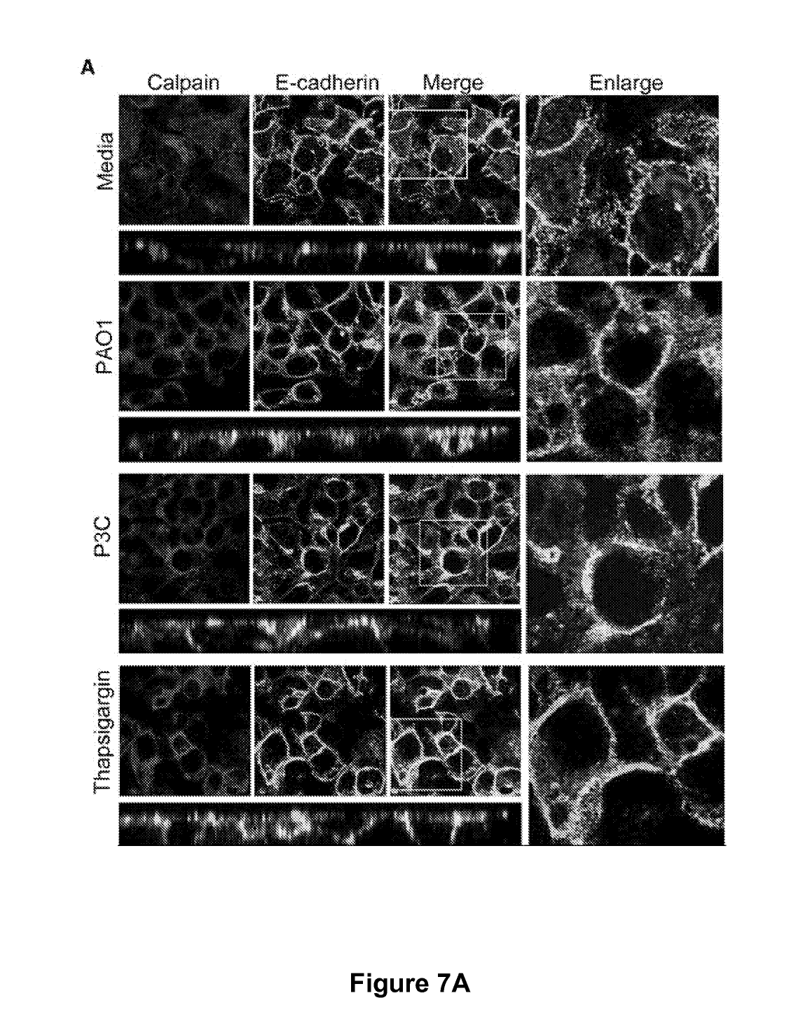

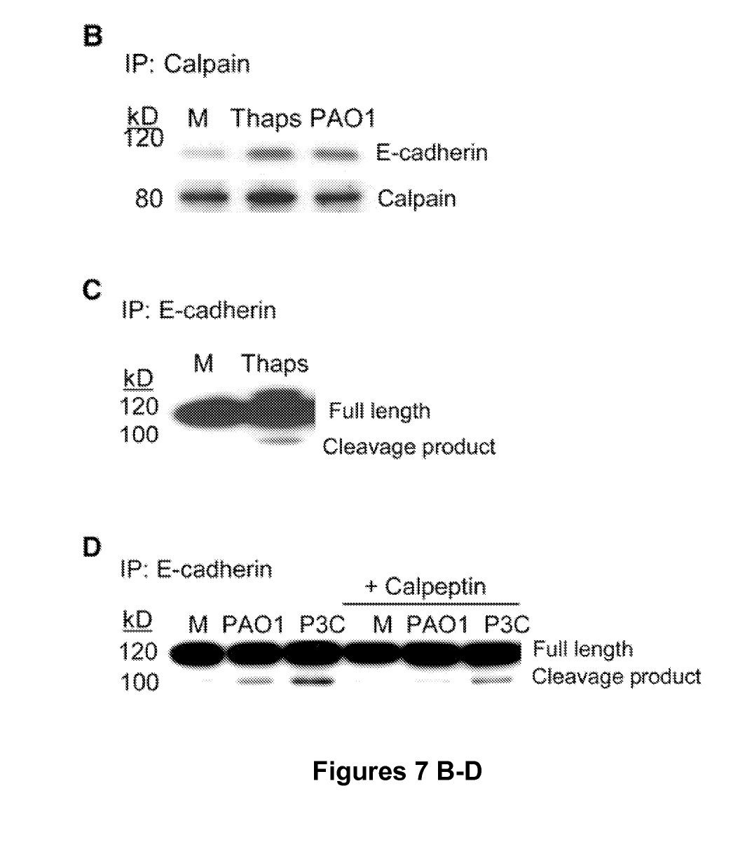

Confocal imaging and co-immunoprecipitation experiments were performed to determine if calpain targets E-cadherin. E-cadherin was predominantly membrane-associated in unstimulated airway cells (FIG. 7A). Calpain was mobilized to the membrane, co-localizing with E-cadherin following bacterial or P3C stimulation (FIG. 7A). Treatment of the cells with thapsigargin to generate the release of Ca²⁺ from intracellular stores also induced mobilization and co-localization of calpain with E-cadherin (FIG. 7A). The association of calpain and E-cadherin was further confirmed by immunoprecipitation (FIG. 7B) and calpain activity demonstrated by the generation of a 100 kDa E-cadherin cleavage product in thapsigargin, bacterial or P3C treated airway cells. The generation of the 100 kDa was substantially decreased in cells treated with the calpain inhibitor calpeptin (FIG. 7C and FIG. 7D). Thus, calpain activity induced by bacteria, or by intracellular Ca²⁺ fluxes results in E-cadherin cleavage.

### Example 6

### Calpain Activation Facilitates PMN Transmigration and Pulmonary Inflammation

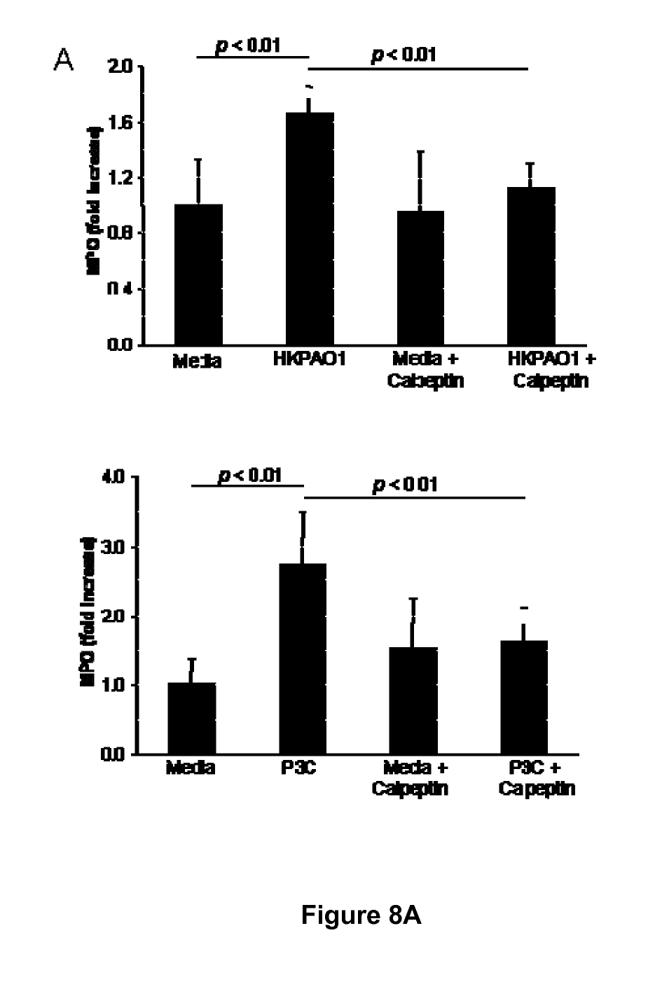

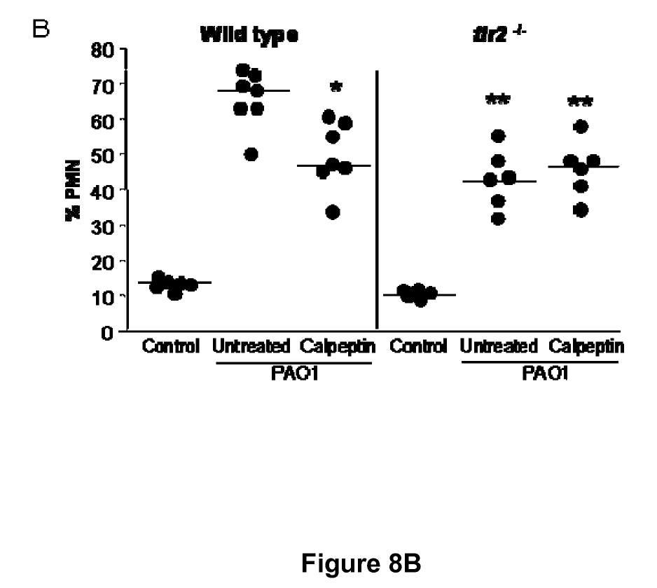

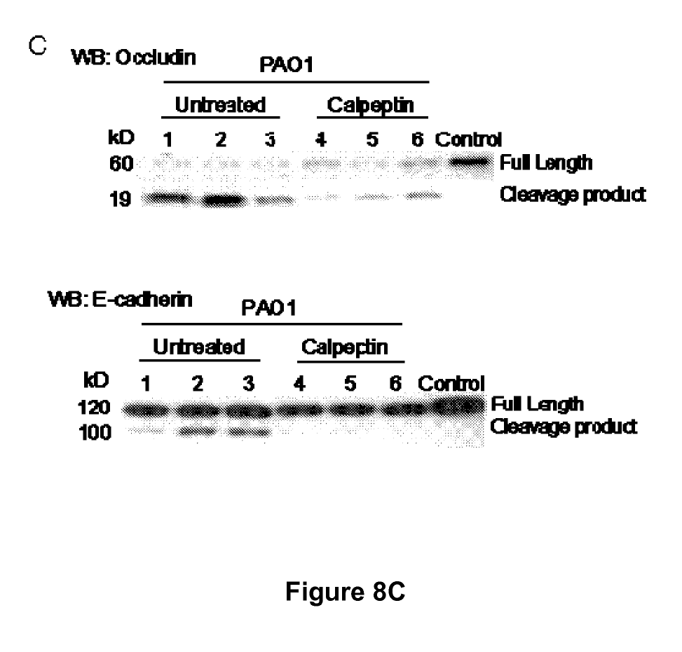

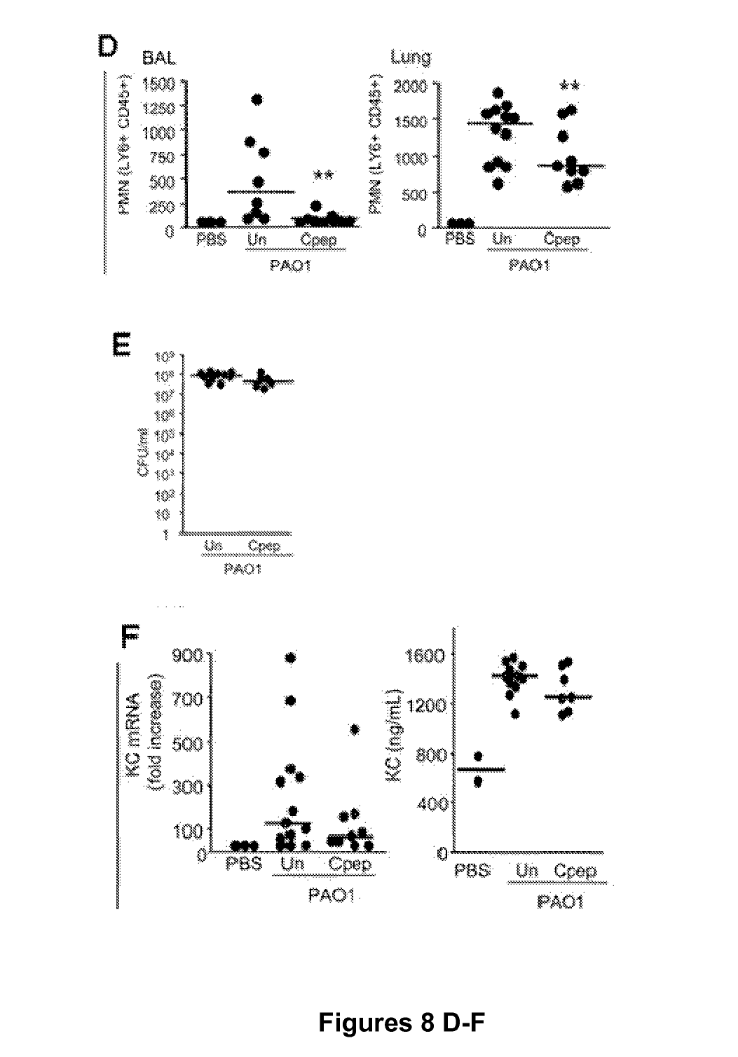

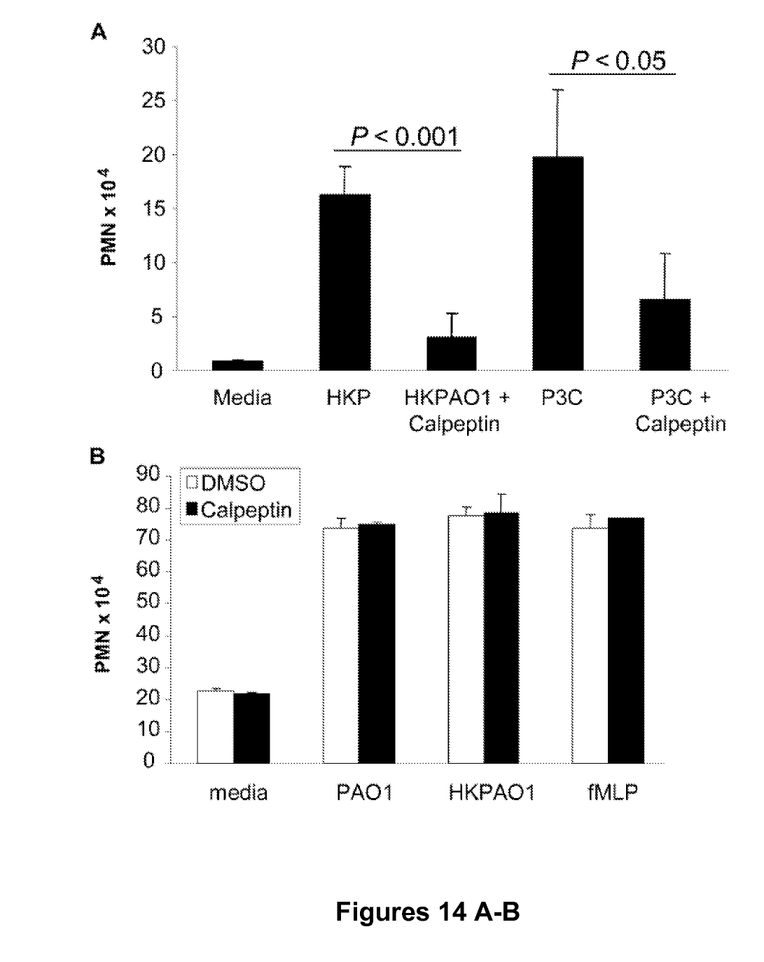

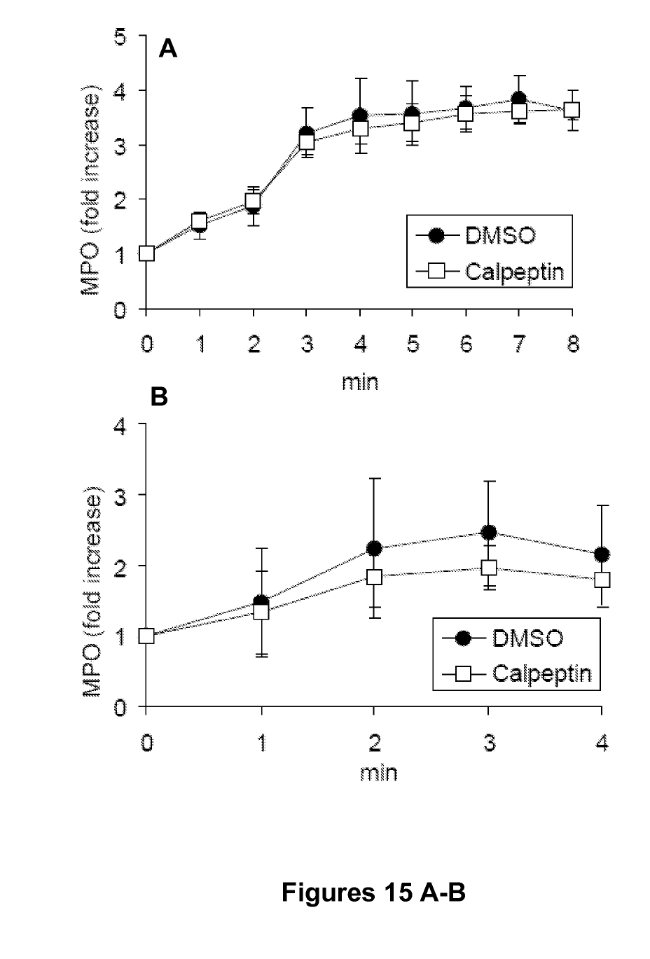

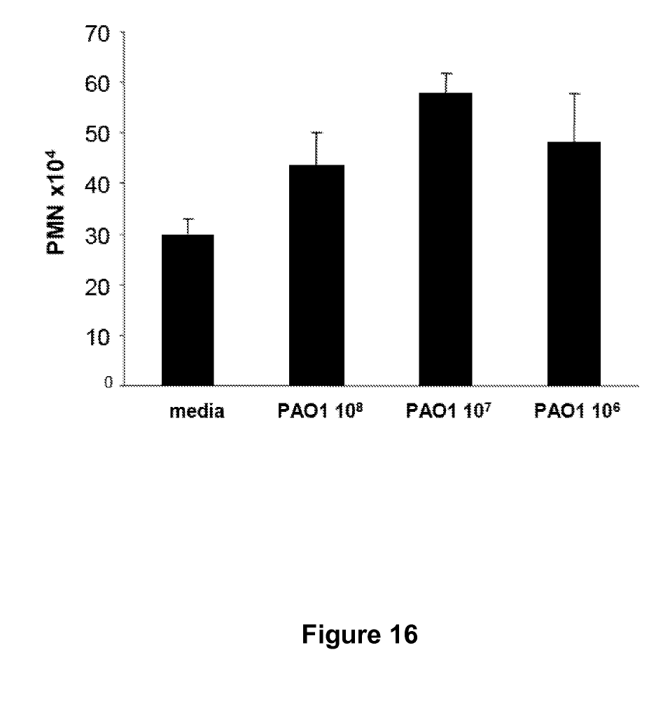

As a major function of TLR2 signaling is stimulating chemokine expression and PMN recruitment, calpain may modify junctional proteins to facilitate migration of PMNs across the epithelial barrier. To demonstrate the role of calpain in PMN transmigration from the basal to the apical surface of polarized human airway epithelial cells, monolayers were stimulated with heat killed P. aeruginosa PAO1 or P3C in the presence of the calpain inhibitor calpeptin. PMN migration was monitored by measuring myeloperoxidase activity in the apical compartment. Both heat killed P. aeruginosa PAO1 induced (p<0.001) and P3C-induced (p<0.05) PMN transmigrations were inhibited in airway cells treated with calpeptin (FIG. 8A and FIG. 14A). Calpeptin did not directly inhibit migration of PMNs across a porous Transwell (without epithelial cells) in response to live P. aeruginosa PAO1, heat-killed PAO1, or the chemoattractant fMLP (FIG. 14B), nor did calpeptin inhibit the activity of myeloperoxidase or the production of myeloperoxidase by PMNs (FIGS. 17A and 17B).

The biological importance of TLR2 induced calpain activity was tested in both neonatal and adult murine models of infection. In 7-day old murine models of intraperitoneal (i.p.) calpeptin, treatment resulted in 31% fewer PMNs recruited into the lung (p<0.05; FIG. 8B) 4 hours following intranasal P. aeruginosa inoculation as compared to vehicle-treated mice (FIG. 8B). Significantly fewer PMNs (p<0.01; FIG. 8B) were mobilized into the lungs in tlr^{-/-} mice in response to infection as compared with wild-type (WT) infected mice, a response equivalent to that of the calpeptin-treated mice. PMN recruitment in the tlr2^{-/-} animals was not further diminished by calpain inhibition. The involvement of calpain-dependent cleavage of junctional proteins was verified by identification of the presence of occludin and E-cadherin cleavage products in whole lung lysates of the infected wild type mice. The cleavage products were not observed in uninfected controls and they were substantially decreased in the calpeptin treated animals (FIG. 8C). Amounts of the murine chemokine KC in lung lysates which were comparable in tlr2^{-/-} and wild type mice, indicating that both the tlr2^{-/-} mice and wild type calpeptin treated mice had sufficient chemokine production to recruit PMNs.

To test the effects of calpain inhibition on the transmigration of PMNs into P. aeruginosa-infected airways, bronchoalveolar lavage (BAL) experiments were performed on adult C57BL/6 mice. Mice were treated with i.p. calpeptin or vehicle and intranasally infected with P. aeruginosa for 2 hr. PMNs from BAL or whole-lung suspension were quantified by flow cytometry. Compared to vehicle-treated controls, calpeptin-treated mice had 37% fewer PMNs recruited into the whole lung and 90% fewer into the airway lumen, indicating a more pronounced effect on the epithelial junctions (FIG. 8D). Calpeptin treatment did not affect the bacterial load or CXCL1 (KC) levels in the lung at this time point (FIGS. 8E and 8F). Thus, recruitment of PMNs into the murine lung and airway lumen in response to P. aeruginosa is facilitated by calpain activity and its effects on junctional proteins at the mucosal surface.

Discussion

Mucosal epithelial cells initiate the host response to inhaled pathogens through chemokine and cytokine production which recruit PMNs into the airway lumen to eradicate the infecting bacteria. The TLRs are important in the host defenses against pulmonary infection and P. aeruginosa infection specifically (Skerrett et al, 2004). The data presented herein indicate that TLR2 signaling also initiates the efferent limb of the inflammatory pathway by affecting changes in epithelial junctions to accommodate PMN egress into the airways. TLR2 is especially important in the airway; it is apically displayed on airway cells and broadly responsive to diverse bacterial ligands including lipoproteins, cell wall components and pili, through its association with lipid co-receptors (Adamo et al, 2004; Soong et al, 2004). While TLR2 is not the only TLR involved in sensing luminal pathogens, it appears to be especially important in the initial stages of the innate immune response.

An immediate consequence of TLR2 activation is the local generation of Ca²⁺ fluxes which are both sufficient and necessary to evoke the distal proinflammatory signaling cascade, NF-κB activation, and IL-8 production (Chun et al, 2006). Ca²⁺ fluxes also activate calpains which then target junctional proteins to facilitate PMN recruitment. Thus, TLR2-mediated Ca2+ release also increases PMN transepithelial migration into the airway lumen.

The major PMN chemokine, IL-8, was shown to recruit PMNs from the bloodstream to the basolateral surface of airway epithelial cells but was not involved in further mediating PMN transmigration into the airway lumen (Hurley et al., 2004). In this regard, the secretion of an arachidonic acid metabolite, hepoxilin A3, by airway epithelial cells has been proposed to direct PMN migration across airway epithelial cells (Hurley et al., 2004). Although guided by chemokines and chemoattractants, PMNs still need to migrate across a complex network of tight and adherens junction proteins. These results demonstrate a dramatic loss in occludin and E-cadherin localization at the membrane in response to P3C and PAO1. There are no changes in transepithelial resistance, dextran permeability, or bacterial invasion, showing that these modifications to the junctions are subtle but sufficient to facilitate PMN transmigration. A qualitative change in the junction in response to P3C, PAO1, and thapsigargin is demonstrated by the increase in accessibility of the junctions to biotin, a 557 Da molecule. Much of what is known about PMN transepithelial migration comes from studies using intestinal epithelial cells, which show that focal disruption of epithelial TJs facilitates PMN egress (Burns et al., 2003). The results shown herein demonstrate that TLR2-dependent Ca2+ fluxes signal the activation of calpains, which subsequently target junction proteins to increase PMN transepithelial migration.

Calpains are ubiquitously expressed Ca²⁺-dependent proteases, and can be activated by ATP or PKC induced Ca²⁻ fluxes in the micromolar (mu-calpain or calpain 1) or millimolar (m-calpain or calpain 2) range (Goll et al, 2003). When membrane associated, this Ca²⁺ requirement is diminished to a more physiological range (Shao et al, 2006) as occurs when calpains are mobilized to the epithelial junctions. Calpains are important in a number of physiological processes through their effects on integrins, platelet activity and especially cell adhesion and migration (Franco et al, 2005; Glading et al, 2002; Kuchay et al, 2007). Calpains target cytoskeletal proteins in migrating cells such as talin, ezrin and focal adhesion kinase (Franco et al, 2005) and participate in cellular detachment and polarity during migration (Fanco et al, 2005; Gladin et al, 2002; Nuzzi et al, 2007). EGFR and ERK signaling can activate calpains (Shao et al, 2006). The involvement of calpain 2 in regulating the cytoskeletal architecture of the lung was reported over a decade ago in cells exposed to phorbol esters (Dweyer-Nield et al, 1996). A similar role for calpains in response to more physiological stimuli and the generation of Ca²⁺ fluxes associated with TLR2 activation was observed. Other known cytoskeletal targets of calpain, such as ezrin or talin, can also be targeted in airway epithelial cells as a consequence of TLR2 activity (Franco and Huttenlocher, 2005).

The importance of proteases, especially the matrix metalloproteinases, in inflammatory processes is well established (Parks et al, 2004). MMP7 targets several epithelial components (Li et al, 2002) including E-cadherin and contributes to the shedding of its ectodomain and endocytosis (McGuire et al, 2003). MMP9 facilitates translocation of PMNs from endovascular spaces causing MMP9^{-/-} mice to have defective PMN trafficking in response to infection (Ichiyasu et al, 2004). Occludin proteolysis is also reported to be a component of PMN-dependent inflammation (Wachtel et al, 1999). However, the observation that occludin proteolysis remains unaltered in the MMP9^{-/-} mouse (Ichiyasu et al, 2004) indicated that additional protease(s) must target this tight junction protein. The failure of general protease inhibitors to block occludin or E-cadherin cleavage in airway cells, as demonstrated herein, also indicates the involvement of a different protease. While bacterial proteases or toxins can affect epithelial junctions, the ability of heat killed organisms or the synthetic ligand P3C to induce modifications of the junctions indicates that epithelial not bacterial proteases are sufficient.

The results shown herein indicate that epithelial calpain has a major role in targeting junctional proteins and facilitating PMN transmigration. In both our in vitro and in vivo models of infection, inhibition of epithelial calpain activity blocked the ability of PMNs to traverse the epithelium. To document that this result was not due to inhibition of PMN movement, the results described herein demonstrate that there is no effect of calpeptin on PMN chemotaxis across porous Transwells. It has been shown that constitutive calpain activity in resting PMNs blocks their ability to migrate, and calpain inhibition was shown to increase PMN movement by initiating MAPK and Rac GTPase activation (Katsube et al., 2008; Lokuta et al., 2003). Thus, epithelial calpain activity is independent from that of the PMN itself.

The contribution of calpain activity in the epithelium seems especially important in the egress of PMNs from the lung into the airway lumen. While TLR2-mediated calpain activation appears to account for only 30%-40% of PMN recruitment into the lung, calpain inhibition blocked the majority (90%) of PMN mobilization into the airway lumen, demonstrating the importance of calpain activity in PMN egress across the airway epithelium. The remaining TLR2-independent signaling can be due to the TLR5 ligand, flagella, which also activates Ca2+ fluxes in airway cells (Ratner et al., 2001).

Occludin was one of the first components of tight junctions to be identified but its precise role in maintaining or regulating the epithelial barrier remains incompletely defined (Feldman et al, 2005). Occludin interacts with many components of the junctional complex (Feldman et al, 2005) and binds directly to the ZO-1 scaffold, diffuses rapidly within the TJs at steady state (Shen et al., 2008), and internalizes in response to various stimuli (Yu and Turner, 2008). A decrease in the phosphorylated form of occludin was observed. This phosphorylated from is the predominant form of occludin expressed at the TJs (Feldman et al., 2005). The kinetics of this decrease coincided with the association of occludin with calpain and the appearance of a 45 kDa fragment of occludin. The results described herein show that the phosphorylated 80 kDa form of occludin is the main target for calpain, as the levels of the nonphosphorylated 60 kDa form of occludin are unchanged in response to stimulation.

The occludin null mouse has pleiotropic alterations in inflammatory responses (Saitou et al, 2000). Paracellular permeability to specific compounds is affected but transepithelial resistance (TER) is maintained and there is no real defect in the tight junction barrier (Yu et al, 2005). The results described herein demonstrate displacement of occludin from junctions in response to TLR2 signals without defects in TER or permeability to dextrans. These observations show that calpain mediated processing of occludin can interrupt interactions with cytoskeletal binding partners and influence the ability of the cytoskeleton to accommodate PMN transmigration without breaching the integrity of the epithelial barrier. The results described herein also show that calpain targets the N-terminal intracellular domain of occludin.

E-cadherin is a known calpain substrate with calpain cleavage sites on its cytoplasmic tail between residues 782 and 787 (Rios-Doria et al, 2003). The 100 kDa truncated forms of E-cadherin generated by epithelial calpain in airway cells have been reported previously in other model systems (Rios-Doria et al, 2005). E-cadherin processing can be accomplished by a number of proteases (D'Souza-Schorey et al, 2005) and is an important component of constitutive E-cadherin endocytosis and recycling (Bryant et al, 2004; Bryant et al, 2007). There are numerous E-cadherin binding partners in the junctional complex and these interactions can have effects on endothelial permeability (Mehta et al, 2006). E-cadherin associates directly with β-catenins and hence to actin and the cytoskeleton (Mehta et al, 2006). Mutant VE-cadherin lacking the extracellular domain results in impaired PMN migration in response to chemotactic stimuli (Orrington-Meyers et al, 2006). Interactions of cadherins and the small GTPases that regulate actin polymerization affect paracellular permeability in endothelial cells (Hordijk et al, 2003). Calpain mediated cleavage of epithelial E-cadherin, along with occludin, can ultimately affect the deformability of the cytoskeleton to enable the paracellular junction to accommodate migrating PMNs.

While TLR2 is not considered the major innate immune effector for Gram negative organisms, at least for P. aeruginosa, TLR2 activation in the airway epithelium coordinates both the afferent and efferent limbs of the initial inflammatory response. Not only does the airway epithelial cell produce IL-8 to direct PMN recruitment, the same signaling cascade modulates the TJ to accommodate PMN egress without breaching the epithelial barrier. This pathway provides a useful pharmacologic target in pulmonary infection to selectively limit PMN recruitment into the lung, without entirely compromising host defenses to bacterial infection.

Methods—Cell Lines and Bacteria

Human airway epithelial cell lines, 1HAEo- and 16HBE cells (D. Gruenert, California Pacific Medical Center Research Institute, San Francisco, Calif.), were grown as previously detailed (Ratner et al, 2001; Rajan et al, 2000). Human small airway epithelial cells in primary culture (SAEC) were obtained from (Lonza/Clonetics) and grown as directed. The TLR2 WT, TLR2 Y616A/Y761A (TLR2 YY) mutant expressing cells and TLR2 siRNA cell lines were generated as previously described (Chun et al, 2006). P. aeruginosa PAO1 resuspended in minimum essential media (MEM) at a density of 10⁸ CFU/ml was heat killed by incubating at 60° C. for 1 h.

Methods—Confocal Microscopy

16HBE cells were grown on 3 μm pore size Transwell-Clear filters (Corning-Costar) with an air-liquid interface to form polarized monolayers. For confocal imaging, cells were fixed with 4% paraformaldehyde, blocked with 5% normal donkey serum and incubated at 4° C. overnight with the polyclonal pan-calpain (Santa Cruz Biotechnology), monoclonal occludin (Invitrogen/Zymed) and/or monoclonal E-cadherin (BD Pharmingen) antibodies. Alexa Fluor 594 conjugated and Alexa Fluor 488 conjugated secondary antibodies (Invitrogen/Molecular Probes) were added at room temperature for 1 h. After washing, filters were removed from Transwells using a scalpel, mounted with Vectashield with DAPI (Vector Laboratories) onto glass slides and imaged using a Zeiss LSM 510 Meta scanning confocal microscope. For biotin labeling experiments 1 mg/ml EZ-Link Sulfo-NHS-LC-Biotin was added apically for 30 min at 4° C. before fixing the cells. Alexa Fluor 555 conjugated streptavidin (Molecular Probes) was added for detection of biotin.

Methods—Transepithelial Resistance and Dextran Permeability Assays

Polarized 16HBE cells grown in 12 mm Transwell-Clear filters (Corning-Costar) were stimulated apically with 10⁷ CFU of heat killed PAO1. Transepithelial resistance readings were taken using a Millipore resistance reader which applies a 20 μA square wave alternating current across the monolayers at 12.5 Hz. The resistance of Transwell inserts without cells was used as a baseline control. After 5 h of stimulation, Alexa Fluor 488 labeled dextran (10,000 MW) was added apically for 1 h and fluorescence in the basal compartment monitored at ex 485, em 535 with a SpectraFluor Plus fluorimeter (Tecan). As a positive control, cells were treated apically and basolaterally for 1 h with 0.02% EGTA, an extracellular Ca²⁺ chelator which increases paracellular permeability by disrupting cadherin mediated cell-cell adhesion.

Methods—Bacterial Transmigration Assay

Polarized 16HBE cells were pretreated with media alone, heat killed PAO1 (10⁷ CFU), P3C (15 μg/ml), or thapsigargin (0.1 μM) for 4 h. Live PAO1 (2×10⁷ CFU) were added apically for 1 h and media from the basal compartment collected and plated on LB agar plates. Bacterial transmigration across monolayers pretreated with 0.02% EGTA was used as a positive control.

Methods—Calpain Assay

Confluent monolayers of 1HAEo-cells or SAECs were loaded with 20 μM t-BOC-L-leucine-L-methionine amide (Boc-LM-CMAC), a calpain specific membrane permeable fluorogenic substrate. Cells were stimulated with heat killed PAO1 (10⁷ CFU) or P3C (15 μg/ml) and fluorescence was quantified at ex 360 nm and em 465 nm with a Spectrafluor Plus fluorimeter (Tecan).

Methods—Membrane Preparations

Membrane preparations were performed as previously described (Lopez-Santiago et al, 2006). 1HAEo-cells were rinsed and lysed in Tris-EGTA buffer (50 mM Tris-Cl, pH 8.0 with NaOH, and 10 mM EGTA) containing Complete Mini protease inhibitor tablets (Roche) at twice the recommended concentration. The cells were homogenized and centrifuged at 3000×g for 5 min at 4° C. to remove nuclei. The supernatant was ultra-centrifuged at 4° C. for 10 min at 195,000×g, and the pellets resuspended in Tris-EGTA buffer containing Complete Mini protease inhibitor tablets.

Methods—Immunoprecipitation and Immunoblotting

1HAEo-cells or SAECs were grown to confluence on 10 cm plates, stimulated with heat killed PAO1 (10⁷ CFU) or P3C (15 μg/ml), and whole cell lysates made using 60 mM n-octyl-β-D-glucopyranoside (OGP) in TBS (0.1 M Tris-HCl and 0.15 M NaCl, pH 7.8) containing Complete Mini protease inhibitor tablets (Roche), 1 mM sodium orthovanadate and 100 mM sodium fluoride. Monoclonal anti-E-cadherin (BD Pharmingen), polyclonal anti-pan calpain (Santa Cruz Biotechnologies) or monoclonal anti-occludin (Invitrogen/Zymed) antibodies were used for immunoprecipitations followed by the addition of Protein G Agarose beads (Invitrogen) (Chun and Prince, 2006). Immunodetection was performed using monoclonal anti-E-cadherin, polyclonal anti-occludin, anti-pan calpain, anti-calpain 1 and anti-calpain 2 antibodies (Santa Cruz Biotechnology).

Methods—Calpain siRNA

1HAEo-cells were transiently transfected with scrambled (control) or calpain 1 and 2 siRNA Smart pool oligos (Dharmacon) (FIGS. 11 and 12). Transfection was carried out using an Amaxa Nucleofector following manufacturer's instructions. Briefly, 10⁶ 1HAEo-cells were electroporated in 100 μl Buffer L with 1.5 μg of scrambled or siRNA oligo and transferred into pre-warmed 6 well tissue culture dishes. Cells were incubated at 37° C., 5% CO₂ for 48 hours before performing experiments.

Methods—Neutrophil Isolation and Migration Assays

Human PMNs were isolated according to standard techniques from venous blood from healthy consenting adults in accordance with a protocol approved by the Institutional Review Board of Human Subjects at Columbia University (IRB-AAAC5450). PMNs were isolated using dextran sedimentation and Hypaque-Ficoll (Sigma) density-gradient separation, followed by hypotonic lysis of erythrocytes as previously described (Boyum et al, 1968). Purified PMNs were resuspended in HBSS with Ca²⁺ and Mg²⁺ before use in migration experiments. Migration assays were performed across 16HBE monolayers plated on the underside of 3 μm transwells which were coated with minimal essential media containing 0.01 mg/ml fibronectin, 0.03 mg/ml bovine collagen type I, 0.1 mg/ml BSA. After allowing the cells to adhere overnight, transwells were flipped over and inserted into 24-well plates. 500 μl of media was added to the lower chamber (apical surface of the cell) and 100 μl of media added to the transwell (basolateral surface of the cell). When the cells became confluent, they were polarized with an air liquid interface for 5 days by removing the 500 μl of media in the lower chamber. Selected wells were preincubated for 30 min with 20 μM calpeptin in the apical and basolateral chambers. Monolayers were stimulated with 250 μl heat killed PAO1 (10⁷ CFU) or P3C (15 μg/ml) at the apical surface (lower chamber) for 4 h at 37° C. Media in the basolateral chamber (in the transwell) containing calpeptin was removed and 10⁵ neutrophils (in 50 μl) were then added to the basolateral surface for 2 h. Simultaneously, calpeptin was added to the apical chamber to a final concentration of 20 μM to maintain epithelial exposure to inhibitor. Neutrophils from the apical chamber were collected and centrifuged for 6 min at 1500 rpm at 4° C. Supernatant was discarded and the cell pellet resuspended in 50 mM sodium phosphate pH 7.0. The cell suspension was freeze thawed three times and stored at −80° C. until used for myeloperoxidase assay.

Methods—Myeloperoxidase Assay

Myeloperoxidase (MPO) activity was measured as described in the R&D recombinant human MPO activity assay protocol. Briefly, 20 μl of the cell suspension was added to 30 μl of 0.00667% H₂O₂ and 50 μl of 100 mM guaiacol in a 96-well plate and t oxidized guaiacol was read on a microplate reader at 450 nm. A standard curve was made using serially diluted recombinant human MPO.

Methods—Mouse Model of Infection

Seven day old C57BL/6 wild type or tlr2^{-/-} mice (Jackson Laboratories) were intranasally inoculated with 10 μl PAO1 (10⁸ CFU) (Tang et al, 1996). Control mice received 10 μl of PBS. Mice were pretreated with 20 mg/kg calpeptin or DMSO in PBS by intraperitoneal (i.p.) route 16 h and 2 h before intranasal inoculation and a third dose was administered by i.p. route 30 min post infection. Lungs were harvested 4 h after infection and cell suspensions used for PMN detection and immunoblotting. For neutrophil detection, lung cell suspensions were double stained with phycoerythrin (PE)-labeled anti-CD45 (to detect leukocytes) and fluorescein isothiocyanate (FITC)-labeled anti-Ly6G antibodies (to detect neutrophils) (BD Pharmingen) and analyzed by flow cytometry with a FACSCalibur using Cell Quest software (Becton Dickinson) (Gomez et al, 2004). Irrelevant, isotype-matched antibodies were used as a control. Cells were gated on the basis of their forward scatter and side scatter profile and analyzed for double expression of CD45 and Ly6G. Immunoblots were performed on 10 μg of protein obtained by lysing lung cells in OGP buffer as described herein for 1HAEo-cells. Mice protocol number AAAA5999 was approved by the Institutional Animal Care and Use Committee at Columbia University.

Methods—Statistical Analysis

Samples with normal distribution were analyzed by Student's t test. Mouse samples that did not follow normal distribution were compared using the non-parametric Mann-Whitney test. Differences between groups were considered significant at P<0.05. Statistical analysis was determined using GraphPad Instat version 3.0 (GraphPad).

Methods—Real Time PCR of Calpains and Calpastatin

1HAEo-cells were grown in 6-well plates to confluence. After incubation with media alone, heat-killed PAO1 or P3C, cells were lysed and RNA was isolated using the Qiagen RNeasy Mini Kit. cDNA was made from 1 μg of RNA using an iScript synthesis kit (Bio-Rad). For quantitative real-time PCR of calpain 1, calpain 2 and calpastatin, cDNA amplification was performed in a Light Cycler using the DNA Master SYBR Green I kit (Roche) according to the manufacturer's instructions. Primers used for calpain 1 amplification were 5′-TGCGAGAGGTCAGCACCCGC-3′ (SEQ ID NO: 19) and 5′-CAGGTCAAACTTCCGGAAGATGG-3′ (SEQ ID NO: 20). The primers used for calpain 2 amplification were 5′-ATCTGCCAAGGAGCCCTAGG-3′ ((SEQ ID NO: 21) and 5′-TAGTGTTCCAGCTTGGGCAG-3′ ((SEQ ID NO: 22). The primers used for calpastatin were 5′-AAAGATGGAAAACCACTATTGCCAGAGC-3′ (SEQ ID NO: 23) and 5′-GACCTCTTCTAATCTATAATCAGGAGG-3′ (SEQ ID NO: 24). For calpain 1, calpain 2 and calpastatin quantification, 35 cycles were run with denaturation at 95° C. for 8 s, amplification at 50° C. for 15 s, and extension at 72° C. for 12 s. Amplification of human actin was used as a control for standardization. The primers used for human actin amplification were 5′-TCCTCCCTGGAGAAGAGCTAC-3′ (SEQ ID NO: 25) and 5′-TAAAGCCATGCCAATCTCATC-3′ (SEQ ID NO: 26), and 35 cycles were run with denaturation at 95° C. for 8 s, amplification at 63° C. for 10 s, and extension at 72° C. for 12 s.

PMN Migration Assay with Live PAO1

The migration of PMN across polarized 16HBE monolayers in response to live PAO1 was tested as previously described (Hurley et al 2004). Briefly, inverted 16HBE monolayers were apically treated with various concentrations of PAO1 or media alone. After 2 h, DMEM was added to the apical chamber and 10⁶ PMNs in DMEM added to the basolateral chamber and incubated at 37° for 2 h. PMNs that migrated into the apical chamber were quantified by myeloperoxidase assay.

### Example 7

### Modulation of Transmembrane Proteins E-Cadherin and Occludin or the Actin-Plasma Membrane Linker Ezrin

The goals of this project are (1) to examine the participation of airway epithelial cells in the immune response to inhaled bacteria, (2) to determine how this process is involved in keeping the normal lung free of infection and (3) to identify what goes wrong in diseases such as cystic fibrosis. Ca²⁺ is an important second messenger in airway epithelial cells and an inherent component of epithelial proinflammatory signaling in response to bacterial ligands. The afferent portion of the epithelial signaling system functions to link bacterial stimuli to corresponding receptors leading to the subsequent activation of MAP kinases and NF-κB to induce epithelial chemokine and cytokine expression. This prophetic example will investigate an efferent pathway activated by Ca²⁺ fluxes that facilitates the movement of PMNs into the airway lumen to eradicate infection.

The signaling process that facilitates PMN migration through the epithelial barrier and how this process can be exploited by bacterial pathogens will be explored in the following specific aims: (1) The consequences of TLR2 signaling and associated calpain activity on epithelial barrier properties will be established by activating endogenous signaling pathways by conserved bacterial ligands to permit PMN migration through paracellular junctions without breaching the epithelial barrier. This can be examined by studying the mobilization of calpain to the epithelial junctions, defining the interactions of calpain with its targets occludin and ezrin and characterizing the trafficking of calpain substrates in response to bacterial stimuli. (2) The physiological consequences of calpain-modified junctional proteins on the barrier function of the epithelium can be defined by quantifying alterations in transepithelial resistance as well as effects on epithelial permeability to solutes, to bacteria and to PMN transmigration, both in vitro and in vivo. These results can demonstrate how epithelial junctions, the “Achilles heel” of the mucosal immune system, are modulated by both the relatively avirulent organisms that cause chronic infection in CF as well as by the toxin secretors that target similar epithelial junctional components, to cause invasive infection.

TLR2 signaling generates Ca²⁺ second messengers. During the epithelial IL-8 cascade, 100 nM Ca²⁺ fluxes are rapidly initiated following the application of bacteria to monolayers of airway cells. Both Ca²⁺ fluxes and the application of bacterial to epithelial monolayers are necessary and sufficient to activate IL-8 production. Bacteria induce the release of 100 nM Ca²⁺ fluxes from intracellular stores initiating signaling that can also be activated by treating airway cells with thapsigargin, a SERCA pump inhibitor. Ca²⁺ fluxes were of similar amplitude and frequency in human airway cell lines as well as cells in primary culture. Because TLR2 was responsible for signal transduction across the infected epithelial surface and because activation of TLR2 by bacteria, antibody to the asialoGM1 co-receptor or the TLR2 agonist Pam₃Cys-Ser-Lys₄ (P3C) resulted in immediate Ca²⁺ fluxes, Ca²⁺ can be generated as a consequence of TLR2 activation.

Ligation of TLR2 is immediately followed by c-Src mediated phosphorylation of TLR2. This event stimulates recruitment of PI3K and PLCγ to the cell membrane, releasing Ca²⁺. Monitoring of NF-κB activation or measuring production IL-8 showed that cells treated with BAPTA/AM to chelate intracellular Ca²⁺ were unable to activate distal proinflammatory signaling. Similar results were observed in cells expressing siRNA to TLR2 expression and in cells expressing of a dominant negative mutant of TLR2 wherein the two sites of tyrosine phosphorylation are mutated.

Unlike TLR2 agonists, LPS does not activate Ca²⁺ fluxes or stimulate the mobilization of c-Src (or other signaling components) into the lipid raft. Thus, Ca²⁺ functions as an intracellular messenger for relaying proinflammatory responses to bacteria in airway cells and is linked to the activation of TLR2 on the exposed surface of airway cells.

Ca²⁺ fluxes are communicated from cell to cell via gap junctions. In vivo imaging techniques show that mechanical stimuli via gap junctions communicate Ca²⁺ fluxes from cell to cell in alveolar epithelial cells. The Ca²⁺ fluxes generated by TLR2 may similarly move via gap junctions to activate adjacent epithelial cells that are not directly stimulated by bacterial ligands. This can provide a mechanism to amplify epithelial signaling in response to a perceived infection. The gap junction protein connexin43 (Cx43) transmits Ca²⁺ fluxes from cell to cell. TLR2 positive epithelial cells transmit Ca²⁺ fluxes to TLR2 negative epithelial cells in response to a TLR2 agonist. This signaling is regulated by c-Src mediated phosphorylation of Cx43 which gates the Cx43 channel and limits the extent of Ca²⁺ signaling. Thus, Ca²⁺ not only functions to activate downstream responses in a single epithelial cell, it also facilitates cell-cell communication.

To determine if the transmembrane proteins E-cadherin and occludin or the actin-plasma membrane linker ezrin are modulated to facilitate PMN mobilization through tight junctions of airway cells, the effects of P. aeruginosa and the TLR2 agonist P3C were examined on polarized human airway cells grown at an air liquid interface in a polarized fashion. Two central issues were addressed: (1) Are specific epithelial junctional components modulated by the activation of TLR2 by bacteria? (2) Are these epithelial components common substrates for a Ca²⁺ activated protease, initiated by TLR2 activation.

TLR2 associated activation of calpain results in the modification of epithelial junctions and Calpain is recruited to TLR2/caveolin-1 associated lipid rafts

In the experiments described herein demonstrate that calpain is recruited into TLR2/caveolin-1 associated lipid rafts where it targets membrane associated occludin and ezrin. The biological significance of this cascade in PMN mobilization and details of this interaction (e.g., regulation of calpain activity, calpain targets, their sites of cleavage, the nature and effects of phosphorylation and endocytosis) are determined. In the second aim, modification of the distribution and functions of occludin and ezrin by type III toxins and ExoS, the consequences of type III toxins and ExoS on PMN migration through the epithelium, and the conditions in vivo that are conducive to their expression are established. Detailed experimental methods and statistical analyses are provided herein.

Calpains are recruited into lipid rafts. TLR2, asialoGM1, and c-Src are recruited into a caveolin-1 scaffolded lipid raft shortly following bacterial stimulation of airway cells (2). These lipid rafts are linked to the plasma membrane and attached to the cytoskeleton (75). This spatial co-localization of receptors, kinases and their association with membrane components facilitates the local generation of Ca²⁺ fluxes from the membrane bound stores as well as the efficient activation of downstream signals. As TLR2-associated Ca²⁺ flux activates calpain, calpain can be in close association with the TLR2-lipid raft and kinases. This can be tested by screening for the presence of calpain among the components of the TLR2 lipid raft following P. aeruginosa stimulation. 16HBE cell lysates can be harvested at timed intervals following stimulation, separated into triton soluble and insoluble fractions, and screened by immunoblot for caveolin-1, c-Src, TLR2 and calpain with an isotype control (2). Flotillin can be used as a marker for the raft fraction.

Calpains are Activated by TLR2 Signals

To demonstrate that calpain mobilization and activation is TLR2 dependent, several strategies to eliminate TLR2 signaling can be tested. 16HBE cells expressing the TLR2YY mutant that is incapable of signaling can be used along with a thapsigargin (+) control that signals in the absence of TLR2. A TLR2 truncation mutant lacking the cytoplasmic tail is also available for analysis. Primary airway epithelial cells cultivated from tlr2^{-/-} mice can be used in confirmatory studies. Lipid raft fractions of the airway cells can be screened for the mobilization of calpain; immunoblots can be done to identify calpain cleavage products, such as occludin, E-cadherin or ezrin.

Alternative Mechanisms of Calpain Activation

Other pathways involving the activation of PI3K and PLCγ have been shown to activate calpains. EGFR signaling stimulates MAPKs and in doing so induces calpain phosphorylation. TNFα signaling activates MAPKs (such as p38) that can alter the properties of epithelial junctions. This process can also involve calpain activity. However, as epithelial cells do not produce significant amounts of TNFα, the TNF cascade may not link bacterial signaling and modification in epithelial barrier function. Bacterial products do activate EGFR signaling. The role of EGFR in calpain activation can be addressed by stimulating epithelial cells with EGF and by testing calpain activation by bacteria in cells which do not express functional EGFR.

Calpain Activity Facilitates PMN Migration through Epithelial Junctions

Having demonstrated that calpain activity is stimulated by TLR2 signaling and that calpain inhibition limits the accumulation of PMNs in the murine lung, the manner by which calpain modified substrates in the epithelial junctions affect PMN trafficking can be examined. PMN migration across epithelial monolayers that lack calpain activity can be compared to migration across normal 16HBE cell controls. PMN migration across polarized 16HBE cells can be readily monitored by growing the cells on the basal surface of a Transwell, then flipping the insert so that PMN migration can be studied in the physiological direction from the baso-lateral to the apical surface in response to a chemoattractant such as fMLP or bacteria (FIG. 8). By inhibiting calpain expression or activity, PMN migration can be diminished.

siRNA to Calpain

Calpain expression can be blocked by introducing siRNAs for calpains 1 and 2 and compared with a scrambled siRNA control. Lack of calpain expression can be documented by immunoblot, and by growing the cells in a polarized fashion with an air-liquid interface on transwells. To accomplish this, cell lines that stably express calpain 1 and 2 siRNAs can be constructed. This can be done using a retroviral transfection system which has been previously used to construct cell lines that stably express TLR2 siRNA. Human PMNs can be applied to the wells (flipped upside down)—from the basolateral side, while a chemoattractant, such as heat killed PAO1 or fMLP (a standard PMN chemoattractant) is added to the apical chamber. PMN transmigration can be evaluated by monitoring myeloperoxidase in the apical chamber as well as by hematoxylin-eosin staining of the contents of the apical compartment and quantification of PMNs in a hemacytometer. Paracellular permeability can be assessed by monitoring the permeability of the monolayers to fluorescent dextrans 3,000 and 40,000 MW.

Biochemical Inhibitors of Calpain

Biochemical inhibitors of calpain can be tested to confirm the siRNA results. Wells can be treated with calpeptin (20 μM), a cell permeable calpain inhibitor, or with ALLN (20 μM), a calpain specific protease inhibitor and PMN migration can be quantified using the myeloperoxidase assay. As a positive control, cells can be treated with cytochalasin D (20 μM for 30 min. prior to stimulation), which depolymerizes actin filaments and serves to open the junctions.

Alternative Mechanisms

There are additional proteases in these airway cells involved in mobilization of phagocytes into the lung that can participate in the paracellular changes in permeability. Such proteases include ADAM 10, TACE (ADAM17) and MMPs. Monolayers with can be incubated the TACE inhibitor TAPI, with TACE siRNA, or with the general protease inhibitor GM6001 and compared with an untreated control to monitor PMN transmigration in response to PAO1 and P3C. These proteases can contribute to changes in paracellular permeability, but will not be specifically activated by TLR2 signals.

Effects of PMNs on Junctional Components

Migrating PMNs can alter the properties of the epithelial junctions by themselves. PMNs affect the distribution of endothelial components as they traverse endothelial junctions, although migration through MDCK monolayers demonstrated no differences in ZO-1 or F actin. To assess how PMN migration itself affects the airway epithelial junctions, the distribution of junctional proteins and permeability properties of monolayers can be compared in the presence of PMNs migrating in response to apical fMLP to PMNs incubated without a chemoattractant and compared with wells stimulated by the addition of PAO1 or P3C. The movement of PMNs through the junctions can initiate changes in junctional proteins. At selected intervals the effects of PMNs on epithelial permeability can be assessed by permeability studies by (1) testing TER and the accumulation of fluorescent dextran in the basal compartment, and (2) confocal imaging to follow the distribution of occludin, ZO-1, E-cadherin, ezrin and calpain in the airway cells. To determine if PMNs activate calpain, epithelial cells can be pretreated with the calpain inhibitor calpeptin or BAPTA/AM and permeability to dextran and PMNs can be quantified.

PMNs and Ca²⁺ Fluxes

If PMNs activate calpain, initiation of epithelial Ca²⁺ fluxes can assessed for PMN migration or apposition to epithelial cells. Screening studies can be performed by adding activated PMNs to Fluo-3/AM loaded 16HBE cells under a fluorescence microscope and following changes in epi-fluorescence over several minutes. To determine if PMN movement through the paracellular junctions elicits changes in [Ca⁻²]ᵢ, epithelial cells grown on inverted Transwells can be directly imaged. The cells can be loaded with Fluo3/AM and PMNs can be applied to basal surface and fMLP applied apically while on the microscope stage. The wells can then be directly imaged with digital images taken over 10 minutes and changes in [Ca²⁺]i can be monitored. Controls without fMLP and without PMNs can be performed.

Further analysis of the epithelial cells’ response to PMNs can be performed by confocal imaging to identify changes in occludin, actin, E-cadherin and ezrin to assess if PMN evoked changes in Ca²⁺ activate similar responses to those induced by TLR2.

Monolayers with diminished calpain expression will be less permeable to PMNs. The activation of this endogenous cascade will not impair the integrity of the epithelial barrier and the addition of an exogenous effector, such as a bacterial product, can significantly alter permeability. Because PMNs can transmigrate without substantially altering the state of the epithelium, reversible changes in the distribution of junctional proteins can examined.

Regulation of Calpain Activity

As a protease with many potential substrates, calpain must be highly regulated. As demonstrated in the activation of fibroblast motility, one mechanism of calpain regulation is by phosphorylation at ser50 by ERK. This phosphorylation only occurs when calpain is in proximity to membranes. As ERK is a component of the TLR2 cascade, ERK-mediated phosphorylation of calpain can be examined to determine if it is initiated as part of this pathway.

Co-Immunoprecipitation of p-ERK and Calpain

Co-immunoprecipitation can be done using a triton insoluble lipid raft fraction of 16HBE cells, before and after exposure to bacteria. Immunoblots for calpain, anti-phospho serine and p-ERK can be done. ERK can be transiently associated with calpain within the context of a lipid raft and that calpain is phosphorylated on serine 50.

Inhibition of ERK Blocks Calpain Activity

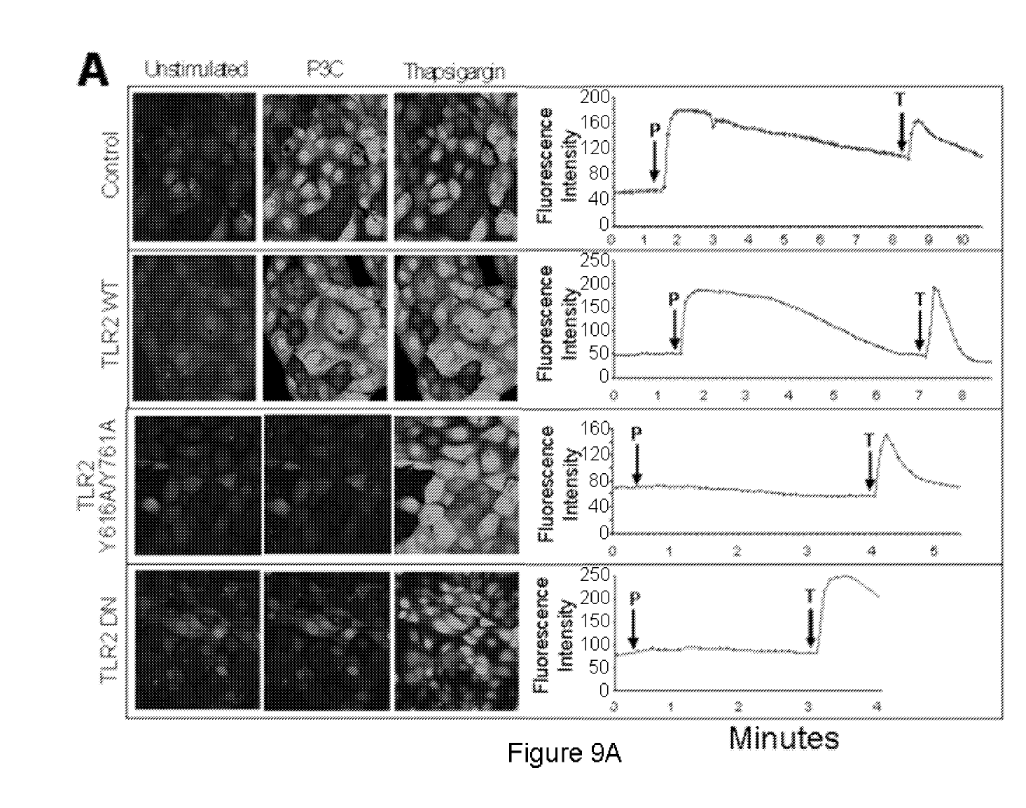

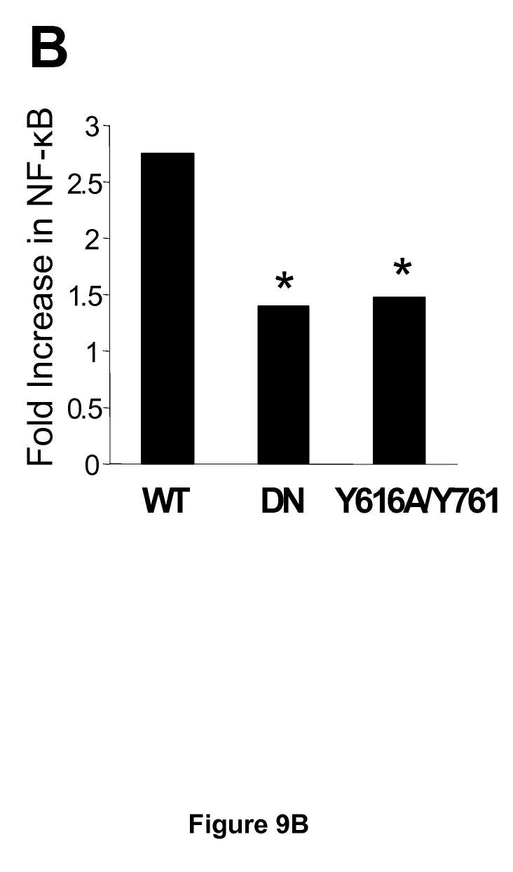

If calpain activation requires ERK, the ERK inhibitors U0126 and PD98059 can be used to block calpain activity. This can be measured using (1) the synthetic fluorescent calpain substrate shown in FIG. 9, (2) by monitoring activity in airway cells stimulated with thapsigargin or with P3C and treated with the ERK inhibitors or (3) with calpeptin, a calpain inhibitor control. The generation of the calpain cleavage products, such as the 45 kDa occludin fragment and the 100 kDa E-cadherin fragment, can be identified by immunoblotting. The results can be confirmed by testing the effects of expression of a DN MEK (the kinase upstream of ERK) construct as compared with a vector control (12), and monitoring calpain activity in the cells with a fluorescent substrate.

Site Directed Mutagenesis

The importance of phosphorylation of the predicted target of the ERK MAPK activity, at serine 50 on calpain can be established by constructing a mutant. Site directed mutagenesis can be performed to change the serine to an alanine The construct can then be used to test calpain activity of the mutant versus the wild type using the fluorescent substrate (shown in FIG. 9). This series of experiments will demonstrate that ERK MAP activity is important for activating calpain and that the calpain2 serine 50 is the target of ERK. The involvement of other MAPKs such as p38 can be studied in an analogous fashion.

Alternative Strategies

Calpastatin is the endogenous inhibitor of calpains, present in airway cells and changes in the calpastatin-calpain interaction can be important in regulating the activation of calpain in response to bacterial stimulation. The association of calpastatin and calpain in airway cells can be followed kinetically after bacterial stimulation to determine when the complex is disassociated and if this affects calpain phosphorylation. Antibodies to calpastatin are commercially available. Other analysis can involve the decrease calpastatin using siRNA techniques to determine effects on calpain activity and paracellular permeability.

Another approach can be used to activate calpain through a separate pathway and determine if it similarly targets the same epithelial substrates. EGF has been shown to mobilize calpain and also acts by stimulating ERK phosphorylation. These airway cells express EGFR the ability of EGF to activate calpain through ERK phosphorylation can be tested.

Identification of Calpain Substrates

A large number of cytoskeletal proteins and associated kinases have been identified as substrates for the calpains. While cleavage is specific, the sequences on either side of the m-calpain cleavage sites can vary. Calpain mediated cleavage of the β integrins occurs in regions flanking conserved NPXY/NXXY domains. Talin, an actin-integrin linker closely related to the ERM family, is also a calpain target with defined cleavage products. Based on the preliminary data identifying calpain targets in the epithelial junctions, calpain mediated cleavage of occludin and ezrin can be examined. E-cadherin, also a substrate for MMPs and TACE, is processed by calpain. However, much of its trafficking and endocytic recycling has been well characterized and the focus can be on junctional components that are less well understood.

Occludin

Occludin is present in the immunological synapse and participates in immune signaling. Occludin is involved in PMN transmigration across endothelial barriers although specific domains and mechanisms remain to be identified. It is regulated at the level of transcription, by phosphorylation, cleavage and endocytic recycling. The manner in which occludin and its processing by calpain participates in regulating tight junction function in response to bacterial stimuli can be determined.

Occludin Processing by Calpain

Occludin can be present in lipid raft associated with the tight junction and can targeted by activated calpain. Occludin can be cleaved by calpain, internalized and recycled through a caveolin-1 dependent endocytosis as occurs in MDCK cells when actin depolymerization is stimulated.

Occludin is Present in a Caveolin-1 Associated Lipid Raft with Calpain

Triton soluble and insoluble fractions of PAO1 stimulated 16HBE cells can be immunoblotted for occludin and calpain. Calpain will be mobilized into the triton insoluble fraction following bacterial stimulation, along with caveolin-1 and occludin. Co-immunoprecipitation experiments can be performed to determine if, in addition to the association of calpain, caveolin-1, TLR2 or additional kinases are present. This can be done in 16HBE and primary small airway epithelial cells.

Identification of Calpain Binding

The calpain binding site on occludin will be identified. The endogenous calpain inhibitor calpastatin is known to bind to the TIPPXYR (SEQ ID NO: 46) sequence. As a very similar sequence EYPPIT (SEQ ID NO: 47) is found in the C-terminus of occludin (421-427). The first four residues will be mutated to alanines and occludin-calpain binding can be monitored by immunoprecipitation and by the generation of the 45 kDa occludin fragment.

Identification of Calpain Cleavage Sites

The calpain cleavage site on occludin is intracellular, since calpain is intracellular. A 45 kDa occludin fragment is generated by calpain, limiting the number of potential cleavage sites. As occludin has been cloned and various domains readily expressed on plasmid constructs, a series of deletion mutants can be made to define which occludin domains contain the calpain cleavage site.

Endocytosis of Occludin is Stimulated by Bacterial Ligands

A variety of stimuli, including actin polymerization, activate caveolin-1 mediated endocytosis of occludin. The pathway involved has been well studied and occludin can be similarly recycled in airway cells. Following TLR2 activation, caveolin-1 mediated endocytosis of occludin will occur and be detected by association with the recycling endosomal marker Rab 11, or the late endosomal marker LAMP-1 and be documented by co-immunoprecipitation. Intracellular co-localization can be detected by confocal imaging using GFP-labeled Rabs, as has been demonstrated in MDCK cells. Each of these proteins is relatively abundant in airway cells, and confocal co-localization of these proteins has been successful in other cell types. Immunogold labeling, often used to track endocytic pathways, can also be used.

To document that this is an endocytic process, the effects of dynasore, a GTPase inhibitor that blocks endocytic recycling as well as a dynamin II DN K44A construct can be tested. This analysis will demonstrate that occludin is lost from the junctions and fails to associate with Rab 11 in the presence of 0.4M sucrose.

Calpain-Mediated Cleavage for Stimulation of Occludin Endocytosis

Calpain cleavage can initiate the endocytic processing of occludin. This can be established by (1) blocking the activity of calpain with calpeptin, (2) using 16HBE cells that express calpain 1 and 2 siRNA, and (3) adding the intracellular Ca²⁺ chelator BAPTA/AM which blocks calpain activity. Cells can be imaged by confocal microscopy and changes in the distribution of occludin and co-localization with Rabs 5 and 11 can be determined.

Occludin Phosphorylation as a Signal for Endocytosis

Many calpain targets are phosphorylated and the site of phosphorylation can limit calpain access to its substrate. Several kinases are involved in the phosphorylation of occludin. Phosphorylation is necessary for its trafficking to the membrane (Talavera, J Gen Virol 85, 1801 (Jul. 1, 2004, 2004)). Occludin has several potential sites for both tyrosine and ser/threonine phosphorylation. Tyrosine phosphorylation a consequence of PI3K activity has been associated with the dissociation of occludin from the tight junction. Loss of binding to ZO-1 (Tang et al., Infect. Immun. 64, 37 (Jan. 1, 1996, 1996)) also initiates endocytosis, although the consequences of phosphorylation appear to vary depending upon the cell type and the nature of the stimulus. LPS stimulates bile duct cells activating tyrosine phosphorylation of occludin and decreased TER, whereas Src inhibition and tyrosine kinase inhibitors cause an increase in permeability (Troisfontaines, G. R. Cornelis, Physiology 20, 326 (Oct. 1, 2005, 2005)). Thus, P-occludin is can be involved in regulation of airway cell permeability and may influence calpain cleavage. Occludin is a substrate for c-Src (Tunggal et al., EMBO J 24, 1146 (Mar. 23, 2005, 2005)), which is active in the TLR2 pathway and accumulates in TLR2 associated lipid rafts in response to bacterial ligands (Balachandran et al., J. Clin. Invest. 117, 419 (Feb. 1, 2007, 2007)). Thus, it can be determine if occludin is phosphorylated by c-Src.

c-Src Phosphorylation of Occludin in Response to TLR2 Signals

Co-immunoprecipitation studies can be done to see if c-Src family kinases associate with occludin in stimulated cells but not controls. Changes in electrophoretic mobility and by immunoblotting with anti-tyrosine phosphate specific antibody (4G10) can be used to examine any changes in tyrosine phosphorylation status. As occludin is hyperphosphorylated, the identification of specific sites of phosphorylation or relative levels of phosphorylation can be determined. The effects of PP1 and PP2 on thapsigargin treated cells, in which calpains are activated without involving TLR2, can be tested.

ERK Mediated Phosphorylation of Occludin

The C-terminal tail of occludin binds phospho-ERK (Turner, Am J Pathol 169, 1901 (Dec. 1, 2006, 2006)). To determine if ERK phosphorylates occludin in response to TLR2 signaling and thapsigargin, and whether it affects mobilization in response to P. aeruginosa, Occludin will be immunoprecipitated from the triton soluble and insoluble fractions of 16HBE cells stimulated with PAO1 or P3C in the presence of ERK inhibitor PD98059 or UO126. Immunoblots can be done with anti-serine/threonine-phosphate specific antibodies. The association of occludin with calpain (and cleavage) can be monitored by immunoprecipitation and immunoblotting.

Alternative Approaches

There are several other kinases that modify occludin and the focus of this work can be on the kinases already known to be activated in the TLR2 signaling pathway. PI3K is also a membrane associated kinase. It is a potential candidates that is found in airway cells and activated by TLRs. As described herein, other upstream signals can be needed for ERK activation and stimulation of the EGFR pathway can be tested as a different mechanism to activate ERK.

Occludin Effects on PMN Transmigration

The biologically important effect of occludin endocytosis from the tight junction can be an enhancement of PMN migration through the epithelial barrier. While several junctional proteins are apparently processed by calpain and contribute to changes in epithelial paracellular permeability, the contribution of calpain processing of occludin on epithelial barrier function can be tested. An occludin mutant can be constructed that lacks the site(s) necessary for calpain cleavage (as described herein). The mutant protein, cloned on a pcDNA vector can be overexpressed in 16HBE cells and polarized monolayers can be formed. PMN migration in response to fMLP, P3C and P. aeruginosa can be quantified and compared to controls expressing wild type occludin. If a specific calpain target is not readily identified, endocytosis can be alternatively blocked using cells stimulated in the presence of dynasore, or by incubating cells with a calpain inhibitor to determine effects on occludin and PMN migration. Controls can include the use of cell lines transfected with the pcDNA vector, the cells transfected with dynasore to block endocytosis, and with calpain inhibitors.

Occludin is involved in the regulation of epithelial permeability and this can be controlled at several levels. Because TLR2 initiated changes in occludin are being examined, this can allow a definition of the effects linked to this specific Ca²⁺ pathway. Multiple kinases target occludin and these experiments can demonstrate how the interaction of calpain and occludin facilitate PMN migration in response to bacterial pathogens.

Calpain Targets Ezrin

The second calpain target that will be studied in detail is ezrin. Ezrin is the Foxj1 dependent ERM that is targeted to the apical surface of airway epithelial cells (Izumi et al., J. Cell Biol. 166, 237 (Jul. 19, 2004, 2004)). In T cells the inactivation (de-phosphorylation) of ezrin causes a de-anchoring associated with decreased cell rigidity (Jia et al., Am J Physiol Lung Cell Mol Physiol 287, L428 (Aug. 1, 2004, 2004)). Ezrin is linked to signaling events that involve changes in the distensibility of the cytoskeleton (Ivetic, A. J. Ridley, Immunology 112, 165 (Jun. 1, 2004, 2004)). Bacterial ligands that signal through TLR2 to activate calpain can mediated cleavage of ezrin and terminate its actin-linker activity and contribute toward increased PMN migration across tight junctions.

Ezrin Dynamics and Lipid Rafts

Since calpain must be mobilized to the membrane to achieve a sufficiently high local concentration of Ca²⁺ for activation, ezrin must also be in the context of the same lipid raft (as is the case in B cells) (Jia et al., Am J Physiol Lung Cell Mol Physiol 287, L428 (Aug. 1, 2004, 2004)) for it to serve as a substrate. There are both cytoplasmic and membrane associated pools of ezrin. 16HBE cells can be fractionated at timed intervals following bacterial stimulation and triton X-100 soluble and insoluble fractions can be isolated. The fractions can be immunoblotted for ezrin, calpain, E-cadherin and ZO-1 as controls. A flotillin marker can be included to identify the lipid raft fraction. Bacterial stimulation can induce ezrin dissociation from the raft and increased accumulation in the cytoplasmic pool.

Ezrin is Cleaved by Calpain

To verify that calpain cleaves ezrin, the generation of the ezrin cleavage products in cells that lack calpain activity can be monitors. Calpain activity can be blocked by (1) treating cells with BAPTA/AM, a Ca²⁺ chelator; (2) treating cells with calpeptin, the cell permeable calpain inhibitor, and (3) expressing calpain 1 and 2 siRNA and as compared with a scrambled siRNA control. Thapsigargin treated cells can be used as a positive control for Ca²⁺ dependence. To confirm the importance of TLR2 activation the ezrin cleavage, products can be monitored in cells expressing the TLR2YY mutation. Ezrin cleavage can increase the distensibility of the paracellular junctions by decreasing the number of links between the junctional proteins and actin. To test this, it can be established that ezrin contributes to barrier properties of airway epithelial cells.

Effects of Ezrin Linker Activity—Analysis of a Constitutively Active Mutant

Ezrin phosphorylation maintains its plasma membrane-actin linker function by inhibiting the spontaneous interactions of the C and N-termini (Huber, M. S. Balda, K. Matter, J. Biol. Chem. 275, 5773 (Feb. 25, 2000, 2000)). Calpain-mediated cleavage of ezrin can inhibit this linker function. To document the importance of ezrin in dynamically regulating the plasma membrane-cytoskeletal association, an ezrin mutant construct that behaves as if it is constitutively phosphorylated (and hence is unable to dissociate from the cytoskeleton in response to stimuli) can be used. Threonine 567 phosphorylation of ezrin is associated with the open configuration. Threonine 567 can be substituted with aspartate to mimic phosphorylation and result in a constitutively active protein (Jia et al., Am J Physiol Lung Cell Mol Physiol 287, L428 (Aug. 1, 2004, 2004)). Site directed mutagenesis can be performed by substituting thr567 for aspartate and adding a GFP-label. This mutant-labeled ezrin, cloned on pcDNA, can be transfected into 16HBE cells using the AMAXA Nucleofector and compared to cells transfected with a pcDNA construct with the wild type ezrin linked to GFP. The mutant can be verified for an inability to be further threonine phosphorylated by performing immunoprecipitation and immunoblotting with anti-phosphothreonine antibodies.

Trafficking Studies

The distribution of the wild type and constitutively active ezrin can be followed by confocal imaging at intervals following bacterial stimulation. ZO-1, E-cadherin and actin patterns of distribution can be examined as well. Ezrin has several well defined interactions with actin (Rao et al. Biochem J 368, 471 (Dec. 1, 2002, 2002)). The C-terminus of ezrin has sites which regulate PI3K, by binding the p85 component and RhoGTPases, by interacting with the Rho GDP dissociation inhibitor (J. Clin. Invest. 113, 1482 (May 15, 2004, 2004)) both of which affect actin polymerization. To determine if constitutive activation of ezrin alters its interactions with actin, monolayers exposed to the wild type or constitutively active ezrin can be treated with phalloidin to visualize effects on actin polymerization/depolymerization. Constitutive activation can be dominant and the internalization of ezrin can be impaired in cells with constitutively active ezrin.

Effects of Constitutively Active Ezrin on Permeability

Ezrin internalization is associated with increased paracellular permeability. The permeability properties of the cells expressing wild type or the constitutively active ezrin can be compared by monitoring (1) TER, (2) dextran permeability, and (3) PMN transmigration across the barrier. These assays can demonstrate that phosphorylated ezrin, as mimicked by the mutant ezrin described herein contributes to barrier function. Cells transfected with the wild type pcDNA construct can be used as a control.

Alternative Methods—Inhibition of Ezrin Phosphorylation

An alternative approach can be to use site directed mutagenesis to change the threonine 567 to an alanine and prevent phosphorylation. This can result in an inactive form of ezrin which can fail to respond to bacterial signaling. Cells expressing this mutant ezrin can be less of a barrier to PMN transmigration.

These experiments can determine how localization and phosphorylation of occludin and ezrin contribute to the maintenance of barrier function following bacterial stimulation. Characterizing the dynamics of their physiological responses to conserved bacterial stimuli can help explain how PMNs gain access to the airway lumen, even in response to non-invasive opportunists.

### REFERENCES

Adamo, R., Sokol, S., Soong, G., Gomez, M. I. & Prince, A. Pseudomonas aeruginosa flagella activate airway epithelial cells through asialoGM1 and toll-like receptor 2 as well as toll-like receptor 5. Am J Respir Cell Mol Biol 30, 627-634 (2004).
Bojarski, C., et al. The specific fates of tight junction proteins in apoptotic epithelial cells. J Cell Sci 117, 2097-2107 (2004).
Boyum, A. Isolation of mononuclear cells and granulocytes from human blood. Isolation of monuclear cells by one centrifugation, and of granulocytes by combining centrifugation and sedimentation at 1 g. Scand J Clin Lab Invest Suppl 97, 77-89 (1968).
Bryant, D. M. & Stow, J. L. The ins and outs of E-cadherin trafficking Trends Cell Biol 14, 427-434 (2004).
Bryant, D. M., et al. EGF induces macropinocytosis and SNX1-modulated recycling of E-cadherin. J Cell Sci 120, 1818-1828 (2007).
Chun, J. & Prince, A. Activation of Ca2+-dependent signaling by TLR2. J Immunol 177, 1330-1337 (2006).
Cuzzocrea, S., et al. Calpain inhibitor I reduces the development of acute and chronic inflammation. Am J Pathol 157, 2065-2079 (2000).
D'Souza-Schorey, C. Disassembling adherens junctions: breaking up is hard to do. Trends in Cell Biology 15, 19-26 (2005).
Dwyer-Nield, L. D., Miller, A. C., Neighbors, B. W., Dinsdale, D. & Malkinson, A. M. Cytoskeletal architecture in mouse lung epithelial cells is regulated by protein-kinase C-alpha and calpain II. Am J Physiol 270, L526-534 (1996).
Feldman, G. J., Mullin, J. M. & Ryan, M. P. Occludin: structure, function and regulation. Adv Drug Deliv Rev 57, 883-917 (2005).
Fettucciari, K., et al. Group B Streptococcus induces macrophage apoptosis by calpain activation. J Immunol 176, 7542-7556 (2006).

Fournier, B. & Philpott, D. J. Recognition of Staphylococcus aureus by the innate immune system. Clin Microbiol Rev 18, 521-540 (2005).
Franco, S. J. & Huttenlocher, A. Regulating cell migration: calpains make the cut. J Cell Sci 118, 3829-3838 (2005).
Glading, A., Lauffenburger, D. A. & Wells, A. Cutting to the chase: calpain proteases in cell motility. Trends Cell Biol 12, 46-54 (2002).
Goll, D. E., Thompson, V. F., Li, H., Wei, W. & Cong, J. The calpain system. Physiol Rev 83, 731-801 (2003).
Gomez, M. I., et al. Staphylococcus aureus protein A induces airway epithelial inflammatory responses by activating TNFR1. Nat Med 10, 842-848 (2004).
Harhaj, N. S. & Antonetti, D. A. Regulation of tight junctions and loss of barrier function in pathophysiology. Int J Biochem Cell Biol 36, 1206-1237 (2004).
Hopkins, A. M., Walsh, S. V., Verkade, P., Boquet, P. & Nusrat, A. Constitutive activation of Rho proteins by CNF-1 influences tight junction structure and epithelial barrier function. J Cell Sci 116, 725-742 (2003).
Hordijk, P. Endothelial signaling in leukocyte transmigration. Cell Biochem Biophys 38, 305-322 (2003).
Huber, D., Balda, M. S. & Matter, K. Occludin modulates transepithelial migration of neutrophils. J Biol Chem 275, 5773-5778 (2000).
Ichiyasu, H., et al. Matrix metalloproteinase-9-deficient dendritic cells have impaired migration through tracheal epithelial tight junctions. Am J Respir Cell Mol Biol 30, 761-770 (2004).

Kuchay, S. M. & Chishti, A. H. Calpain-mediated regulation of platelet signaling pathways. Curr Opin Hematol 14, 249-254 (2007).

Li, Q., Park, P. W., Wilson, C. L. & Parks, W. C. Matrilysin shedding of syndecan-1 regulates chemokine mobilization and transepithelial efflux of neutrophils in acute lung injury. Cell 111, 635-646 (2002).
Lopez-Santiago, L. F., et al. Sodium channel beta2 subunits regulate tetrodotoxin-sensitive sodium channels in small dorsal root ganglion neurons and modulate the response to pain. J Neurosci 26, 7984-7994 (2006).
McGuire, J. K., Li, Q. & Parks, W. C. Matrilysin (matrix metalloproteinase-7) mediates E-cadherin ectodomain shedding in injured lung epithelium. Am J Pathol 162, 1831-1843 (2003).
Mege, R. M., Gavard, J. & Lambert, M. Regulation of cell-cell junctions by the cytoskeleton. Curr Opin Cell Biol 18, 541-548 (2006).
Mehta, D. & Malik, A. B. Signaling mechanisms regulating endothelial permeability. Physiol Rev 86, 279-367 (2006).
Muller, S. L., et al. The tight junction protein occludin and the adherens junction protein alpha-catenin share a common interaction mechanism with ZO-1. J Biol Chem 280, 3747-3756 (2005).
Nuzzi, P. A., Senetar, M. A. & Huttenlocher, A. Asymmetric localization of calpain 2 during neutrophil chemotaxis. Molecular Biology of the Cell 18, 795-805 (2007).
Orrington-Myers, J., et al. Regulation of lung neutrophil recruitment by VE-cadherin. Am J Physiol Lung Cell Mol Physiol 291, L764-771 (2006).
Parks, W. C., Wilson, C. L. & Lopez-Boado, Y. S. Matrix metalloproteinases as modulators of inflammation and innate immunity. Nat Rev Immunol 4, 617-629 (2004).
Rajan, S., et al. Pseudomonas aeruginosa induction of apoptosis in respiratory epithelial cells: analysis of the effects of cystic fibrosis transmembrane conductance regulator dysfunction and bacterial virulence factors. Am J Respir Cell Mol Biol 23, 304-312 (2000).
Ratner, A. J., et al. Cystic fibrosis pathogens activate Ca2+-dependent mitogen-activated protein kinase signaling pathways in airway epithelial cells. J Biol Chem 276, 19267-19275 (2001).
Reutershan, J., Basit, A., Galkina, E. V. & Ley, K. Sequential recruitment of neutrophils into lung and bronchoalveolar lavage fluid in LPS-induced acute lung injury. Am J Physiol Lung Cell Mol Physiol 289, L807-815 (2005).
Rios-Doria, J. & Day, M. L. Truncated E-cadherin potentiates cell death in prostate epithelial cells. Prostate 63, 259-268 (2005).
Rios-Doria, J., et al. The role of calpain in the proteolytic cleavage of E-cadherin in prostate and mammary epithelial cells. J Biol Chem 278, 1372-1379 (2003).
Saitou, M., et al. Complex phenotype of mice lacking occludin, a component of tight junction strands. Molecular Biology of the Cell 11, 4131-4142 (2000).
Schulzke, J. D., et al. Epithelial transport and barrier function in occludin-deficient mice. Biochim Biophys Acta 1669, 34-42 (2005).
Shao, H., et al. Spatial localization of m-calpain to the plasma membrane by phosphoinositide biphosphate binding during epidermal growth factor receptor-mediated activation. Mol Cell Biol 26, 5481-5496 (2006).
Shen, L. & Turner, J. R. Actin depolymerization disrupts tight junctions via caveolae-mediated endocytosis. Molecular Biology of the Cell 16, 3919-3936 (2005).
Skerrett, S. J., Liggitt, H. D., Hajjar, A. M. & Wilson, C. B. Cutting edge: myeloid differentiation factor 88 is essential for pulmonary host defense against Pseudomonas aeruginosa but not Staphylococcus aureus. J Immunol 172, 3377-3381 (2004).
Soong, G., Reddy, B., Sokol, S., Adamo, R. & Prince, A. TLR2 is mobilized into an apical lipid raft receptor complex to signal infection in airway epithelial cells. J Clin Invest 113, 1482-1489 (2004).
Tang, H. B., et al. Contribution of specific Pseudomonas aeruginosa virulence factors to pathogenesis of pneumonia in a neonatal mouse model of infection. Infect Immun 64, 37-43 (1996).
Wachtel, M., et al. Occludin proteolysis and increased permeability in endothelial cells through tyrosine phosphatase inhibition. J Cell Sci 112 (Pt 23), 4347-4356 (1999).
Wagner, J. G. & Roth, R. A. Neutrophil migration mechanisms, with an emphasis on the pulmonary vasculature. Pharmacol Rev 52, 349-374 (2000).
Wan, H., et al. Tight junction properties of the immortalized human bronchial epithelial cell lines Calu-3 and 16HBE14o. Eur Respir J 15, 1058-1068 (2000).
Wu, Z., Nybom, P. & Magnusson, K. E. Distinct effects of Vibrio cholerae haemagglutinin/protease on the structure and localization of the tight junction-associated proteins occludin and ZO-1. Cell Microbiol 2, 11-17 (2000).
Yu, A. S., et al. Knockdown of occludin expression leads to diverse phenotypic alterations in epithelial cells. Am J Physiol Cell Physiol 288, C1231-1241 (2005).
Zhu, L., Li, X., Zeng, R. & Gorodeski, G. I. Changes in tight junctional resistance of the cervical epithelium are associated with modulation of content and phosphorylation of occludin 65-kilodalton and 50-kilodalton forms. Endocrinology 147, 977-989 (2006).
1. R. Adamo, S. Sokol, G. Soong, M. I. Gomez, A. Prince, Am. J. Respir. Cell Mol. Biol. 30, 627 (May 1, 2004, 2004).
2. P. Balachandran et al., J. Clin. Invest. 117, 419 (Feb. 1, 2007, 2007).
3. S. Basuroy, A. Seth, B. Elias, A. P. Naren, R. Rao, Biochem J 393, 69 (Jan. 1, 2006, 2006).
4. S. Basuroy et al., J. Biol. Chem. 278, 11916 (Mar. 28, 2003, 2003).
5. C. Beisswenger, C. B. Coyne, M. Shchepetov, J. N. Weiser, J. Biol. Chem. 282, 28700 (Sep. 28, 2007, 2007).
6. D. S. Black, J. B. Bliska, Mol Microbiol 37, 515 (Aug. 1, 2000, 2000).
7. D. M. Bryant, J. L. Stow, Trends Cell Biol 14, 427 (Aug. 1, 2004, 2004).
8. D. O. Y. Cheung, K. Halsey, D. P. Speert, Infect. Immun. 68, 4585 (Aug. 1, 2000, 2000).
9. J. Chun, A. Prince, J Immunol 177, 1330 (Jul. 15, 2006, 2006).
10. J. C. Comolli, L. L. Waite, K. E. Mostov, J. N. Engel, Infect. Immun. 67, 3207 (Jul. 1, 1999, 1999).
11. C. B. Coyne, L. Shen, J. R. Turner, J. M. Bergelson, Cell Host & Microbe 2, 181 (2007).
12. R. D'Angelo et al., Mol. Biol. Cell, E06 (Sep. 19, 2007, 2007).
13. S. Dewitt, M. Hallett, J Leukoc Biol 81, 1160 (May 1, 2007, 2007).
14. S. Epelman et al., J. Immunol. 173, 2031 (Aug. 1, 2004, 2004).
15. L. Falzano et al., Mol Microbiol 9, 1247 (Sep. 1, 1993, 1993).
16. S. Faure et al., Nat Immunol 5, 272 (Mar. 1, 2004, 2004).
17. G. J. Feldman, J. M. Mullin, M. P. Ryan, Adv Drug Deliv Rev 57, 883 (Apr. 25, 2005, 2005).
18. H. Feltman et al., Microbiology 147, 2659 (Oct. 1, 2001, 2001).
19. B. Fievet, D. Louvard, M. Arpin, Biochim Biophys Acta 1773, 653 (May 1, 2007, 2007).
20. S. J. Franco, A. Huttenlocher, J. Cell Sci. 118, 3829 (Sep. 1, 2005, 2005).
21. S. J. Franco et al., Nat Cell Biol 6, 977 (Oct. 1, 2004, 2004).
22. D. W. Frank, Mol Microbiol 26, 621 (November, 1997).
23. A. Glading et al., Mol. Cell. Biol. 24, 2499 (Mar. 15, 2004, 2004).
24. A. Glading, D. A. Lauffenburger, A. Wells, Trends Cell Biol 12, 46 (Jan. 1, 2002, 2002).
25. D. E. Goll, V. F. Thompson, H. Li, W. E. I. Wei, J. Cong, Physiol. Rev. 83, 731 (Jul. 1, 2003, 2003).
26. M. I. Gomez et al., Nat Med 10, 842 (Aug. 1, 2004, 2004).
27. M. I. Gomez, M. O. Seaghdha, A. S. Prince, EMBO J 26, 701 (Feb. 7, 2007, 2007).
28. M. I. Gomez et al., J Immunol 175, 1930 (Aug. 1, 2005, 2005).
29. A. L. Goodman et al., Dev Cell 7, 745 (Nov. 1, 2004, 2004).
30. S. Gopalakrishnan, N. Raman, S. J. Atkinson, J. A. Marrs, Am J Physiol Cell Physiol 275, C798 (Sep. 1, 1998, 1998).
31. N. Gupta et al., Nat Immunol 7, 625 (Jun. 1, 2006, 2006).
32. J. A. Guttman, F. N. Samji, Y. Li, A. W. Vogl, B. B. Finlay, Infect. Immun. 74, 6075 (Nov. 1, 2006, 2006).
33. A. R. Hauser et al., Crit Care Med 30, 521 (March, 2002).
34. A. M. Hopkins, S. V. Walsh, P. Verkade, P. Boquet, A. Nusrat, J. Cell Sci. 116, 725 (Feb. 15, 2003, 2003).
35. A. M. Hopkins, S. V. Walsh, P. Verkade, P. Boquet, A. Nusrat, J. Cell Sci. 116, 725 (Feb. 15, 2003, 2003).
36. W. Hua, D. Sheff, D. Toomre, I. Mellman, J. Cell Biol. 172, 1035 (Mar. 27, 2006, 2006).
37. T. Huang et al., J Cell Sci 116, 4935 (Dec. 15, 2003, 2003).
38. D. Huber, M. S. Balda, K. Matter, J. Biol. Chem. 275, 5773 (Feb. 25, 2000, 2000).
39. A. Ivetic, A. J. Ridley, Immunology 112, 165 (Jun. 1, 2004, 2004).
40. G. Izumi et al., J. Cell Biol. 166, 237 (Jul. 19, 2004, 2004).
41. P. A. Janmey, Physiol Rev 78, 763 (Jul. 1, 1998, 1998).
42. H. P. Jia et al., Am J Physiol Lung Cell Mol Physiol 287, L428 (Aug. 1, 2004, 2004).
43. M. R. Jones, B. T. Simms, M. M. Lupa, M. S. Kogan, J. P. Mizgerd, J Immunol 175, 7530 (Dec. 1, 2005, 2005).
44. T. Joseph, D. Look, T. Ferkol, Am J Physiol Lung Cell Mol Physiol 288, L471 (Mar. 1, 2005, 2005).
45. G. Kale, A. P. Naren, P. Sheth, R. K. Rao, Biochem Biophys Res Commun 302, 324 (Mar. 7, 2003, 2003).
46. M. Koltzscher, C. Neumann, S. Konig, V. Gerke, Mol. Biol. Cell 14, 2372 (Jun. 1, 2003, 2003).
47. M. Koss et al., J. Immunol. 176, 1218 (Jan. 15, 2006, 2006).
48. R. Krall, J. Sun, K. J. Pederson, J. T. Barbieri, Infect. Immun. 70, 360 (Jan. 1, 2002, 2002).
49. M. C. Lebart, Y. Benyamin, FEBS J 273, 3415 (Aug. 1, 2006, 2006).
50. V. T. Lee, R. S. Smith, B. Tummler, S. Lory, Infect. Immun. 73, 1695 (Mar. 1, 2005, 2005).
51. B. F. Lillemeier, J. R. Pfeiffer, Z. Surviladze, B. S. Wilson, M. M. Davis, PNAS 103, 18992 (Dec. 12, 2006, 2006).
52. S. Louvet-Vallee, Biol Cell 92, 305 (Aug. 1, 2000, 2000).
53. T. E. Machen, Am J Physiol Cell Physiol 291, C218 (Aug. 1, 2006, 2006).
54. M. Mall, B. R. Grubb, J. R. Harkema, W. K. O'Neal, R. C. Boucher, Nat Med 10, 487 (May 1, 2004, 2004).
55. A. W. Maresso, M. R. Baldwin, J. T. Barbieri, J. Biol. Chem. 279, 38402 (Sep. 10, 2004, 2004).
56. A. W. Maresso, Q. Deng, M. S. Pereckas, B. T. Wakim, J. T. Barbieri, Cell Microbiol 9, 97 (Jan. 1, 2007, 2007).
57. T. Maretzky et al., PNAS 102, 9182 (Jun. 28, 2005, 2005).
58. K. S. Matlin, Am J Physiol Cell Physiol 288, C1191 (Jun. 1, 2005, 2005).
59. C. G. Mayhall, Clin Infect Dis 45, 712 (Sep. 15, 2007, 2007).
60. J. K. McGuire, Q. Li, W. C. Parks, Am J Pathol 162, 1831 (Jun. 1, 2003, 2003).
61. J. K. McGuire, Q. Li, W. C. Parks, Am. J. Pathol. 162, 1831 (Jun. 1, 2003, 2003).
62. D. Mehta, A. B. Malik, Physiol. Rev. 86, 279 (Jan. 1, 2006, 2006).
63. E. Melloni et al., J. Biol. Chem. 281, 24945 (Aug. 25, 2006, 2006).
64. A. Muir et al., Am. J. Respir. Cell Mol. Biol. 30, 777 (Jun. 1, 2004, 2004).
65. S. L. Muller et al., J. Biol. Chem. 280, 3747 (Feb. 4, 2005, 2005).
66. A. J. Newton, T. Kirchhausen, V. N. Murthy, PNAS 103, 17955 (Nov. 21, 2006, 2006).
67. D. A. Potter et al., J. Cell Biol. 141, 647 (May 4, 1998, 1998).
68. A. S. Prince, J. P. Mizgerd, J. Wiener-Kronish, J. Bhattacharya, Am J Physiol Lung Cell Mol Physiol 291, L297 (Sep. 1, 2006, 2006).
69. S. D. P. Rabin, J. L. Veesenmeyer, K. T. Bieging, A. R. Hauser, Infect. Immun. 74, 2552 (May 1, 2006, 2006).
70. R. K. Rao, S. Basuroy, V. U. Rao, K. J. Karnaky Jr, A. Gupta, Biochem J 368, 471 (Dec. 1, 2002, 2002).
71. A. J. Ratner et al., J. Biol. Chem. 276, 19267 (May 25, 2001, 2001).
72. C. M. P. Ribeiro et al., J. Biol. Chem. 280, 17798 (May 6, 2005, 2005).
73. C. L. Rocha, J. Coburn, E. A. Rucks, J. C. Olson, Infect. Immun. 71, 5296 (Sep. 1, 2003, 2003).
74. C. L. Rocha, E. A. Rucks, D. M. Vincent, J. C. Olson, Infect. Immun. 73, 5458 (Sep. 1, 2005, 2005).
75. R. T. Sadikot, T. S. Blackwell, J. W. Christman, A. S. Prince, Am. J. Respir. Crit. Care Med. 171, 1209 (Jun. 1, 2005, 2005).
76. L. Saiman, A. Prince, J Clin Invest 92, 1875 (Oct. 1, 1993, 1993).
77. M. Saitou et al., Mol. Biol. Cell 11, 4131 (Dec. 1, 2000, 2000).
78. G. Schlunck et al., Mol. Biol. Cell 15, 256 (Jan. 1, 2004, 2004).
79. E. E. Schneeberger, R. D. Lynch, Am J Physiol Cell Physiol 286, C1213 (Jun. 1, 2004, 2004).
80. H. Shao et al., Mol. Cell. Biol. 26, 5481 (Jul. 15, 2006, 2006).
81. C. M. Shaver, A. R. Hauser, Infect. Immun. 72, 6969 (Dec. 1, 2004, 2004).
82. A. Shcherbina, A. Bretscher, D. M. Kenney, E. Remold-O'Donnell, FEBS Lett 443, 31 (Jan. 22, 1999, 1999).
83. L. Shen, J. R. Turner, Mol. Biol. Cell 16, 3919 (Sep. 1, 2005, 2005).
84. P. Sheth, N. Delos Santos, A. Seth, N. F. LaRusso, R. K. Rao, Am J Physiol Gastrointest Liver Physiol 293, G308 (Jul. 1, 2007, 2007).
85. S. J. Skerrett, C. B. Wilson, H. D. Liggitt, A. M. Hajjar, Am J Physiol Lung Cell Mol Physiol 292, L312 (Jan. 1, 2007, 2007).
86. G. Soong, B. Reddy, S. Sokol, R. Adamo, A. Prince, J. Clin. Invest. 113, 1482 (May 15, 2004, 2004).
87. S. Sousa et al., Cell Microbiol 9, 2629 (Nov. 1, 2007, 2007).
88. U. Steinhusen et al., J. Biol. Chem. 276, 4972 (Feb. 9, 2001, 2001).
89. V. S. Subramanian, J. S. Marchant, D. Ye, T. Y. Ma, H. M. Said, Am J Physiol Cell Physiol, 00309.2007 (Sep. 13, 2007, 2007).
90. J. Sun, J. T. Barbieri, J. Biol. Chem. 279, 42936 (Oct. 8, 2004, 2004).
91. D. Talavera, A. M. Castillo, M. C. Dominguez, A. E. Gutierrez, I. Meza, J Gen Virol 85, 1801 (Jul. 1, 2004, 2004).
92. H. B. Tang et al., Infect. Immun. 64, 37 (Jan. 1, 1996, 1996).
93. P. Troisfontaines, G. R. Cornelis, Physiology 20, 326 (Oct. 1, 2005, 2005).
94. J. A. Tunggal et al., EMBO J 24, 1146 (Mar. 23, 2005, 2005).
95. J. R. Turner, Am J Pathol 169, 1901 (Dec. 1, 2006, 2006).
96. M. L. Urbanowski, E. D. Brutinel, T. L. Yahr, Infect. Immun. 75, 4432 (Sep. 1, 2007, 2007).
97. R. E. Vance, A. Rietsch, J. J. Mekalanos, Infect. Immun. 73, 1706 (Mar. 1, 2005, 2005).
98. M. Wachtel et al., J. Cell Sci. 112, 4347 (Dec. 1, 1999, 1999).
99. H. Wan et al., J. Clin. Invest. 104, 123 (Jul. 1, 1999, 1999).
100. H. Wang et al., Biochem Biophys Res Commun 333, 496 (Jul. 29, 2005, 2005).
101. P. D. Ward, R. R. Klein, M. D. Troutman, S. Desai, D. R. Thakker, J. Biol. Chem. 277, 35760 (Sep. 13, 2002, 2002).
102. V. Wong, Am J Physiol Cell Physiol 273, C1859 (Dec. 1, 1997, 1997).
103. B. Yan, D. A. Calderwood, B. Yaspan, M. H. Ginsberg, J. Biol. Chem. 276, 28164 (Jul. 20, 2001, 2001).
104. A. S. Yap, W. M. Brieher, B. M. Gumbiner, Annu Rev Cell Dev Biol 13, 119 (Jan. 1, 1997, 1997).
105. Y. Zhang, Q. Deng, J. T. Barbieri, J. Biol. Chem. 282, 13022 (Apr. 27, 2007, 2007).
106. Z. Zheng et al., J. Biol. Chem. 282, 6136 (Mar. 2, 2007, 2007).
107. L. Zhu, X. Li, R. Zeng, G. I. Gorodeski, Endocrinology 147, 977 (Feb. 1, 2006, 2006).

What is claimed is:

1. A method for reducing inflammation in a subject, the method comprising administering at least one calpain inhibitor to the respiratory epithelial cells in the airway of the subject, wherein the at least one calpain inhibitor is administered in an amount sufficient to inhibit leukocyte transmigration across epithelial tight junctions between the respiratory epithelial cells, and wherein the calpain inhibitor(s) is capable of inhibiting the protease activity of calpain 1 and/or calpain 2.

2. The method of claim 1, wherein the at least one calpain inhibitor reduces the activity of both calpain 1 and calpain 2.

3. The method of claim 1, wherein the at least one calpain inhibitors are capable of inhibiting calpain 1 and/or calpain 2 such that calpain-mediated cleavage of both occludin and E-cadherin is inhibited.

4. The method of claim 1, wherein the at least one calpain inhibitors are capable of inhibiting calpain 1 and/or calpain 2 such that calpain-mediated cleavage of occludin, E-cadherin and Ezrin is inhibited.

5. The method of claim 1, wherein the subject suffers from: asthma or an asthma exacerbation; chronic obstructive pulmonary disease; an opportunistic pathogenic infection of cystic fibrosis; a respiratory infection; pneumonia; a ventilator-associated pneumonia, an obstructive airway disease or condition; an eosinophil related disorder; bronchial condition; or pulmonary inflammation.

6. The method of claim 1, wherein the at least one calpain inhibitor inhibits E-cadherin cleavage, occludin cleavage, or ezrin cleavage, or any combination thereof

7. The method of claim 1, wherein the at least one calpain inhibitor comprises a small molecule, a protein, a peptide, a peptidomimetic, small interfering RNA, a short hairpin RNA, a microRNA, and an anti-calpain antibody, and derivative thereof

8. The method of claim 7, wherein the nucleic acid sequence of the small interfering RNA is selected from the sequences shown in any of SEQ ID NOs: 27-42.

9. The method of claim 7, wherein the nucleic acid sequence of the small interfering RNA has at least 95%, 96%, 97%, 98%, or 99% nucleic acid sequence identity the sequences shown in any of SEQ ID NOs: 27-42.

10. The method of claim 1, wherein the calpain inhibitor acts on or binds to an active site of calpain.

11. The method of claim 10, wherein the calpain inhibitor is selected from the group comprising: Calpain Inhibitor Peptide, Calpain Inhibitor I, N-acetly-L-L-norleucinal, ALLN, Calpain Inhibitor II, N-acetly-L-L-methional, ALLM, Calpain Inhibitor III, Calpain Inhibitor IV, Calpain Inhibitor V, Calpeptin, benzyloxycarbonyldipeptidyl aldehyde, trans-Epoxy succinyl-L-leucylamido-(4-guanidino) butane, and Z-Leu-Leu-CHO, and derivatives thereof.

12. The method of claim 10, wherein the calpain inhibitor is selected from the group comprising: E64D, SJA6017, N-(4-fluorophenylsulfonyl)-L-valyl-L-leucinal, AK295, benzyloxycarbony-Leu-aminobutyric acid-CONH(CH₂)₃-morpholine, AK275, and benzyloxycarbony-Leu-Abu-CONH-CH2CH3, and derivatives thereof.

13. The method of claim 1, wherein the calpain inhibitor comprises calpastatin or a calpastatin peptide mimetic.

14. The method of claim 1, wherein the calpain inhibitor binds to a calcium binding domain of calpain.

15. The method of claim 14, wherein the calpain inhibitor is selected from the group comprising: PD 150606, [3-(4-Iodophenyl)-2-mercapto-(benzyloxycarbonyl)-2-propenoic acid], PD 1151746, 3-(5-fluoro-3-indolyl)-2-mercapto-(benzyloxycarbonyl)-2-propenoic acid, or any derivatives thereof.

16. The method of claim 1, wherein the calpain inhibitor is a polypeptide comprising the amino acid sequence shown in SEQ ID NO: 44 or 45.

17. The method of claim 1, wherein the calpain inhibitor is a polypeptide comprising an amino acid sequence having at least 70% sequence identity to the amino acid sequence shown in SEQ ID NO: 44 or 45.

18. The method of claim 1, wherein the calpain inhibitor is administered with one or more additional therapeutic agent(s), wherein the additional therapeutic agent(s) can be administered at the same or at a different time than the calpain inhibitor.

19. The method of claim 18, wherein the additional therapeutic agent comprises an anti-bacterial substance, an anti-viral substance, an anti-inflammatory substance, a bronchodilatory substance, an antihistamine substance or an anti-tussive substance.

20. The method of claim 19, wherein the anti-viral substance comprises ritonavir, saquinavir, indinavir, nelfinavir, amprenavir, or any derivative thereof

21. The method of claim 18, wherein the additional therapeutic agent comprises a steroid, a beta-2 agonists, a PDE4 inhibitor, a LTD4 antagonist, an anticholinergic agent, a corticosteroid, a steroid anti-inflammatory agent or a bronchodilator.

22. The method of claim 1, wherein the additional therapeutic agent comprises a chemokine receptor antagonist.

23. The method of claim 1, wherein the leukocyte is a white blood cell, a neutrophil, a lymphocyte, a monocyte, a basophile, a macrophage, a dendritic cell, a mast cell, a phagocyte or an eosinophil, or any combination thereof.

24. The method of claim 1, wherein the calpain inhibitor is administered orally, parenterally, by inhalation, intranasally, topically, subcutaneously, intramuscularly, rectally or by intrapulmonary injection.

25. The method of claim 1, wherein the calpain inhibitor is linked to a targeting moiety that specifically binds to the surface of a cell in the pulmonary epithelium, a cell in the lower respiratory airway or a cell in the upper respiratory airway of the subject.

26. The method of claim 1, wherein the method further comprises administering a calcium chelator to the subject.

27. The method of claim 1, wherein the method further comprises administering a calcium blocker to the subject.
